SEQUENCE LISTING (59 sequences)

SEQ ID NO: 1 - DNA, 18 bp, Artificial Sequence
  Motif repeat
  misc_feature (1)..(1): n= G or T
  misc_feature (7)..(7): n= T or A
  misc_feature (8)..(8): n= T or A
  misc_feature (9)..(10): n is a, c, g, or t
  misc_feature (11)..(11): n= G or T
  misc_feature (17)..(17): n= T or A
  misc_feature (18)..(18): n= T or A
nttacannnnnttacann

SEQ ID NO: 2 - protein, 126 aa, Artificial Sequence
  N-RpaB
MENRKEKILVVDDEASIRRILETRLAMIGYEVVTAADGEEALITFRNATPDLVVLDVMMPKLDGYGVCQELRKESDVPIIMLTALGDVADRITGLELGADDYVVKPFSPKELEARIRSVLRRVEKS

SEQ ID NO: 3 - DNA, 327 bp, Cyanobacterium ABICyano1
tgagaaaaagtgtaaacaaatattaagaaaaagatcagaaaaatttaacaacacgtaataaaaaaatgcgtcactacgggttataaatttacatgaaaggttaaaacacttttctgagacgattttgataaaaaagttgtcaaaaaattaagtttctttacaaatgcttaacaaaaacttggttttaagcacaaaataagagagactaatttgcagaagttttacaaggaaatcttgaagaaaaagatctaagtaaaacgactctgtttaaccaaaatttaacaaatttaacaaaacaaactaaatctattaggagattaactaagc

SEQ ID NO: 4 - DNA, 409 bp, Cyanobacterium ABICyano1
cttgaaaaagttgaggtattaatagagcttgataaatgataataaaaacagatttagctcttattttaagggaaaaagaaataaataaaatattagtaaatatcaaaaatatcagcctttcaaaaataatttgactcttttcaaaaaaaaatgttatctttaaggtatgctttaaaccttaaatacttctattggtaacactgttctcaatcttatttcagattttcccattgagcataaataaaatattaagcagaagtagaaaaggttgatattagcaataataaaaattaacaataaaatgtgaaaacagattactactgattatttattgccatgagctaattagtaataatttgtcttttttgatcgaaaaatgaaattttttaagcggaggaactgaaaatta

SEQ ID NO: 5 - DNA, 205 bp, Cyanobacterium ABICyano1
tagagtatgataaaatgacaaggaaaggattattttctcttgtttaaattctcaagattcttatgcttatttattttatgtaagtgtctcttttccttgaaatagaaagaaaaaagtggctaattttgagaaaagctaacaacgctttggttaactaaaaatcaaaagtgagattactgatcgcttaagaaatggagtattgatt

SEQ ID NO: 6 - DNA, 338 bp, Cyanobacterium ABICyano1
gcagttagataaataagtaatgagcgggagaaataggggcaaatggccattcgcccctacagggaggtggcaggtgttagggtgtttaggggatgaggtgatgagggtagagggagataaggtgtcgggtttcagatttcaggttttagaagaaagtaacgagtaattatcaactattcactattcactattgcctgttgcccttctctccttgaaatataaaaaaatgtaaaaatatcattaagaaaagtaacaaaataaacagaaaggttgacaaagttgacgctttaatatccgtatgttagctttataacaacgaaatcaacggaggagtgaaa

SEQ ID NO: 7 - DNA, 411 bp, Cyanobacterium ABICyano1
tatttatatataaactcgaataaaattatcaatataaagtcaaactatatctatcctattttaactgctattggtaagtcccttaattagtgttggggtgaatagattttaaaagggcaaacccccctttatcctccctcgagaggggggagggcaaaaggcaaggggcaagggaaaaattaagaattaagaattaaaaactccgaacacctgtaggggcgaatagccattcgcttcccctcatccccccatctccccaacaccctaagcccctactcgttactcatttatttacatcatttatttacatcattaagaaaagtaacaaattttgacaagtagtcttttgacaggaaaaagcaaattctcgaagatgaaaacaatagaaaaaaattcaatcttacagtaacg

SEQ ID NO: 8 - DNA, 214 bp, Cyanobacterium ABICyano1
agagttatatttacatagtgtgtgcgagtaagggcaacttttgtaggtagatgaataaacctcaaattactcatcttaaaagacgatatttttaatctattcttctgtaataaaatacttctttcgatagagatatttaatacttttgagagatgaaaataatttcaataattgtcatgatagagagtaagtgcaaataagaaaaaattgattt

SEQ ID NO: 9 - DNA, 211 bp, Cyanobacterium ABICyano1
gtgatatttggtttattctatattttccttaagtaaaaattcagtcatgagggaaacttttgttaaaatttgctttaaattaataggaagatcattaagaaaatcttaaaaagattgagtttttagatcgaaattattgaagaaaaattaacaggggttctgctcaaaattttattaaattactctactgtagtaaaggagaaattttatt

SEQ ID NO: 10 - DNA, 380 bp, Cyanobacterium ABICyano1
ataaccaatgggacttgaattttagatccatttatttaattctatttttgttacatttctttatattaatcagaattatgttactttgttttgttttatgtcgttaccttattgaagaaagagtggatgagaaggtaaatgacggggcataaatatcgattcgttgtcagaataagctgttttattcacttaactggttgtttgccaatttctccctaattcccataacttgtataactaaatttaataatcaattttagtaaattaagaataggttaaaagtagtatttagaattaagttaactttaataaatttcctgtatttttttatagaaaaaagtataaaataaaaacatatcaaaaaagtttgaaatgacaat

SEQ ID NO: 11 - DNA, 542 bp, Cyanobacterium ABICyano1
gaatagttgataattactcgttactcattactcacttaaacctgccacctgatacctgccacctctccccccatcacctcatcccctcaacattccgaaccccttgacactttgaactaaaattgtattaaagtgcaaatctggacggggttaaccagtgtgacttataatagtaaacgctgttttttataataaataagctaaatatttaaaaactatgagtaaatatacactaaatggtactagacgtaagcagaaaagaacctccggtttccgcgcccgtatgagaaccaaaaatggtagaaaagtaattcaagctcgtcgtaataagggtagaaaaagattagcagtataaaattactgttaaataaggaagctaagtttagcattttaagtttgatattactaatcattaaatttactgtgaaatataggtgggactaccatcaaagcatcgactgaaacggcgtttaaatttccaatctgtttatcaacagggtattcgccgctctagtcgttattttattgtccgagggttacgg

SEQ ID NO: 12 - DNA, 281 bp, Artificial Sequence
  Generalized PnirA sequence
  misc_feature (1)..(116): n is a, c, g, or t
  misc_feature (132)..(138): n is a, c, g, or t
  misc_feature (154)..(168): n is a, c, g, or t
  misc_feature (183)..(204): n is a, c, g, or t
  misc_feature (211)..(265): n is a, c, g, or t
  misc_feature (277)..(278): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcaaaaaacgaatnnnnnnnatgtgtaaaaagaaannnnnnnnnnnnnnngtagtcaaagttacnnnnnnnnnnnnnnnnnnnnnntaatgtnnnnnnnnnnnnnnnnnnnnnnnnnnnnnnnnnnnnnnnnnnnnnnnnnnnnnnnccgaggacaaannatg

SEQ ID NO: 13 - DNA, 282 bp, Artificial Sequence
  modified PnirA
  misc_feature (1)..(116): n is a, c, g, or t
  misc_feature (132)..(138): n is a, c, g, or t
  misc_feature (154)..(168): n is a, c, g, or t
  misc_feature (183)..(204): n is a, c, g, or t
  misc_feature (211)..(265): n is a, c, g, or t
  misc_feature (278)..(279): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcaaaaaacgaatnnnnnnnatgtgtaaaaagaaannnnnnnnnnnnnnngtagtcaaagttacnnnnnnnnnnnnnnnnnnnnnntaatgtnnnnnnnnnnnnnnnnnnnnnnnnnnnnnnnnnnnnnnnnnnnnnnnnnnnnnnnggaggatcagccnnatg

SEQ ID NO: 14 - DNA, 281 bp, Artificial Sequence
  modified PnirA
  misc_feature (1)..(116): n is a, c, g, or t
  misc_feature (132)..(138): n is a, c, g, or t
  misc_feature (154)..(168): n is a, c, g, or t
  misc_feature (183)..(204): n is a, c, g, or t
  misc_feature (211)..(265): n is a, c, g, or t
  misc_feature (277)..(278): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcaaaaaacgcatnnnnnnnatgcgtaaaaagcatnnnnnnnnnnnnnnngtaatcaaagttacnnnnnnnnnnnnnnnnnnnnnntaatatnnnnnnnnnnnnnnnnnnnnnnnnnnnnnnnnnnnnnnnnnnnnnnnnnnnnnnnccgaggacaaannatg

SEQ ID NO: 15 - DNA, 282 bp, Artificial Sequence
  modified PnirA
  misc_feature (1)..(116): n is a, c, g, or t
  misc_feature (132)..(138): n is a, c, g, or t
  misc_feature (154)..(168): n is a, c, g, or t
  misc_feature (183)..(204): n is a, c, g, or t
  misc_feature (211)..(265): n is a, c, g, or t
  misc_feature (278)..(279): n is a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgcaaaaaacgcatnnnnnnnatgcgtaaaaagcatnnnnnnnnnnnnnnngtaatcaaagttacnnnnnnnnnnnnnnnnnnnnnntaatatnnnnnnnnnnnnnnnnnnnnnnnnnnnnnnnnnnnnnnnnnnnnnnnnnnnnnnnggaggatcagccnnatg

SEQ ID NO: 16 - DNA, 87 bp, Artificial Sequence
  inducible PcorT
  misc_feature (4)..(10): n is a, c, g, or t
  misc_feature (57)..(71): n is a, c, g, or t
catnnnnnnngtttactcaaaaccttgacattgacactaatgttaaggtttaggctnnnnnnnnnnnnnnncaagttaaaaagcatg

SEQ ID NO: 17 - DNA, 87 bp, Artificial Sequence
  modified PcorT
  misc_feature (4)..(10): n is a, c, g, or t
  misc_feature (57)..(71): n is a, c, g, or t
catnnnnnnngtttactcaaaaccttgacattgacactaatgttaaggtttaggctnnnnnnnnnnnnnnngaggataaaaagcatg

SEQ ID NO: 18 - DNA, 85 bp, Artificial Sequence
  inducible PcorT
  misc_feature (4)..(10): n is a, c, g, or t
  misc_feature (55)..(69): n is a, c, g, or t
catnnnnnnngtttactcaaaaccttgacattgactaatgttaaggtttagaatnnnnnnnnnnnnnnncaagttaaaaagcatg

SEQ ID NO: 19 - DNA, 87 bp, Artificial Sequence
  inducible PcorT
  misc_feature (4)..(10): n is a, c, g, or t
  misc_feature (57)..(71): n is a, c, g, or t
catnnnnnnngtttactcaaaaccttgacattgacactaatgttaaggtttagaatnnnnnnnnnnnnnnngaggataaaaaccatg

SEQ ID NO: 20 - DNA, 96 bp, Artificial Sequence
  inducible PsmtA
  misc_feature (1)..(8): n is a, c, g, or t
  misc_feature (35)..(38): n is a, c, g, or t
  misc_feature (69)..(80): n is a, c, g, or t
  misc_feature (87)..(93): n is a, c, g, or t
nnnnnnnnaatacctgaataattgttcatgtgttnnnntaaaaatgtgaacaatcgttcaactatttannnnnnnnnnnnggaggtnnnnnnnatg

SEQ ID NO: 21 - DNA, 96 bp, Artificial Sequence
  modified PsmtA
  misc_feature (1)..(8): n is a, c, g, or t
  misc_feature (35)..(38): n is a, c, g, or t
  misc_feature (69)..(78): n is a, c, g, or t
  misc_feature (90)..(93): n is a, c, g, or t
nnnnnnnnaatacctgaataattgttcatgtgttnnnntaaaaatgtgaacaatcgttcaactatttannnnnnnnnnaaggaggtgatnnnnatg

SEQ ID NO: 22 - DNA, 110 bp, Artificial Sequence
  modified PsmtA
  misc_feature (1)..(22): n is a, c, g, or t
  misc_feature (49)..(52): n is a, c, g, or t
  misc_feature (83)..(92): n is a, c, g, or t
  misc_feature (103)..(107): n is a, c, g, or t
nnnnnnnnnnnnnnnnnnnnnnaatacctgaataattgttcatgtgttnnnntaaaaatgtgaacaatcgttcaactatttannnnnnnnnnaaggaggtatnnnnnatg

SEQ ID NO: 23 - DNA, 381 bp, Synechococcus elongatus
atggaaaatcgcaaggaaaaaatcctcgttgtcgacgatgaagcgagcatccggcggattcttgaaactcggttggcgatgattggttacgaagttgtcaccgcagccgacggcgaagaagccctcatcaccttccgcaatgctacgccggatctcgtggtgctcgatgtgatgatgcccaagctcgatggctatggcgtttgccaagagctgcgcaaagagtcggacgttccgatcatcatgctgacagccttgggcgatgtggccgatcgcattacggggcttgagttgggagctgatgactacgtcgtcaaacccttctcgcctaaggaactagaagcgcgaatccgctcggtgctgcgtcgggtcgaaaaaagctag

SEQ ID NO: 24 - protein, 244 aa, Synechococcus elongatus
MENRKEKILVVDDEASIRRILETRLAMIGYEVVTAADGEEALITFRNATPDLVVLDVMMPKLDGYGVCQELRKESDVPIIMLTALGDVADRITGLELGADDYVVKPFSPKELEARIRSVLRRVEKSGANGIPSSGVIQINSIRIDTNKRQVYKGDERIRLTGMEFSLLELLVSRSGEPFSRAEILQEVWGYTPERHVDTRVVDVHISRLRAKLEDDPGNPELILTARGTGYLFQRIVEPGEEGR

SEQ ID NO: 25 - DNA, 735 bp, Synechococcus elongatus
ttggaaaatcgcaaggaaaaaatcctcgttgtcgacgatgaagcgagcatccggcggattcttgaaactcggttggcgatgattggttacgaagttgtcaccgcagccgacggcgaagaagccctcatcaccttccgcaatgctacgccggatctcgtggtgctcgatgtgatgatgcccaagctcgatggctatggcgtttgccaagagctgcgcaaagagtcggacgttccgatcatcatgctgacagccttgggcgatgtggccgatcgcattacggggcttgagttgggagctgatgactacgtcgtcaaacccttctcgcctaaggaactagaagcgcgaatccgctcggtgctgcgtcgggtcgaaaaaagcggtgctaatggcatccccagttcgggcgtcatccagatcaacagcatccgcatcgacaccaataagcgccaagtctacaaaggcgatgagcgcatccgtctgacgggcatggagttcagtttgttggaactgctggtcagccgctccggtgaaccttttagccgcgccgaaatcctgcaagaggtctggggctatacccccgagcgccacgtcgatacccgcgtagtcgatgtccacatctcgcggctgcgcgccaaattggaagacgatccgggcaaccctgagctcattctgacggcccgaggaaccggctacctcttccaacgcatcgttgaaccgggcgaagaagggcgttag

SEQ ID NO: 26 - protein, 248 aa, Synechococcus sp. WH 8102
MTATTPSKETILVVDDEASIRRILETRLSMIGYNVVTACDGTEALELFENTAPDLVVLDVMMPKLDGYGVCQELRKESDVPIVMLTALGDVADRITGLELGADDYVVKPFSPKELEARIRCVLRRVEKESVAGIPNSGVIQVSDLRIDTNKRQVFRADERIRLTGMEFSLLELLVSRSGEPFNRGEILKEVWGYTPERHVDTRVVDVHISRLRSKLEDDPANPELILTARGTGYLFQRIIDSVASEGP

SEQ ID NO: 27 - protein, 233 aa, Tolypothrix sp. PCC 7601
MVDDEASIRRILETRLSMIGYDVVTAGDGEEALETFRKADPDLVVLDVMMPKLDGYGVCQELRKESDVPIIMLTALGDVADRITGLELGADDYVVKPFSPKELEARIRSVLRRVDKTSASGIPSSGVIHVANIKIDTNKRQVYKGDERIRLTGMEFSLLELLVSRSGEAFSRSEILQEVWGYTPERHVDTRVVDVHISRLRAKLEDDPSNPELILTARGTGYLFQRIIEPGEE

SEQ ID NO: 28 - protein, 247 aa, Thermosynechococcus sp. NK55a
METHKEKILVVDDEASIRRILETRLSMIGYTVVTAADGEEALTTFRQEQPDLVVLDVMMPKLDGYGVCQELRKESDVPIIMLTALGDVADRITGLELGADDYVVKPFSPKELEARIRSVLRRIEKTNTSGIPSSGVIQVGNIRIDTNKRQVYKGDERIRLTGMEFMLLELLVGRSGEPFSRAEILEQVWGYTPERHVDTRVVDVHISRLRAKLEEDPSNPELILTARGTGYLFQRITEPGEASNKNQ

SEQ ID NO: 29 - protein, 243 aa, Arthrospira platensis
MENHKERILVVDDEASIRRILETRLSMIGYDVVTAADGEEALETFRLTEPDLVVLDVMMPKLDGYGVCQELRKESDIPIIMLTALGDVADRITGLELGADDYVVKPFSPKELEARIRSVLRRIDKNGASGIPSSGVIQIASIRIDTNKRQVYKGDERIRLTGMEFSLLELLVSRSGEPFSRSEILQEVWGYTPERHVDTRVVDVHISRLRAKLEDDPSNPELILTARGTGYLFQRIIDPSEVG

SEQ ID NO: 30 - DNA, 729 bp, Artificial Sequence
  Artificial DNA encoding Arthrospira RpaB
atggagaaccacaaggagcgcatcctggtggtggatgatgaggccagcatccgccgcatcctggagacccgcctgagcatgatcggctacgatgtggtgaccgccgccgatggcgaggaggccctggagaccttccgcctgaccgagcccgatctggtggtgctggatgtgatgatgcccaagctggatggctacggcgtgtgccaggagctgcgcaaggagagcgatatccccatcatcatgctgaccgccctgggcgatgtggccgatcgcatcaccggcctggagctgggcgccgatgattacgtggtgaagcccttcagccccaaggagctggaggcccgcatccgcagcgtgctgcgccgcatcgataagaacggcgccagcggcatccccagcagcggcgtgatccagatcgccagcatccgcatcgataccaacaagcgccaggtgtacaagggcgatgagcgcatccgcctgaccggcatggagttcagcctgctggagctgctggtgagccgcagcggcgagcccttcagccgcagcgagatcctgcaggaggtgtggggctacacccccgagcgccacgtggatacccgcgtggtggatgtgcacatcagccgcctgcgcgccaagctggaggatgatcccagcaaccccgagctgatcctgaccgcccgcggcaccggctacctgttccagcgcatcatcgatcccagcgaggtgggc

SEQ ID NO: 31 - protein, 244 aa, Cyanobacterium aponinum
METQKEKILVVDDEASIRRILETRLSMIGYDVVTAADGEDAIATFHETQPDLVVLDVMMPKLDGYGVCQELRKESDIPIIMLTALGDVADRITGLELGADDYVVKPFSPKELEARIRSVLRRVDKTGVAGIPSSGVISINSIRIDTNKRQVYKGDQRIRLTGMEFSLLELLVSKSGEPFSRSEILQEVWGYTPERHVDTRVVDVHVSRLRAKLEDDPSNPELILTARGTGYLFQRILEPGEKKK

SEQ ID NO: 32 - DNA, 732 bp, Artificial Sequence
  Artificial DNA encoding Cyanobacterium RpaB
atggagacccagaaggagaagatcctggtggtggatgatgaggccagcatccgccgcatcctggagacccgcctgagcatgatcggctacgatgtggtgaccgccgccgatggcgaggatgccatcgccaccttccacgagacccagcccgatctggtggtgctggatgtgatgatgcccaagctggatggctacggcgtgtgccaggagctgcgcaaggagagcgatatccccatcatcatgctgaccgccctgggcgatgtggccgatcgcatcaccggcctggagctgggcgccgatgattacgtggtgaagcccttcagccccaaggagctggaggcccgcatccgcagcgtgctgcgccgcgtggataagaccggcgtggccggcatccccagcagcggcgtgatcagcatcaacagcatccgcatcgataccaacaagcgccaggtgtacaagggcgatcagcgcatccgcctgaccggcatggagttcagcctgctggagctgctggtgagcaagagcggcgagcccttcagccgcagcgagatcctgcaggaggtgtggggctacacccccgagcgccacgtggatacccgcgtggtggatgtgcacgtgagccgcctgcgcgccaagctggaggatgatcccagcaaccccgagctgatcctgaccgcccgcggcaccggctacctgttccagcgcatcctggagcccggcgagaagaagaag

SEQ ID NO: 33 - DNA, 1413 bp, Synechococcus elongatus
gtgcgcgagatctgggttcagagcagctatctcgctgcaactatctttttctttctcggtttgaagcaactgggccatcctgccaccgctcgccgaggcaactatttgggtgctgcgggcatgggtttggcggtgctgggcaccgctttccatcctgacctccaagatccgtggttgccccttctcgccgtggggctgggcagcattgccggcattgtgtccgcccagagagtggtgatgacggccatgccccagatggtggccctcctcaatgggttggggggcgcagcttccgcactcgtggcgatcgcggagtacttgcgactctcccaggtgcccttggcagcaacacaacagctgccgaccctaattgcactgctgttgggcctgttgattggcggcgtcaccctcagtggcagtcttgttgctttcgccaaactccagggcttgatcagcggtcaacccctgaccttgcccagtcatcgactctggagcttgctcttgctgctgggttttgtcagcggtgcagtcctcttcttggtcgcgcctcaagccccgaccgctttggcggtgctgactgctctagcgatcgcaacactattgctggggctcttcttcgtcgcgccggtcgggggtggcgatatgccagtggtgattgcactgttgaactctctgtcaggtttggccgccagtgcagcaggctttgcagttaacaacaacctcctgatcattgctggcgcgctggtcggcgcctcaggactgatcctgacggtgatcatgtgcaaagccatgaaccgatcactaaccaatgtcctgttttcaggtttggttgctgctggtggtcaggctgatgggggaacgtcagcagcgggcgatcgcaatgctcgccagatcgattctgatgagggagcaatgctgctgggctacgcccgatcggtcgtgattgtaccgggctacggcatggcggttgctcaggcccaacatacagtgcgtgagttggcggagcatctccaacggctaggcgttgaggtgaaatatgccattcaccccgtagcgggtcggatgcccggtcacatgaatgtgttgttggcggaagccaatgtcccctacggtcagctctacgacctcgagtcgatcaactccgagtttgaacatacagatgttgcactggtgattggcgccaatgatgtcgtcaatcctgccgctcgcctcgataacagcagcccgatttacggtatgccgattttggatgtcgatcgtgctcgccaaacgattgtaattaagcgcagtatggcgaatggctatgccggcatcgataatccactcttctacggcgatcgcacggtgatgctgtttggcagtgctcgggaagtcatttcccaactcgtcggagctgtcaaagagttgtcttag

SEQ ID NO: 34 - protein, 470 aa, Synechococcus elongatus
MREIWVQSSYLAATIFFFLGLKQLGHPATARRGNYLGAAGMGLAVLGTAFHPDLQDPWLPLLAVGLGSIAGIVSAQRVVMTAMPQMVALLNGLGGAASALVAIAEYLRLSQVPLAATQQLPTLIALLLGLLIGGVTLSGSLVAFAKLQGLISGQPLTLPSHRLWSLLLLLGFVSGAVLFLVAPQAPTALAVLTALAIATLLLGLFFVAPVGGGDMPVVIALLNSLSGLAASAAGFAVNNNLLIIAGALVGASGLILTVIMCKAMNRSLTNVLFSGLVAAGGQADGGTSAAGDRNARQIDSDEGAMLLGYARSVVIVPGYGMAVAQAQHTVRELAEHLQRLGVEVKYAIHPVAGRMPGHMNVLLAEANVPYGQLYDLESINSEFEHTDVALVIGANDVVNPAARLDNSSPIYGMPILDVDRARQTIVIKRSMANGYAGIDNPLFYGDRTVMLFGSAREVISQLVGAVKELS

SEQ ID NO: 35 - DNA, 294 bp, Synechococcus elongatus
atgagcatcaccaattggttcgctgcactctttgtcttcaccttggcgagttttgcaggcttagaagtcattaataaagtgccgccaacgctacataccccgctgatgtcgggggcgaatgcgatttctggcattgtcctgctgggtgccctgacactcttgggtggtgaggggccaccgctcctgaaaggactcgcgtttacggctgccattctggccacggtgaatgtggtgggtggcttttgggtaaccgatcgcatgttggcgatgttcaaagcacgggagacgaagtga

SEQ ID NO: 36 - protein, 97 aa, Synechococcus elongatus
MSITNWFAALFVFTLASFAGLEVINKVPPTLHTPLMSGANAISGIVLLGALTLLGGEGPPLLKGLAFTAAILATVNVVGGFWVTDRMLAMFKARETK

SEQ ID NO: 37 - DNA, 1227 bp, Synechococcus elongatus
atgcgatcaccggtcaactgcccagcagaaggctgtttcgacaacgcggcggggtcccgtcagcaaaccgtaatgaagattttgattccctgcgagccagggctgcaagagcgccgtgtcgctatgctgccctcccagcttgcacgcttaaaacaactaactggcgggacgatctccgttcaaagtggcgccggccagccgttaggactcagcgatcgcgactatcaagaggcgggagcggaagtcagtgatcaggaagagcaactgtggcgacaagctgatctggtgctctcgataggcccttggcgcagcgaacaagccacgcccctcaattggctgcgatcgggagcttgcctgattggtttgctcgatccttggtcccagacagacagcctcgatcgcctcgcggcgcagggtgtctcggcgcttagcttggaactgattcctcgcattagtcgtgcccaagcgatggatgtcttatcctctcaggccagcattattggctatcgcagcgtcctgctgggagccacggccctagggcgctatctaccgatgctgactacggctgcgggtacaattccgcccgccaaggtgctggtcttgggagctggagtagctggactgcaggcgatcgccacggcccgtcgccttggggcggtcgtcagcgcctttgatgtacgcaagaccgctggcgaagaggttcaaagcttaggtgccaagttcatcgatgtggacctgagcgagtctgctgaggctagcgggggctacgctaaacagctcaacgaagacagcatggcgcgcttgcgggcggggcttttgccccatctccaacaggccgatttgattgtgacgacggcacaagttccgggtaagccggctccttgcctcatctccgccgagatggtggcccagctacagccaggcacggtcattgtcgatgctgccgcagaacggggtggcaactgcgaactgacctgcgcgggccgcgaagttgatatcgatggcgtcaaggtactgggtcccactgcgatcgctgctgggctcccccacgatgccagcttcctgttcgggcgcaatttgctggctctgctaccgctcctctgccacgaaggtgagctgcggttgcccttggacgatgaaatcatcgccggctgctgtgttgtgaccgccggtgagcgtcgcttcccgccccaacctaaacccctgcctgtgccggaggctgtatga

SEQ ID NO: 38 - protein, 408 aa, Synechococcus elongatus
MRSPVNCPAEGCFDNAAGSRQQTVMKILIPCEPGLQERRVAMLPSQLARLKQLTGGTISVQSGAGQPLGLSDRDYQEAGAEVSDQEEQLWRQADLVLSIGPWRSEQATPLNWLRSGACLIGLLDPWSQTDSLDRLAAQGVSALSLELIPRISRAQAMDVLSSQASIIGYRSVLLGATALGRYLPMLTTAAGTIPPAKVLVLGAGVAGLQAIATARRLGAVVSAFDVRKTAGEEVQSLGAKFIDVDLSESAEASGGYAKQLNEDSMARLRAGLLPHLQQADLIVTTAQVPGKPAPCLISAEMVAQLQPGTVIVDAAAERGGNCELTCAGREVDIDGVKVLGPTAIAAGLPHDASFLFGRNLLALLPLLCHEGELRLPLDDEIIAGCCVVTAGERRFPPQPKPLPVPEAV

SEQ ID NO: 39 - protein, 582 aa, Synechococcus sp. WH 8103
MVVAAAAPKLSLQCEAIAGDTCTIRSLDWDRSRFDIEFGLRNGTTYNAFLVRGERTALIDTSHAKFRDTWIPLLKEQIEPTAIDVLIVSHTEPDHSGLIGDLIDLNPDIEIVGSKVALQFLQDQVHRPFKSRAVKSGEELDLGTNPESGIQHRFEFLSAPNLHWPDTIFSFDHGTGILYTCDAFGLHYCSDDVFDSDPGAIAPDFRFYYDCLMGPNARSVLQALKRMGGLPEINTIAVGHGPLLRHHLSHWVNDYREWSGQRSKGESYAAVCYLSQYGFSDRISQAIAHGIGKAEAQVQLVDLRATDPQELTALIGDAKAVVVPTWPAEPDGELQASIGTLLAALHPKQLVGVFDAFGGNDEPIDAVADQLRSQGQKQAFAPLRIRQLPQGSDYQRCEESGTDLGQLLTKEKAIAAMKSLDGDLDKALGRISGGLYVVTASQGEGESQRRSAMVASWVSQASFSPPGLTVAVAKDRAIEALMQVGDRFVLNVLREDNHQPLLRHFLKRFPPGADRFAGVNVLEGVADGGPVLTDALAYLSCRVEQRMEGPDHWIIYAVVEQGNVADAEASTAVHHRKVGNHY

SEQ ID NO: 40 - protein, 570 aa, Thermosynechococcus sp. NK55a
MPVTTTKRPPRLTLQVLDIAPETTAIRCLDWDRDRFDIEFALENGTTYNSFLIKGERIALVDTSHAKFGDRYLDQLWQLVNPSDLNYLIVSHTEPDHSGLVKDVLTKAPHVTVVASKVALQFLGDLIHQPFSQQQVKNGDRLDLGKGHVLEFVMAPNLHWPDTILTYDHGTQTLFTCDVFGAHFCNDDPFDSEPELLAPDFKFYYDCLMGPNARSVLSAFKRLEPLPPVQLVATGHGPLLRHHLDQWLESYRNWSQEQAKAATTVAIFYAANYGYSNALAEAIERGAAKTGVVVEKMDLLTAEPQDIRELTEIAAGIIIGTPPATAVAQTALSTIRAAAHAKQAIGVFESVADAEPAYPLLNQFRDAGLVPSFPVIQVTATPTDALFQQAEEAGTDMGQWLLRDRTVKQMKALNTDLDKALGRLSGGLYIITAQKGAINSAMLASWVAQASTEPLGVSIAVAKDRAIESFLHVGDTFVLNVLEEGNYQPLMRHFLKRFPPGADRFAHVKTYPASNGSPILADALAYLECTVVSRLDAHDHWIVYSTVDSGRVSKPDGMTAVHHRKVGNHY

SEQ ID NO: 41 - protein, 578 aa, Thermosynechococcus sp. NK55a
MLTETRPRDVQVAEIAPGILVLRSRTWDRLKFEVEYGRQQGTTSNSYLIQAPQPTLLDPPGESFTQIYLQELQQHIDLSQLRYLILSHVNSNRLATVKVLLEKAPQITIVCSKAAAVALRSAVGEQLNLWIPRAETPLELGGDRQLMFIAAATPRWPDGLMTVDPQNQIVFSDKLFGAHVCGDSLYDEQWKKLDEDRAYYFECLHAAQTRQVESILDRLAELTPPPRLYAPAHGPIVKFSRSRLFQDYRDWCQAQTAQDTKVALFYASAYGNTAILANAIAQGLTAAGVQVEAVNCETTPPAEMQALIHSSDGFIIGSPTLGGHMPTQVQTALGFILAEGSQTKLAGVFGSYGWSGEAIDDIEQKLVDAGYTLGFETLRVKFTPTASDLEKCQIAASEFAQALKKLRKSRTVRPPSLAEAQVDRTEQAVNRVVGSLCVLTTLPEGCFHLTQAAAILVSSVSQASFHPPGITVSLPQEWAESLCLVGDRFVLNILKEGSPLVRQFQQAQRLGEQQLATLGLKSAESGAPILLDALAYLECTVESRMNCGNHWLIYAVVESGELLQTSGLTAIQHRKTSS

SEQ ID NO: 42 - protein, 173 aa, Synechocystis sp. PCC 6803
MTVATDRQTVPPSAAHPSGDKRFKVLDATMKRNQFNQDALIEILHKAQEIFGYLEEDVLLYVARGLKLPLSRVFGVATFYHLFSLKPSGKHTCVVCLGTACYVKGAGDLLKTLDQEVHLKPGETTEDGQMSLVTARCIGACGIAPAVVYDGKVLGKQNDEAVLAAIQPWLSNS

SEQ ID NO: 43 - protein, 184 aa, Synechococcus elongatus
MDKIKLATVWLAGCSGCHMSFLDLDEWLIDLADGVEVVFSPVACDRKDYPEGVDLCLIEGAVANLDNLELLHQVRDRTRILVSFGDCAIHANVPGMRNLWGAESAAAVLERGYLELADTTPQLPNEPGIVPPLLNRVTPIHELVAIEHYLPGCPPPADRIRSLLQALLDQQTPPHSGRDLLKFG

SEQ ID NO: 44 - protein, 474 aa, Cyanothece sp. ATCC 51142
MSKTIVIDPVTRIEGHAKISIFLDDNGQVDDARFHVVEYRGFEKFCEGRPMWEMAGITARICGICPVSHLLASAKTGDKILAVKIPQPAEKLRRLMNLAQITQSHALSFFHLSSPDFLLGWDSDPAKRNIFGLIASDPELAKSGIRLRQFGQTIIELLGAKKIHAAWSVPGGVRSPLSKDSQQWIKERLPEAYETANNALSLFKKLLDRFETEVKTFGDFPSLFMGLVGEDGLWEHYGGHLRFSDSQGNIVADHLKEEDYQDFIGESVEKWSYLKFPYYKPLGYPQGIYRVGPLARLNICSHFGTEKADQELEEFRNRAGGVATSSFFYHYARLVEIIGCLERIERLMNDPDLLSTNTRAKAGINNLEGIGVSEAPRGTLFHHYNVDENGLIKKVNLIIATGNNNLAMNKTVTQIAKHYVKGNAMTEGMLNRVEAGIRCYDPCLSCSTHAAGQMPLHIQLFGVNGDLLDEVCRD

SEQ ID NO: 45 - protein, 537 aa, Arthrospira platensis
MELTELVELAEKERNNQKSIRIHCCTSTGCQASESLEVKKAMEDALKESGLEADVQIVGVGCMGFCGRGPMVEVEPNGIHYEKVTPEDAPSIIEALKGGEAKPVQGDPNHPFFSRQLKIVREFSGKIDPEKIGEYIAVGGYRALHHVIYDLTPEEVVAEITKSGLRGRGGAGYPTGLKWATVAKMPPGQKYVICNADEGDPGAFMDRSVLESDPHRILEGMAIAGYAVGATQGYIYVRAEYPLAITRLQKAIQQAKRQSMLGSRIFGSSINFNIDIRVGAGAFVCGEETALIASIGGGRGNPRPRPPYPAVSGLWDCPTLINNVETLGNIAPIIRNGADWYNQIGTEKSKGTKIFSLTGKIRNNGLIEVPMGITLREIVEEMGGGIPGGLQVKAVQTGGPSGGCIPAQFLDTPVDYESLTKLGSMMGSGGMVVMDENTSMVQVAQFYMEFCRGETCGKCIPCRVGTVQMYQLLTKLLNKQATPADLEKLKQLCDMVRATSLCGLGMTAPNPVTSTLQYFESEYLELLQTTNAKVAVH

SEQ ID NO: 46 - DNA, 1377 bp, Arabidopsis thaliana
ttcattcttcttctaaaacccgcaaaatttctccatttctaccaaaaatatccaacttttacttttctttcctgtgaaattatctgctcaaatctttggttcctgacggagatggcggcgatttcaggcatctcctctggtacgttgacgatttcacggcctttggttactcttcgacgctctagagccgccgtttcgtacagctcctctcaccgattgcttcatcatcttcctctctcttctcgtcgtctgctattaaggaacaatcatcgagtccaagcaacgattttgcaagacgatgaagagaaagtggtggtggaggaatcgtttaaagccgagacttctactggtacagaaccacttgaggagccaaatatgagttcttcttcaactagtgcttttgagacatggatcatcaagcttgagcaaggagtgaatgttttccttacagactcggttattaagatacttgacactttgtatcgtgaccgaacatatgcaaggttctttgttcttgagacaattgctagagtgccttattttgcgtttatgtctgtgctacatatgtatgagacctttggttggtggaggagagcagattatttgaaagtacactttgctgagagctggaatgaaatgcatcacttgctcataatggaagaattgggtggaaattcttggtggtttgatcgttttctggctcagcacatagcaaccttctactacttcatgacagtgttcttgtatatcttaagccctagaatggcatatcacttttcggaatgtgtggagagtcatgcatatgagacttatgataaatttctcaaggccagtggagaggagttgaagaatatgcctgcaccggatatcgcagtaaaatactatacgggaggtgacttgtacttatttgatgagttccaaacatcaagaactcccaatactcgaagaccagtaatagaaaatctatacgatgtgtttgtgaacataagagatgatgaagcagaacactgcaagacaatgagagcttgtcagactctaggcagtctgcgttctccacactccattttagaagatgatgatactgaagaagaatcagggtgtgttgttcctgaggaggctcattgcgaaggtattgtagactgcctcaagaaatccattacaagttaataaattagaaagtaaactaaaaaagattatttgtatcagctcatgaacaatagatataatcccatatacttgggaataaaggaataatgtgaaattcccatcgttgtgctagtgtgtgagagaatcaaataccctaatgatgtaaatgtactttgatgagcttaagtcgttgtagaccattttatccatactactagcctttgtggttgc

SEQ ID NO: 47 - protein, 351 aa, Arabidopsis thaliana
MAAISGISSGTLTISRPLVTLRRSRAAVSYSSSHRLLHHLPLSSRRLLLRNNHRVQATILQDDEEKVVVEESFKAETSTGTEPLEEPNMSSSSTSAFETWIIKLEQGVNVFLTDSVIKILDTLYRDRTYARFFVLETIARVPYFAFMSVLHMYETFGWWRRADYLKVHFAESWNEMHHLLIMEELGGNSWWFDRFLAQHIATFYYFMTVFLYILSPRMAYHFSECVESHAYETYDKFLKASGEELKNMPAPDIAVKYYTGGDLYLFDEFQTSRTPNTRRPVIENLYDVFVNIRDDEAEHCKTMRACQTLGSLRSPHSILEDDDTEEESGCVVPEEAHCEGIVDCLKKSITS

SEQ ID NO: 48 - protein, 238 aa, Synechocystis sp. PCC 6803
MSVVTLTIDDKAIAIEEGASILQAAKEAGVPIPTLCHLEGISEAAACRLCMVEVEGTNKLMPACVTAVSEEMVVHTNTEKLQNYRRMTVELLFSEGNHVCAICVANGNCELQDMAITVGMDHSRFKYQFPKREVDLSHPMFGIDHNRCILCTRCVRVCDEIEGAHVWDVAYRGAECKIVSGLNQPWGTVDACTSCGKCVDACPTGSIFHKGETTAEKIGDRRKVEFLATARKEKEWVR

SEQ ID NO: 49 - DNA, 744 bp, Synechococcus sp. WH 8102
atgaccgccaccacccccagcaaggagaccatcctggtggtggatgatgaggccagcatccgccgcatcctggagacccgcctgagcatgatcggctacaacgtggtgaccgcctgcgatggcaccgaggccctggagctgttcgagaacaccgcccccgatctggtggtgctggatgtgatgatgcccaagctggatggctacggcgtgtgccaggagctgcgcaaggagagcgatgtgcccatcgtgatgctgaccgccctgggcgatgtggccgatcgcatcaccggcctggagctgggcgccgatgattacgtggtgaagcccttcagccccaaggagctggaggcccgcatccgctgcgtgctgcgccgcgtggagaaggagagcgtggccggcatccccaacagcggcgtgatccaggtgagcgatctgcgcatcgataccaacaagcgccaggtgttccgcgccgatgagcgcatccgcctgaccggcatggagttcagcctgctggagctgctggtgagccgcagcggcgagcccttcaaccgcggcgagatcctgaaggaggtgtggggctacacccccgagcgccacgtggatacccgcgtggtggatgtgcacatcagccgcctgcgcagcaagctggaggatgatcccgccaaccccgagctgatcctgaccgcccgcggcaccggctacctgttccagcgcatcatcgatagcgtggccagcgagggcccc

SEQ ID NO: 50 - DNA, 699 bp, Tolypothrix sp. PCC 7601
atggtggatgatgaggccagcatccgccgcatcctggagacccgcctgagcatgatcggctacgatgtggtgaccgccggcgatggcgaggaggccctggagaccttccgcaaggccgatcccgatctggtggtgctggatgtgatgatgcccaagctggatggctacggcgtgtgccaggagctgcgcaaggagagcgatgtgcccatcatcatgctgaccgccctgggcgatgtggccgatcgcatcaccggcctggagctgggcgccgatgattacgtggtgaagcccttcagccccaaggagctggaggcccgcatccgcagcgtgctgcgccgcgtggataagaccagcgccagcggcatccccagcagcggcgtgatccacgtggccaacatcaagatcgataccaacaagcgccaggtgtacaagggcgatgagcgcatccgcctgaccggcatggagttcagcctgctggagctgctggtgagccgcagcggcgaggccttcagccgcagcgagatcctgcaggaggtgtggggctacacccccgagcgccacgtggatacccgcgtggtggatgtgcacatcagccgcctgcgcgccaagctggaggatgatcccagcaaccccgagctgatcctgaccgcccgcggcaccggctacctgttccagcgcatcatcgagcccggcgaggag

SEQ ID NO: 51 - DNA, 741 bp, Thermosynechococcus sp. NK55a
atggagacccacaaggagaagatcctggtggtggatgatgaggccagcatccgccgcatcctggagacccgcctgagcatgatcggctacaccgtggtgaccgccgccgatggcgaggaggccctgaccaccttccgccaggagcagcccgatctggtggtgctggatgtgatgatgcccaagctggatggctacggcgtgtgccaggagctgcgcaaggagagcgatgtgcccatcatcatgctgaccgccctgggcgatgtggccgatcgcatcaccggcctggagctgggcgccgatgattacgtggtgaagcccttcagccccaaggagctggaggcccgcatccgcagcgtgctgcgccgcatcgagaagaccaacaccagcggcatccccagcagcggcgtgatccaggtgggcaacatccgcatcgataccaacaagcgccaggtgtacaagggcgatgagcgcatccgcctgaccggcatggagttcatgctgctggagctgctggtgggccgcagcggcgagcccttcagccgcgccgagatcctggagcaggtgtggggctacacccccgagcgccacgtggatacccgcgtggtggatgtgcacatcagccgcctgcgcgccaagctggaggaggatcccagcaaccccgagctgatcctgaccgcccgcggcaccggctacctgttccagcgcatcaccgagcccggcgaggccagcaacaagaaccag

SEQ ID NO: 52 - DNA, 1746 bp, Synechococcus sp. WH 8103
atggtggtggccgccgccgcccccaagctgagcctgcagtgcgaggccatcgccggcgatacctgcaccatccgcagcctggattgggatcgcagccgcttcgatatcgagttcggcctgcgcaacggcaccacctacaacgccttcctggtgcgcggcgagcgcaccgccctgatcgataccagccacgccaagttccgcgatacctggatccccctgctgaaggagcagatcgagcccaccgccatcgatgtgctgatcgtgagccacaccgagcccgatcacagcggcctgatcggcgatctgatcgatctgaaccccgatatcgagatcgtgggcagcaaggtggccctgcagttcctgcaggatcaggtgcaccgccccttcaagagccgcgccgtgaagagcggcgaggagctggatctgggcaccaaccccgagagcggcatccagcaccgcttcgagttcctgagcgcccccaacctgcactggcccgataccatcttcagcttcgatcacggcaccggcatcctgtacacctgcgatgccttcggcctgcactactgcagcgatgatgtgttcgatagcgatcccggcgccatcgcccccgatttccgcttctactacgattgcctgatgggccccaacgcccgcagcgtgctgcaggccctgaagcgcatgggcggcctgcccgagatcaacaccatcgccgtgggccacggccccctgctgcgccaccacctgagccactgggtgaacgattaccgcgagtggagcggccagcgcagcaagggcgagagctacgccgccgtgtgctacctgagccagtacggcttcagcgatcgcatcagccaggccatcgcccacggcatcggcaaggccgaggcccaggtgcagctggtggatctgcgcgccaccgatccccaggagctgaccgccctgatcggcgatgccaaggccgtggtggtgcccacctggcccgccgagcccgatggcgagctgcaggccagcatcggcaccctgctggccgccctgcaccccaagcagctggtgggcgtgttcgatgccttcggcggcaacgatgagcccatcgatgccgtggccgatcagctgcgcagccagggccagaagcaggccttcgcccccctgcgcatccgccagctgccccagggcagcgattaccagcgctgcgaggagagcggcaccgatctgggccagctgctgaccaaggagaaggccatcgccgccatgaagagcctggatggcgatctggataaggccctgggccgcatcagcggcggcctgtacgtggtgaccgccagccagggcgagggcgagagccagcgccgcagcgccatggtggccagctgggtgagccaggccagcttcagcccccccggcctgaccgtggccgtggccaaggatcgcgccatcgaggccctgatgcaggtgggcgatcgcttcgtgctgaacgtgctgcgcgaggataaccaccagcccctgctgcgccacttcctgaagcgcttcccccccggcgccgatcgcttcgccggcgtgaacgtgctggagggcgtggccgatggcggccccgtgctgaccgatgccctggcctacctgagctgccgcgtggagcagcgcatggagggccccgatcactggatcatctacgccgtggtggagcagggcaacgtggccgatgccgaggccagcaccgccgtgcaccaccgcaaggtgggcaaccactac

SEQ ID NO: 53 - DNA, 1710 bp, Thermosynechococcus sp. NK55a
atgcccgtgaccaccaccaagcgccccccccgcctgaccctgcaggtgctggatatcgcccccgagaccaccgccatccgctgcctggattgggatcgcgatcgcttcgatatcgagttcgccctggagaacggcaccacctacaacagcttcctgatcaagggcgagcgcatcgccctggtggataccagccacgccaagttcggcgatcgctacctggatcagctgtggcagctggtgaaccccagcgatctgaactacctgatcgtgagccacaccgagcccgatcacagcggcctggtgaaggatgtgctgaccaaggccccccacgtgaccgtggtggccagcaaggtggccctgcagttcctgggcgatctgatccaccagcccttcagccagcagcaggtgaagaacggcgatcgcctggatctgggcaagggccacgtgctggagttcgtgatggcccccaacctgcactggcccgataccatcctgacctacgatcacggcacccagaccctgttcacctgcgatgtgttcggcgcccacttctgcaacgatgatcccttcgatagcgagcccgagctgctggcccccgatttcaagttctactacgattgcctgatgggccccaacgcccgcagcgtgctgagcgccttcaagcgcctggagcccctgccccccgtgcagctggtggccaccggccacggccccctgctgcgccaccacctggatcagtggctggagagctaccgcaactggagccaggagcaggccaaggccgccaccaccgtggccatcttctacgccgccaactacggctacagcaacgccctggccgaggccatcgagcgcggcgccgccaagaccggcgtggtggtggagaagatggatctgctgaccgccgagccccaggatatccgcgagctgaccgagatcgccgccggcatcatcatcggcaccccccccgccaccgccgtggcccagaccgccctgagcaccatccgcgccgccgcccacgccaagcaggccatcggcgtgttcgagagcgtggccgatgccgagcccgcctaccccctgctgaaccagttccgcgatgccggcctggtgcccagcttccccgtgatccaggtgaccgccacccccaccgatgccctgttccagcaggccgaggaggccggcaccgatatgggccagtggctgctgcgcgatcgcaccgtgaagcagatgaaggccctgaacaccgatctggataaggccctgggccgcctgagcggcggcctgtacatcatcaccgcccagaagggcgccatcaacagcgccatgctggccagctgggtggcccaggccagcaccgagcccctgggcgtgagcatcgccgtggccaaggatcgcgccatcgagagcttcctgcacgtgggcgataccttcgtgctgaacgtgctggaggagggcaactaccagcccctgatgcgccacttcctgaagcgcttcccccccggcgccgatcgcttcgcccacgtgaagacctaccccgccagcaacggcagccccatcctggccgatgccctggcctacctggagtgcaccgtggtgagccgcctggatgcccacgatcactggatcgtgtacagcaccgtggatagcggccgcgtgagcaagcccgatggcatgaccgccgtgcaccaccgcaaggtgggcaaccactac

SEQ ID NO: 54 - DNA, 1734 bp, Thermosynechococcus sp. NK55a
atgctgaccgagacccgcccccgcgatgtgcaggtggccgagatcgcccccggcatcctggtgctgcgcagccgcacctgggatcgcctgaagttcgaggtggagtacggccgccagcagggcaccaccagcaacagctacctgatccaggccccccagcccaccctgctggatccccccggcgagagcttcacccagatctacctgcaggagctgcagcagcacatcgatctgagccagctgcgctacctgatcctgagccacgtgaacagcaaccgcctggccaccgtgaaggtgctgctggagaaggccccccagatcaccatcgtgtgcagcaaggccgccgccgtggccctgcgcagcgccgtgggcgagcagctgaacctgtggatcccccgcgccgagacccccctggagctgggcggcgatcgccagctgatgttcatcgccgccgccaccccccgctggcccgatggcctgatgaccgtggatccccagaaccagatcgtgttcagcgataagctgttcggcgcccacgtgtgcggcgatagcctgtacgatgagcagtggaagaagctggatgaggatcgcgcctactacttcgagtgcctgcacgccgcccagacccgccaggtggagagcatcctggatcgcctggccgagctgaccccccccccccgcctgtacgcccccgcccacggccccatcgtgaagttcagccgcagccgcctgttccaggattaccgcgattggtgccaggcccagaccgcccaggataccaaggtggccctgttctacgccagcgcctacggcaacaccgccatcctggccaacgccatcgcccagggcctgaccgccgccggcgtgcaggtggaggccgtgaactgcgagaccaccccccccgccgagatgcaggccctgatccacagcagcgatggcttcatcatcggcagccccaccctgggcggccacatgcccacccaggtgcagaccgccctgggcttcatcctggccgagggcagccagaccaagctggccggcgtgttcggcagctacggctggagcggcgaggccatcgatgatatcgagcagaagctggtggatgccggctacaccctgggcttcgagaccctgcgcgtgaagttcacccccaccgccagcgatctggagaagtgccagatcgccgccagcgagttcgcccaggccctgaagaagctgcgcaagagccgcaccgtgcgcccccccagcctggccgaggcccaggtggatcgcaccgagcaggccgtgaaccgcgtggtgggcagcctgtgcgtgctgaccaccctgcccgagggctgcttccacctgacccaggccgccgccatcctggtgagcagcgtgagccaggccagcttccacccccccggcatcaccgtgagcctgccccaggagtgggccgagagcctgtgcctggtgggcgatcgcttcgtgctgaacatcctgaaggagggcagccccctggtgcgccagttccagcaggcccagcgcctgggcgagcagcagctggccaccctgggcctgaagagcgccgagagcggcgcccccatcctgctggatgccctggcctacctggagtgcaccgtggagagccgcatgaactgcggcaaccactggctgatctacgccgtggtggagagcggcgagctgctgcagaccagcggcctgaccgccatccagcaccgcaagaccagcagc

SEQ ID NO: 55 - DNA, 519 bp, Synechocystis sp. PCC 6803
atgaccgtggccaccgatcgccagaccgtgccccccagcgccgcccaccccagcggcgataagcgcttcaaggtgctggatgccaccatgaagcgcaaccagttcaaccaggatgccctgatcgagatcctgcacaaggcccaggagatcttcggctacctggaggaggatgtgctgctgtacgtggcccgcggcctgaagctgcccctgagccgcgtgttcggcgtggccaccttctaccacctgttcagcctgaagcccagcggcaagcacacctgcgtggtgtgcctgggcaccgcctgctacgtgaagggcgccggcgatctgctgaagaccctggatcaggaggtgcacctgaagcccggcgagaccaccgaggatggccagatgagcctggtgaccgcccgctgcatcggcgcctgcggcatcgcccccgccgtggtgtacgatggcaaggtgctgggcaagcagaacgatgaggccgtgctggccgccatccagccctggctgagcaacagc

SEQ ID NO: 56 - DNA, 552 bp, Synechococcus elongatus
atggataagatcaagctggccaccgtgtggctggccggctgcagcggctgccacatgagcttcctggatctggatgagtggctgatcgatctggccgatggcgtggaggtggtgttcagccccgtggcctgcgatcgcaaggattaccccgagggcgtggatctgtgcctgatcgagggcgccgtggccaacctggataacctggagctgctgcaccaggtgcgcgatcgcacccgcatcctggtgagcttcggcgattgcgccatccacgccaacgtgcccggcatgcgcaacctgtggggcgccgagagcgccgccgccgtgctggagcgcggctacctggagctggccgataccaccccccagctgcccaacgagcccggcatcgtgccccccctgctgaaccgcgtgacccccatccacgagctggtggccatcgagcactacctgcccggctgccccccccccgccgatcgcatccgcagcctgctgcaggccctgctggatcagcagacccccccccacagcggccgcgatctgctgaagttcggc

SEQ ID NO: 57 - DNA, 1422 bp, Cyanothece sp. ATCC 51142
atgagcaagaccatcgtgatcgatcccgtgacccgcatcgagggccacgccaagatcagcatcttcctggatgataacggccaggtggatgatgcccgcttccacgtggtggagtaccgcggcttcgagaagttctgcgagggccgccccatgtgggagatggccggcatcaccgcccgcatctgcggcatctgccccgtgagccacctgctggccagcgccaagaccggcgataagatcctggccgtgaagatcccccagcccgccgagaagctgcgccgcctgatgaacctggcccagatcacccagagccacgccctgagcttcttccacctgagcagccccgatttcctgctgggctgggatagcgatcccgccaagcgcaacatcttcggcctgatcgccagcgatcccgagctggccaagagcggcatccgcctgcgccagttcggccagaccatcatcgagctgctgggcgccaagaagatccacgccgcctggagcgtgcccggcggcgtgcgcagccccctgagcaaggatagccagcagtggatcaaggagcgcctgcccgaggcctacgagaccgccaacaacgccctgagcctgttcaagaagctgctggatcgcttcgagaccgaggtgaagaccttcggcgatttccccagcctgttcatgggcctggtgggcgaggatggcctgtgggagcactacggcggccacctgcgcttcagcgatagccagggcaacatcgtggccgatcacctgaaggaggaggattaccaggatttcatcggcgagagcgtggagaagtggagctacctgaagttcccctactacaagcccctgggctacccccagggcatctaccgcgtgggccccctggcccgcctgaacatctgcagccacttcggcaccgagaaggccgatcaggagctggaggagttccgcaaccgcgccggcggcgtggccaccagcagcttcttctaccactacgcccgcctggtggagatcatcggctgcctggagcgcatcgagcgcctgatgaacgatcccgatctgctgagcaccaacacccgcgccaaggccggcatcaacaacctggagggcatcggcgtgagcgaggccccccgcggcaccctgttccaccactacaacgtggatgagaacggcctgatcaagaaggtgaacctgatcatcgccaccggcaacaacaacctggccatgaacaagaccgtgacccagatcgccaagcactacgtgaagggcaacgccatgaccgagggcatgctgaaccgcgtggaggccggcatccgctgctacgatccctgcctgagctgcagcacccacgccgccggccagatgcccctgcacatccagctgttcggcgtgaacggcgatctgctggatgaggtgtgccgcgat

SEQ ID NO: 58 - DNA, 1611 bp, Arthrospira platensis
atggagctgaccgagctggtggagctggccgagaaggagcgcaacaaccagaagagcatccgcatccactgctgcaccagcaccggctgccaggccagcgagagcctggaggtgaagaaggccatggaggatgccctgaaggagagcggcctggaggccgatgtgcagatcgtgggcgtgggctgcatgggcttctgcggccgcggccccatggtggaggtggagcccaacggcatccactacgagaaggtgacccccgaggatgcccccagcatcatcgaggccctgaagggcggcgaggccaagcccgtgcagggcgatcccaaccaccccttcttcagccgccagctgaagatcgtgcgcgagttcagcggcaagatcgatcccgagaagatcggcgagtacatcgccgtgggcggctaccgcgccctgcaccacgtgatctacgatctgacccccgaggaggtggtggccgagatcaccaagagcggcctgcgcggccgcggcggcgccggctaccccaccggcctgaagtgggccaccgtggccaagatgccccccggccagaagtacgtgatctgcaacgccgatgagggcgatcccggcgccttcatggatcgcagcgtgctggagagcgatccccaccgcatcctggagggcatggccatcgccggctacgccgtgggcgccacccagggctacatctacgtgcgcgccgagtaccccctggccatcacccgcctgcagaaggccatccagcaggccaagcgccagagcatgctgggcagccgcatcttcggcagcagcatcaacttcaacatcgatatccgcgtgggcgccggcgccttcgtgtgcggcgaggagaccgccctgatcgccagcatcggcggcggccgcggcaacccccgcccccgccccccctaccccgccgtgagcggcctgtgggattgccccaccctgatcaacaacgtggagaccctgggcaacatcgcccccatcatccgcaacggcgccgattggtacaaccagatcggcaccgagaagagcaagggcaccaagatcttcagcctgaccggcaagatccgcaacaacggcctgatcgaggtgcccatgggcatcaccctgcgcgagatcgtggaggagatgggcggcggcatccccggcggcctgcaggtgaaggccgtgcagaccggcggccccagcggcggctgcatccccgcccagttcctggatacccccgtggattacgagagcctgaccaagctgggcagcatgatgggcagcggcggcatggtggtgatggatgagaacaccagcatggtgcaggtggcccagttctacatggagttctgccgcggcgagacctgcggcaagtgcatcccctgccgcgtgggcaccgtgcagatgtaccagctgctgaccaagctgctgaacaagcaggccacccccgccgatctggagaagctgaagcagctgtgcgatatggtgcgcgccaccagcctgtgcggcctgggcatgaccgcccccaaccccgtgaccagcaccctgcagtacttcgagagcgagtacctggagctgctgcagaccaccaacgccaaggtggccgtgcac

SEQ ID NO: 59 - DNA, 714 bp, Synechocystitis sp. PCC6803
atgagcgtggtgaccctgaccatcgatgataaggccatcgccatcgaggagggcgccagcatcctgcaggccgccaaggaggccggcgtgcccatccccaccctgtgccacctggagggcatcagcgaggccgccgcctgccgcctgtgcatggtggaggtggagggcaccaacaagctgatgcccgcctgcgtgaccgccgtgagcgaggagatggtggtgcacaccaacaccgagaagctgcagaactaccgccgcatgaccgtggagctgctgttcagcgagggcaaccacgtgtgcgccatctgcgtggccaacggcaactgcgagctgcaggatatggccatcaccgtgggcatggatcacagccgcttcaagtaccagttccccaagcgcgaggtggatctgagccaccccatgttcggcatcgatcacaaccgctgcatcctgtgcacccgctgcgtgcgcgtgtgcgatgagatcgagggcgcccacgtgtgggatgtggcctaccgcggcgccgagtgcaagatcgtgagcggcctgaaccagccctggggcaccgtggatgcctgcaccagctgcggcaagtgcgtggatgcctgccccaccggcagcatcttccacaagggcgagaccaccgccgagaagatcggcgatcgccgcaaggtggagttcctggccaccgcccgcaaggagaaggagtgggtgcgc

(19) United States
(12) Patent Application Publication
(10) Pub. No.: US 2019/0062763 A1
(43) Pub. Date: Feb. 28, 2019

(54) MICROORGANISMS WITH NADPH ESCAPE VALVES TO PROVIDE REDUCED PHOTODAMAGE AND INCREASED GROWTH IN HIGH LIGHT CONDITIONS

(71) Applicants: LUMEN BIOSCIENCE, INC., Seattle, WA (US); RELIANCE HOLDING USA, INC., Houston, TX (US)

(72) Inventors: James ROBERTS, Seattle, WA (US); Damian CARRIERI, Seattle, WA (US); Mark HEINNICKEL, Seattle, WA (US)

(21) Appl. No.: 16/076,788

(22) PCT Filed: Feb. 10, 2017
(86) PCT No.: PCT/US17/17539

§ 371 Date: Aug. 9, 2018

Related U.S. Application Data
(60) Provisional application No. 62/294,895, filed on Feb. 12, 2016.
(60) Provisional application No. 62/404,547, filed on Oct. 5, 2016.

(51) Int. Cl. C12N 15/74; C07K 14/195

(52) U.S. Cl. CPC C12N 15/74; C07K 14/195

(57) ABSTRACT

Microorganisms with NADPH escape valves are described. The NADPH escape valves convert NADPH to NADP and decrease photodamage and/or increase ATP production, growth, and/or biomass yield in high light conditions. NADPH escape valves can be created by up-regulating enzymes or other molecules that convert NADPH to NADPH (e.g., transhydrogenases, Flv3, Flv1, hox hydrogenase, and/or PTOX).

### CROSS-REFERENCE TO RELATED APPLICATIONS

This application claims priority to U.S. Provisional Patent Application No. 62/294,895 filed on Feb. 12, 2016 and to U.S. Provisional Patent Application No. 62/404,547 filed on Oct. 5, 2016, both of which are incorporated herein by reference in their entirety as if fully set forth herein.

### REFERENCE TO SEQUENCE LISTING

A computer readable text file, entitled “DN1MC8371.txt (Sequence Listing.txt)” created on or about Feb. 7, 2017, with a file size of 93.0 KB, contains the sequence listing for this application and is hereby incorporated by reference in its entirety.

### FIELD OF THE DISCLOSURE

The disclosure provides microorganisms with NADPH escape valves. The NADPH escape valves convert NADPH to NADP and reduce photodamage and/or increase growth, and/or biomass yield in high light conditions. NADPH escape valves can be created by up-regulating enzymes or other molecules that convert NADPH to NADP (e.g., transhydrogenases, Flv3, Flv1, hox hydrogenase, and/or PTOX).

### BACKGROUND OF THE DISCLOSURE

Photosynthesis is a process by which solar energy is converted into chemical bond energy. The process of photosynthesis ultimately results in biomass accumulation. Biomass can be used to produce energy, fuel, chemicals, and food. As examples, bioethanol can be produced through alcohol fermentation of saccharified carbohydrate, and biodiesel oil and biojetfuel can be produced from neutral lipids such as waxesters and triglycerides. Further, photosynthesis processes environmental carbon dioxide.

Photosynthetic crops such as soy beans, corn, and palms have been used as raw materials to produce biofuel and other products. Use of edible crops for such purposes, however, can contribute to food shortages. Non-edible crops such as jatropha and camelina have also been used, but these crops have low yields per unit area.

Photosynthetic microorganisms similarly can be cultivated to produce energy, fuel, chemicals, and food, as well as to process environmental carbon dioxide. In fact, many of these photosynthetic microorganisms are capable of producing larger amount of oils, fats and carbohydrates than plants.

### SUMMARY OF THE DISCLOSURE

When photosynthetic organisms experience high light they can suffer an imbalance between the energy generated by light harvesting and their ability to use that energy for carbon fixation. It is often said that these cells become “over-reduced.”

Light energy is used to make both ATP and metabolic reducing power (NADPH) during “light reactions”. When more NADPH is made than can be used to fix carbon, the NADPH pool becomes saturated and the excess energy must be dissipated in some manner, often as high energy electrons that cause photodamage.

The disclosure provides microorganisms with NADPH escape valves. In particular embodiments, the NADPH escape valves convert NADPH to NADP and reduce photodamage, and/or increase growth, and/or biomass yield in high light conditions. NADPH escape valves can be created by up-regulating enzymes or other molecules that convert NADPH to NADP (e.g., transhydrogenases, Flv3). NADPH escape valves can also be created by facilitating transfer of electrons from NADPH to plastoquinone to produce plastoquinol. Microorganisms with NADPH escape valves can also optionally have down-regulated RpaB pathway activity.

In particular embodiments, photosynthetic microorganisms are modified to up-regulate a protein complex, transhydrogenase, which catalyzes the interconversion of the reduction state of the intracellular pyridine nucleotides Nicotinamide adenine dinucleotide phosphate (NADPH) and Nicotinamide adenine dinucleotide (NADH) per the reaction below:

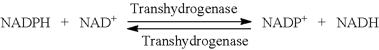

In particular embodiments, photosynthetic microorganisms are modified to up-regulate PTOX protein which facilitates the transfer of electrons from NADPH to plastoquinone to produce plastoquinol.

### BRIEF DESCRIPTION OF THE FIGURES

FIG. 1 depicts a schematic of a mechanism supporting increased ATP and carbon fixation through “NADPH escape valves” created by transhydrogenase up-regulation.

FIG. 2 depicts the plasmid map of pMX1137 used to make strains MX2134 and MX2135.

FIGS. 3A and 3B depict DNA (3A; SEQ ID NO: 33) and protein (3B; SEQ ID NO: 34) sequences of Synpcc7942_1610.

FIGS. 4A and 4B depict DNA (4A; SEQ ID NO: 35) and Protein (4B; SEQ ID NO: 36) sequences of Synpcc7942_1611.

FIGS. 5A and 5B depict DNA (5A; SEQ ID NO: 37) and Protein (5B; SEQ ID NO: 38) sequences of Synpcc7942_1612.

FIGS. 6A-6C depict growth curve (6A) of S7942 strains grown under 1000 μE*m⁻²*s⁻¹ continuous illumination at 30° C. under a gaseous atmosphere of 1% CO₂ enriched air as measured by optical density (OD750) and dry biomass yields (Dry Weights) after 48 (6B) and 72 (6C) hours of growth. Data are averages of three biological replicates. Error bars represent 1 standard deviation.

FIG. 7A-7C depict growth curve (7A) of S7942 strains grown under 1000 μE*m⁻²*s⁻¹ continuous illumination at 30° C. under a gaseous atmosphere of 1% CO₂ enriched air as measured by optical density (OD750) and dry biomass yields (Dry Weights) after 53 (7B) and 72 (7C) hours of growth. Data are averages of three biological replicates. Error bars represent 1 standard deviation.

FIG. 8 depicts relative mRNA expression levels of hliA in wild type (e.g., non-modified) and mutant strain TJ132≡MX1296 (Ptrc:N-rpaB) without (U) and with (I) addition of 1 mM IPTG.

FIG. 9 depicts total photosystem II (PSII) activity of wild type and strain MX1296 grown without or with 1 mM IPTG in medium. Activity was measured by determining the rate of oxygen evolution of whole cells in the presence of para-benziquinone and potassium ferricyanide, which serve to accept electrons directly from PSII, allowing for PSII oxygen evolution to run at maximal rate, independent of down-stream proteins in the electron transport chain.

FIG. 10 depicts total electron transport chain activity of wild type and strain MX1296 grown with 1 mM IPTG in medium. Activity was measured by determining the rate of oxygen uptake of whole cells in the presence of methyl viologen and potassium cyanide, which serve to accept electrons directly from photosystem I (PSI), allowing for the entire electron transport chain to run at maximal rate, independent of down-stream proteins in, e.g., carbon fixation or nitrate reduction.

FIG. 11 depicts the plasmid map of pMX1317 used to make strains MX2349.

FIGS. 12A and 12B depict DNA (12A; SEQ ID NO: 46) and protein (12B; SEQ ID NO: 47) primary sequences of At4G22260.

FIG. 13 depicts initial growth experiments under 900 μE*m⁻²*s⁻¹ continuous illumination flux and 1% CO₂/air atmosphere showed improvements in growth rates and yields of the At-PTOX overexpressing strain (MX2394) relative to respective WT control. Strains were either induced with 1 mM IPTG (At-PTOX_I) or analyzed without external induction (At-PTOX_U). Western blot was performed with a commercial antibody (From Uniplastomic, Catalogue number AB012) showing the effect of IPTG on protein levels. Note that At-PTOX induced expresses PTOX protein at a high level, and At-PTOX uninduced expresses PTOX protein at a lower level. This lower level is sufficient to cause a useful growth phenotype. WT cells express no PTOX protein.

FIG. 14 depicts dry weights taken 72 hours after inoculation. Strains were either induced with 1 mM IPTG (At-PTOX_I) or analyzed without external induction (At-PTOX_I). Intracellular PTOX levels for PTOX expressing strains under these IPTG concentrations are shown in FIG. 13 (inset).

FIG. 15 depicts alterations in oxygen evolution after the application of 5 minute periods of illumination (x-axis). The WT is less resistant to these illumination periods than the PTOX expressing strains. Strains were either induced with 1 mM IPTG (At-PTOX_I) or analyzed without external induction (At-PTOX_U). Intracellular PTOX levels for PTOX expressing strains under these IPTG concentrations are shown in FIG. 13 (inset).

FIG. 16 depicts rate of oxygen evolution of WT and PTOX expressing strains at increasing light intensities. Strains were re-suspended in BG11+20 mM Na₂HPO₄ (pH 7.2)+10 mM NaHCO₃. Strains were either induced with 1 mM IPTG (At-PTOX_I) or analyzed without external induction (At-PTOX_U). Intracellular PTOX levels for PTOX expressing strains under these IPTG concentrations are shown in FIG. 13 (inset).

FIG. 17 depicts initial growth experiments under 250 μE*m⁻²*s⁻¹ continuous illumination flux and 1% CO₂/air atmosphere showed improvements in growth rates and yields of the At-PTOX overexpressing strain relative to respective WT control at 26 C (a non-optimum temperature for this strain). Strains were either induced with 1 mM IPTG (At-PTOX_I) or analyzed without external induction (At-PTOX_U). Intracellular PTOX levels for PTOX expressing strains under these IPTG concentrations are shown in FIG. 13 (inset).

FIG. 18 depicts additional exemplary sequences referenced within the disclosure (SEQ ID NOs: 1-32, 39-45 and 48-59).

### DETAILED DESCRIPTION

Photosynthesis is a process by which solar energy is converted into chemical bond energy. The overall reaction of photosynthesis is the light-driven conversion of carbon dioxide and water to glucose and oxygen:

6CO₂+6H₂O→C₆H₁₂O₆+6O₂

Photosynthesis is observed in plants as well as in bacteria, including blue-green algae.

The process of photosynthesis ultimately results in biomass accumulation. Biomass can be used to produce energy, fuel, chemicals, and food. As examples, bioethanol can be produced through alcohol fermentation of saccharified carbohydrate, and biodiesel oil and biojetfuel can be produced from neutral lipids such as waxesters and triglycerides. Further, photosynthesis processes environmental carbon dioxide.

Photosynthesis includes two stages called the light reactions and the dark reactions. The light reactions require the presence of light, while the dark reactions do not depend on direct light exposure. In the light reactions, sunlight is absorbed and drives an electron transport chain that results in the formation of the energy carriers NADPH and ATP, forming O₂ as a by-product. In the dark reactions, a reaction driven by NADPH and ATP reduces CO₂ to glucose.

Photosystems are large multiprotein complexes that allow, in collaboration with other components, the conversion of captured solar energy into chemical bond energy via the electron transport chain. In general, photosystems are made up of two components: (1) a photochemical reaction center that allows solar energy to be converted into chemical energy, and (2) an antenna complex which captures light energy and transfers it to the photochemical reaction center, resulting in excitation of the photosystem.

The source of electron replenishment in a photosynthesis system differs according to the reaction center type. In purple non-sulfur bacteria, for example, electrons are cycled back to the reaction center by water-soluble electron carriers, for example, a cytochrome c type protein. In oxygenic photosynthetic organisms, including Cyanobacteria, red and green algae and plants, electron flow is non-cyclic, and occurs in two steps that involve two photosystems: Photosystem I (PSI) and Photosystem II (PSII). In these types of reactions, the deficit of electrons can be replenished by electrons taken from water molecules.

PSII is a complex composed of proteins, pigments and cofactors, located within thylakoid membranes. PSII splits water into oxygen, protons and electrons. Oxygen is released into the atmosphere and is responsible for maintaining aerobic life on Earth. The electrons are immediately energized by a photon (λ=680 nm) in PSII and passed from one compound to another, all of which compose the electron transport chain. Most of the electron carriers are quinones (Q), plastiquinones (PQ), or cytochromes (Cyt).

More particularly, the process of electron transfer in PSII includes the following steps: upon illumination, a P₆₈₀ chlorophyll is photoexcited. The photoexcited P₆₈₀ transfers electrons via intermediate cofactors called pheophytin a and plastoquinone A (PQ, Q_{A}) in order to finally doubly reduce a transiently bound PQ molecule (Q_{B}). Q_{B}²⁻ is protonated and released from the reaction center into the thylakoid membrane. The redox active cofactors that enable electron transfer from water to the secondary quinone acceptor Q_{B} are mainly embedded within two proteins called D1 and D2. Under normal conditions of illumination, the D1 protein of the reaction center core is irreversibly damaged over time and is replaced in a fashion that preserves the integrity of the PSII complex.

A second input of light energy (λ=700 nm) occurs during PSI and the energized electrons are passed to the terminal electron carrier, ferredoxin (Fd). Reduced Fd can serve as an electron donor to the ferredoxin-NADP⁺-reductase (FNR) enzyme. In a parallel process (photophosphorylation), H⁺ are released where they generate a H⁺ gradient that is used to drive ATP production via ATP synthase. NADPH and ATP are subsequently used to produce starch and other forms of energy storage biomass.

Cyanobacteria are the only group of organisms that are able to reduce nitrogen and carbon in aerobic conditions. The water-oxidizing photosynthesis is accomplished by coupling the activity of PSII and PSI (the Z-scheme). In anaerobic conditions, Cyanobacteria are also able to use only PSI (i.e., cyclic photophosphorylation) with electron donors other than water (e.g., hydrogen sulfide, thiosulphate, or molecular hydrogen), similar to purple photosynthetic bacteria. Furthermore, Cyanobacteria share an archaeal property—the ability to reduce elemental sulfur by anaerobic respiration in the dark. The Cyanobacterial photosynthetic electron transport system shares the same compartment as the components of respiratory electron transport. Typically, the plasma membrane contains only components of the respiratory chain, while the thylakoid membrane hosts both respiratory and photosynthetic electron transport.

Phycobilisomes are complexes of phycobiliproteins and colorless polypeptides which function as the major light harvesting antennae in blue-green and red algae. The phycobilisome components (phycobiliproteins) are responsible for the blue-green pigmentation of most Cyanobacteria. Color variations are mainly due to carotenoids and phycoerythrins, which may provide the cells with a red-brownish coloration. In some Cyanobacteria, the color of light influences the composition of phycobilisomes. In green light, the cells accumulate more phycoerythrin, whereas in red light they produce more phycocyanin. Thus, the bacteria appear green in red light and red in green light. This process is known as complementary chromatic adaptation and represents a way for the cells to maximize the use of available light for photosynthesis.

Photosynthetic organisms must cope with environmental changes in their habitats, such as fluctuations in incident light. When photosynthetic organisms experience high light they can suffer an imbalance between the energy generated by light harvesting and their ability to use that energy for carbon fixation. It is often said that these cells become “over-reduced.” As stated, light energy is used to make both ATP and metabolic reducing power (NADPH) during the light reactions. When more NADPH is made than can be used to fix carbon, the NADPH pool becomes saturated and the excess energy must be dissipated in some manner, often as high energy electrons that cause photodamage. The present disclosure provides NADPH escape valves that can reduce photodamage and increase growth, and/or biomass yield in high light conditions and/or conditions where temperature is lower than optimum.

In particular embodiments, the NADPH escape valves convert NADPH to NADP. NADPH escape valves can be created by up-regulating enzymes or other molecules that convert NADPH to NADP (e.g., transhydrogenases). In particular embodiments, NADPH escape valves transfer electrons from NADPH to plastoquinone to produce plastoquinol by the Ndh complex (PTOX). Microorganisms with NADPH escape valves can also optionally have down-regulated RpaB pathway activity.

In particular embodiments, photosynthetic microorganisms are modified to up-regulate NADHP escape valves, such as (i) the protein complex, transhydrogenase, (ii) Flv3, (iii) Flv3 in combination with Flv1; (iv) hox hydrogenase; and/or (v) PTOX.

Transhydrogenases catalyze the interconversion of the reduction state of the intracellular pyridine nucleotides Nicotinamide adenine dinucleotide phosphate (NADPH) and Nicotinamide adenine dinucleotide (NADH) per the reaction below:

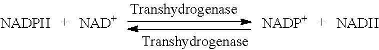

In particular embodiments, transhydrogenases incluse those enzymes that correspond to Enzyme Commission No. 1.6.1.1.

Flv3, another NADPH escape valve converts NADPH as follows:

NADPH+O₂->NADP+H₂O

Additionally, Flv3 and Flv1 form a heterodimer in Synechocystis sp. PCC 6803 to function as an NADPH escape valve. Thus, Flv3 alone can be used as an NADPH escape valve as disclosed herein, as well as Flv3 in combination with Flv1.

Hox hydrogenases including E, F, U, Y, and H proteins (see, e.g., Carrieri et al., Bioresource Technology 102 (2011) 8368-8377) also provide NADPH escape valves.

In particular embodiments, overexpression of a three gene operon encoding the subunits of a pyridine nucleotide transhydrogenase confers reduced photodamage, and/or increased ATP production, growth, and/or biomass yield in high light conditions. The Synpcc7942_1610 gene encodes a transhydrogenase beta subunit, and the Synpcc7942_1612 gene encodes a transhydrogenase alpha subunit. The beta subunit and the alpha subunit come together as a protein complex. Synpcc7942_1610 and Synpcc7942_1612 are homologous to bacterial membrane bound transhydrogenases encoded by genes pntB and pntA, which are usually in an operon as pntAB. These transhydrogenases are distinct from the non-membrane bound transhydrogenases encoded by the udhA gene in E. coli. The Synpcc7942_1611 gene encodes an “alpha subunit-like” gene. In particular embodiments, Synpcc7942_1610, Synpcc7942_1612, and Synpcc7942_1611 are expressed as one transcript.

Without being bound by theory, it is believed that up-regulation of transhydrogenase increases catalytic activity for the interconversion of reduced NADPH and NADH allowing for better metabolic flexibility between NADPH, a reduction cofactor involved in photosynthetic anabolism, and NADH, a reduction cofactor involved in respiration and ATP (energy) generation under high light fluxes. Thus more electrons can be extracted from water via photosynthesis and directed to growth and biomass accumulation and avoid damaging reactions that cause photodamage. Stated another way, up-regulation of NADPH to NADP converting enzymes or other molecules (e.g., transhydrogenases) provides an “escape valve” or “electron valve” by converting NADPH to NADH. The NADPH pools thus remain unsaturated, and damage from high energy electrons is diminished. Moreover, the NADH can be used for intermediary metabolism, and also to produce additional ATP.

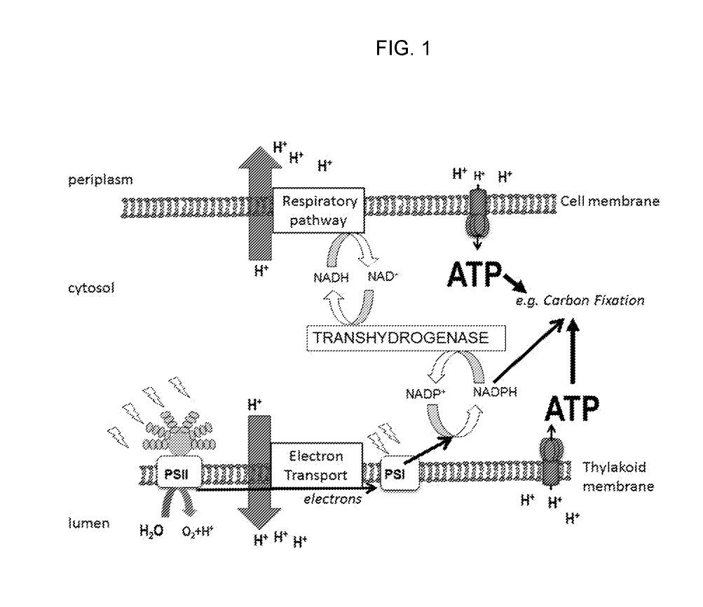

Regarding production of additional ATP, and again without being bound by theory, allowing cells to convert NADPH (a product of photosynthesis) to NADH (a product of catabolic metabolism that feeds into respiration), also allows the cells to use some of this reductant for making extra ATP to boost carbon fixation and biomass accumulation. As stated, under high light conditions, cells can make excess NADPH. By directing this NADPH into respiration, and therefore ATP production, there is a boost in energy at the expense of “extra” NADPH being burned off. By allowing for NADPH to convert to NADH, protons can be pumped across the cell membrane, allowing for added ATP production. See, for example, FIG. 1. In particular embodiments, because the transhydrogenase is a membrane bound transhydrogenase, it will not only use NADH (product of transhydrogenation) for respiration, but also will provide an ATP boost from the energy translocation of the interconversion itself.

As indicated, up-regulation of PTOX also provides an NADPH escape valve that confers improved growth rates and yields under high light and low temperature growth conditions. Without being bound by theory, the valve, in this case, involves the transfer of electrons from NADPH to plastoquinone to produce plastoquinol by the Ndh complex. This is then linked to the oxidation of plastoquinol by PTOX, and consequent reduction of molecular oxygen to water. The net reaction is oxidation of NADPH and reduction of oxygen.

This pathway allows for the oxidation of NADPH in high light, a condition in which NADPH is in excess and in much higher concentration than NADP⁺. When the NADPH/NADP⁺ ratio is high photodamage can occur that affects Photosystem I. Thus, by oxidizing the plastoquinone pool, PTOX provides a valve for NADPH when it accumulates to dangerous levels (with the ultimate destination for these electrons being oxygen). In this sense, it provides an outlet similar to transhydrogenase or Flv3.

Oxidation of plastoquinol by PTOX may have another beneficial consequence. Plastoquinone, the oxidized form of plastoquinol, is a redox active cofactor that is dissolved in photosynthetic membranes (thylakoids) in plants and algae. Plastoquinone acts as a redox active cofactor that is reduced by many important enzymes including photosystem II and the Ndh complex. Photosystem II (PSII), the enzyme responsible for water oxidation, can accumulate damage (termed photodamage) when there is a lack of plastoquinone molecules that are available for reduction. The most common reason for the absence of plastoquinone is that the “pool” of these molecules is mostly reduced to the plastoquinol state, which cannot accept further electrons from PSII. Again without being bound by theory, overexpression of PTOX oxidizes plastoquinols before the “pool” is reduced at a large enough fraction to prevent electron transfer from PSII. PTOX activity thereby prevents photodamage to PSII, which can lower photosynthesis and growth rates in plants, algae and cyanobacteria.

Before the current disclosure, it could not be anticipated whether NADPH escape valve up-regulation could reduce photodamage and/or stimulate growth and biomass yield in high light conditions. The major limitation to high light growth is photodamage, and it could not be predicted that increased ATP levels would, per se, be sufficient to mitigate photodamage. In fact, one might anticipate that increased ATP alone would not be sufficient, because this would not reduce the destruction/inactivation of photosystem proteins by high energy electrons that is responsible for photodamage in high light. It was speculated that relieving the backup of the NADPH pool in high light conditions might prevent accumulation of high energy electrons and consequent photodamage. But it was unknown whether up-regulation of an escape valve would indeed reduce NADPH levels sufficiently to have this desired effect. In fact, Hasunma et al. (Biotechnology for Biofuels (2014) 7:493) measured NADPH levels in escape valve Flv3 overexpressing cells in low light, and did not see any difference compared to control cells. Moreover, transhydrogenase up-regulated cells do not show increased growth in low light, and Flv3 up-regulated cells show only a small increase in growth in low light (defined here as 120 uE/m²s photon flux), a much smaller magnitude than that seen in high light conditions, as disclosed herein. See Hasunma et al., supra.

Morever, and regarding the PTOX NADPH escape valve, reports in the literature show that PTOX does not help the growth of certain photosynthetic organisms. For example, in tobacco, the overexpression of PTOX did oxidize the plastoquinone pool in high light, but it did not increase growth rates. Further, although PTOX is believed to have some role in stress alleviation, there are no reports indicating that its overexpression would aid growth in low temperature growth conditions. This finding was serendipitous and indicates that this stress reduction protein may also have a role in other situations when photodamage accumulates. See, for example, et al., J Biol Chem. (2002) 277:31623-30.

In particular embodiments, modified photosynthetic microorganisms disclosed herein show reduced photodamage in high light conditions. Reduced photodamage can be observed through protection against destruction of photosystem proteins by high energy electrons and reactive oxygen species. Reduced photodamage can be measured through methods including Western blot analysis of, for example, PsaA, PsaC, PsaD, PsaE (Photosystem I); PsbA, PsbB (Phototsystem II); PetF (ferredoxin); and/or PetH (FNR). In particular embodiments, reduced photodamage is a statistically significant reduction in damage to one or more of PsaA, PsbB, PsaC, PsaD, PsaE, PsbA, PsbB, PetF and PetH in high light conditions.

Reduced photodamage can also be observed through conservation of photosystem function. Conserved photosystem function can be observed through biochemical assays that measure maximum activity of Photosystems I and II. Photosystem I assays measure maximum activity of Photosystem I by providing saturating amounts of chemical substrates ascorbic acid and methyl viologen for oxidation and reduction, respectively, by Photosystem I. Reduced methyl viologen subsequently reduces molecular oxygen to hydrogen peroxide, and the consumption rate of oxygen is related to the maximum activity rate of Photosystem I. Photosystem II assays measure maximum activity of Photosystem II by providing saturating amounts of chemical substrates water and para-benzoquinone for oxidation and reduction, respectively, by Photosystem II. Para-benzoquinone is subsequently oxidized by a terminal chemical electron acceptor, potassium ferricyanide. Oxidation of water produces molecular oxygen, and the production rate of molecular oxygen is related to the maximum activity rate of Photosystem II. In particular embodiments, reduced photodamage is a statistically significant conservation of photosystem I and II function in high light conditions.

In particular embodiments, modified photosynthetic microorganisms disclosed herein show increased growth in high light conditions and/or low temperature growth conditions. Increased growth can be confirmed by growth curve analysis of strains grown under 1000 μE*m⁻²*s⁻¹ continuous illumination at 30° C. under a gaseous atmosphere of 1% CO₂ enriched air as measured by optical density (OD750). In particular embodiments, increased growth is a statistically significant increase in growth.

In particular embodiments, modified photosynthetic microorganisms disclosed herein show increased biomass yields in high light conditions and/or low temperature growth conditions. Increased biomass yields can be confirmed by the weights of dry biomass yields after 72 hours of growth. In particular embodiments, increased biomass yields can be a statistically significant increase in biomass yields.

In particular embodiments, modified photosynthetic microorganisms disclosed herein show increased ATP production in high light conditions. Increased ATP production has many benefits including providing resistance to many stresses and increased production of specific metabolic products. In particular embodiments, increased ATP in cells can more precisely be defined as an increase in the cellular energy charge for the microorganism. From classical biochemistry, the energy charge of a cell is defined by an index that ranges from 0 to 1:

$$Energy charge = \frac{[ ATP ] + \frac{1}{2} [ ADP ]}{[ ATP ] + \{ ADP ] + [ AMP ]}$$

This parameter can be determined by measuring the intracellular concentrations of ATP, ADP, and AMP in a culture and calculating the index value. An exemplary method to measure these concentrations is liquid chromatography coupled to mass spectrometry. In particular embodiments, increased ATP production is a statistically significant increase in ATP production.

High light conditions include photon fluxes above 350 microE m⁻² s⁻¹ (350 micromoles photons per m² per second).

Low temperature growth conditions or lower than optimum growth conditions are those where the growth temperature is at least 5° C. below the optimum growth temperature of a species. The optimum growth temperature of a particular species can be determined experimentally using methods well known to those of ordinary skill in the art. In practice, all outdoor cultures naturally go through wide temperature fluctuations during any typical day/night cycle such that most, if not all, outdoor cultures experience at least for some period of time temperatures that are at least 5° C. below their optimum growth temperature. For particular strains, the optimum growth temperature is 30° C.

Changes in light quantity or quality (i.e., spectral composition) can also result in imbalanced excitation of PSII and PSI and decrease the efficiency of photosynthetic light reactions. Photosynthetic organisms can counteract such excitation imbalances with changes in gene expression.

OmpR response regulators are response regulators wherein their phosphorylation promotes specific DNA binding by enhancing dimer or oligomer formation. In some cases, dephosphorylated OmpR response regulators have >10-fold lower affinity to their binding sites than phosphorylated forms. RpaA and RpaB are two types of OmpR response regulators.

The NbIS-RpaB signaling pathway is the most conserved two-component system in Cyanobacteria. This pathway is involved in regulation of circadian-based changes in gene expression, regulation of photosynthesis, and acclimatization to a variety of environmental conditions.

The full length protein RpaB has an N-terminal phospho-reciever domain and a C-terminal DNA binding domain. The C-terminal domain is responsible for binding promoter regions such as HLR1 (high light-responsive element 1) and repressing transcription of downstream genes when RpaB is phosphorylated (at the N-terminal side of the protein). N-terminal fragments of the RpaB protein can have a phospho-receiver domain but no known DNA binding domain. The phosphorylatable residue of RpaB is thought to be Asp56.

With regard to regulation of circadian-based changes in gene expression, RpaB binds the KaiBC promoter and represses transcription of kaiBC and other target genes during subjective night (e.g., ˜LL0). During subjective day (e.g., ˜LL4-8), RpaB is released from these promoters, likely through the effects of RpaA, to allow transcription of the repressed genes.

As stated, with regard to regulation of photosynthesis, RpaB binds to the promoter HLR1. The HLR1 motif (SEQ ID NO: 1) includes two direct repeats (G/T)TTACA(T/A)(T/A) separated by two nucleotides (n₂). When bound to HLR1, RpaB represses transcription of genes, such as rpoD3 and hIiA. Under high light stress, RpaB is dephosphorylated in a process mediated by NbIS to allow translation of these genes.

Decreasing the copy number of RpaB genes in Cyanobacteria decreases energy transfer from phycobilisomes to PSII and increases energy transfer from phycobilisomes to PSI. Thus, it is been suggested that RpaA and RpaB regulate expression of proteins involved in the coupling of phycobilisomes to PSI or PSII. With regard to acclimation to other environmental conditions, RpaB has been shown to modulate transcription of genes in response to cold shock as well as osmotic, salt and oxidative stresses. In spite of the importance of the NbIS-RpaB signaling pathway, actual input signals and output responses remain largely unknown.

The current disclosure provides microorganisms with decreased photodamage and/or increased ATP production, growth, and biomass yield. In addition to up-regulating NADPH escape valves (e.g., transhydrogenases, PTOX), particular embodiments also include down-regulating activity of the RpaB pathway. Exemplary methods to up-regulate NADPH escape valves or optionally down-regulate activity of the RpaB pathway are described more fully elsewhere herein.

Photosynthetic Microorganisms. Photosynthetic microorganisms of the disclosure may be any type of organism capable of performing photosynthesis wherein the microorganism has been modified to have at least one up-regulated NADPH escape valve (e.g., transhydrogenase, PTOX) and optionally, down-regulated RpaB pathway activity.

Exemplary photosynthetic microorganisms that are either naturally photosynthetic or can be engineered to be photosynthetic include bacteria (e.g., Cyanobacteria); fungi; archaea; protists; eukaryotes, such as a green algae; and animals such as plankton, planarian, and amoeba. Examples of naturally occurring photosynthetic microorganisms include Arthrospira (Spirulina) maxima, Arthrospira (Spirulina) platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechoccus sp., Synechocystis sp., Cyanobacterium aponinum, and Tolypothrix sp.

Cyanobacteria, also known as blue-green algae, blue-green bacteria, or Cyanophyta, is a phylum of bacteria that obtain their energy through photosynthesis. As stated, Cyanobacteria can produce metabolites, such as carbohydrates, proteins, lipids and nucleic acids, from CO₂, water, inorganic salts and light. Any Cyanobacteria may be used according to the disclosure. In particular embodiments the Cyanobacteria must be genetically manipulatable, e.g., permissible to the introduction and expression of exogenous genetic material (e.g., exogenous nucleotide sequences).

Cyanobacteria include both unicellular and colonial species. Colonies may form filaments, sheets or even hollow balls. Some filamentous colonies show the ability to differentiate into several different cell types, such as vegetative cells, the normal, photosynthetic cells that are formed under favorable growing conditions; akinetes, the climate-resistant spores that may form when environmental conditions become harsh; and thick-walled heterocysts, which contain the enzyme nitrogenase, vital for nitrogen fixation.

Examples of Cyanobacteria that may be utilized and/or genetically modified according to the methods described herein include Chroococcales Cyanobacteria from the genera Arthrospira, Aphanocapsa, Aphanothece, Chamaesiphon, Chroococcus, Chroogloeocystis, Coelosphaerium, Crocosphaera, Cyanobacterium, Cyanobium, Cyanodictyon, Cyanosarcina, Cyanothece, Dactylococcopsis, Gloecapsa, Gloeothece, Merismopedia, Microcystis, Radiocystis, Rhabdoderma, Snowella, Synychococcus, Synechocystis, Thermosenechococcus, and Woronichinia; Nostacales Cyanobacteria from the genera Anabaena, Anabaenopsis, Aphanizomenon, Aulosira, Calothrix, Coleodesmium, Cyanospira, Cylindrospermosis, Cylindrospermum, Fremyella, Gleotrichia, Microchaete, Nodularia, Nostoc, Rexia, Richelia, Scytonema, Sprirestis, and Toypothrix; Oscillatoriales Cyanobacteria from the genera Arthrospira, Geitlerinema, Halomicronema, Halospirulina, Katagnymene, Leptolyngbya, Limnothrix, Lyngbya, Microcoleus, Oscillatoria, Phormidium, Planktothricoides, Planktothrix, Plectonema, Pseudoanabaena/Limnothrix, Schizothrix, Symploca, Trichodesmium, and Tychonema; Pleurocapsales Cyanobacteria from the genera Chroococcidiopsis, Dermocarpa, Dermocarpella, Myxosarcina, Pleurocapsa, Stanieria, and Xenococcus; Prochlorophytes Cyanobacteria from the genera Prochloron, Prochlorococcus, and Prochlorothrix; and Stigonematales Cyanobacteria from the genera Capsosira, Chlorogeoepsis, Fischerella, Hapalosiphon, Mastigocladopsis, Nostochopsis, Stigonema, Symphyonema, Symphonemopsis, Umezakia, and Westiellopsis. In particular embodiments, the Cyanobacteria is from the genus Synechococcus, including Synechococcus bigranulatus, Synechococcus elongatus, Synechococcus leopoliensis, Synechococcus lividus, Synechococcus nidulans, and Synechococcus rubescens. Cyanobacteria Thermosynechococcus, and Gloeobacter can also be used.

More particular embodiments include or utilize Anabaena sp. strain PCC 7120, Synechocystis sp. strain PCC 6803, Nostoc muscorum, Nostoc ellipsosporum, or Nostoc sp. strain PCC 7120. In particular embodiments, the Cyanobacteria is Synechococcus elongatus sp. strain PCC 7942. Additional examples of Cyanobacteria that may utilized include Synechococcus sp. strains WH7803, WH8102, WH8103 (typically genetically modified by conjugation), Baeocyte-forming Chroococcidiopsis spp. (typically modified by conjugation/electroporation), non-heterocyst-forming filamentous strains Planktothrix sp., Plectonema boryanum M101 (typically modified by electroporation), Heterocyst-forming Anabaena sp. ATCC 29413 (typically modified by conjugation), Tolypothrix sp. strain PCC 7601 (typically modified by conjugation/electroporation) and Nostoc punctiforme strain ATCC 29133 (typically modified by conjugation/electroporation).

In particular embodiments, the Cyanobacteria may be, e.g., a marine form of Cyanobacteria or a fresh water form of Cyanobacteria. Examples of marine forms of Cyanobacteria include Synechococcus WH8102, Synechococcus RCC307, Synechococcus NKBG 15041c, and Trichodesmium. Examples of fresh water forms of Cyanobacteria include S. elongatus PCC 7942, Synechocystis PCC6803, Plectonema boryanum, Cyanobacterium aponinum, and Anabaena sp.

In particular embodiments, a genetically modified Cyanobacteria may be capable of growing in brackish or salt water. When using a fresh water form of Cyanobacteria, the overall net cost of their use will depend on both the nutrients required to grow the culture and the price for freshwater. One can foresee freshwater being a limited resource in the future, and in that case it would be more cost effective to find an alternative to freshwater. Two such alternatives include: (1) the use of waste water from treatment plants; and (2) the use of salt or brackish water.

Salt water in the oceans can range in salinity between 3.1% and 3.8%, the average being 3.5%, and this is mostly, but not entirely, made up of sodium chloride (NaCl) ions. Brackish water, on the other hand, has more salinity than freshwater, but not as much as seawater. Brackish water contains between 0.5% and 3% salinity, and thus includes a large range of salinity regimes and is therefore not precisely defined. Waste water is any water that has undergone human influence. It includes liquid waste released from domestic and commercial properties, industry, and/or agriculture and can encompass a wide range of possible contaminants at varying concentrations.

There is a broad distribution of Cyanobacteria in the oceans, with Synechococcus filling just one niche. Specifically, Synechococcus sp. PCC 7002 (formerly known as Agmenellum quadruplicatum strain PR-6) grows in brackish water, is unicellular and has an optimal growing temperature of 38° C. While this strain is well suited to grow in conditions of high salt, it will grow slowly in freshwater. In particular embodiments, the disclosure includes the use of a Cyanobacteria PCC 7942, altered in a way that allows for growth in either waste water or salt/brackish water. A Synechococcus elongatus PCC 7942 mutant resistant to sodium chloride stress has been described (Bagchi et al., Photosynth Res., 2007; 92:87-101), and a genetically modified S. elongatus PCC 7942 tolerant of growth in salt water has been described (Waditee et al., PNAS, 2002; 99:4109-4114). Salt water tolerant Cyanobacteria may also be prepared as described in the Examples of U.S. Pat. No. 8,394,614. According to the disclosure a salt water tolerant strain is capable of growing in water or media having a salinity in the range of 0.5% to 4.0% salinity, although it is not necessarily capable of growing in all salinities encompassed by this range. In particular embodiments, a salt tolerant strain is capable of growth in water or media having a salinity in the range of 1.0% to 2.0% salinity. In particular embodiments, a salt water tolerant strain is capable of growth in water or media having a salinity in the range of 2.0% to 3.0% salinity.

Up-regulating NADPH escape valves (e.g., transhydrogenases, PTOX) can be caused by, for example, an increase in the gene's copy number (or an increase in the subunits of the gene's copy number), introduction of a strong and/or inducible promoter, mechanisms to prevent degradation of encoding nucleotides or expressed proteins, or other mechanisms. Up-regulating an NADPH escape valve can also be caused by, for example, increasing its activity, increasing its binding affinity for and/or binding time of its substrates, increasing the activity of a protein or gene in a pathway that activates the NADPH escape valve, and/or decreasing the activity of a protein or gene in a pathway that inhibits the NADPH escape valve.

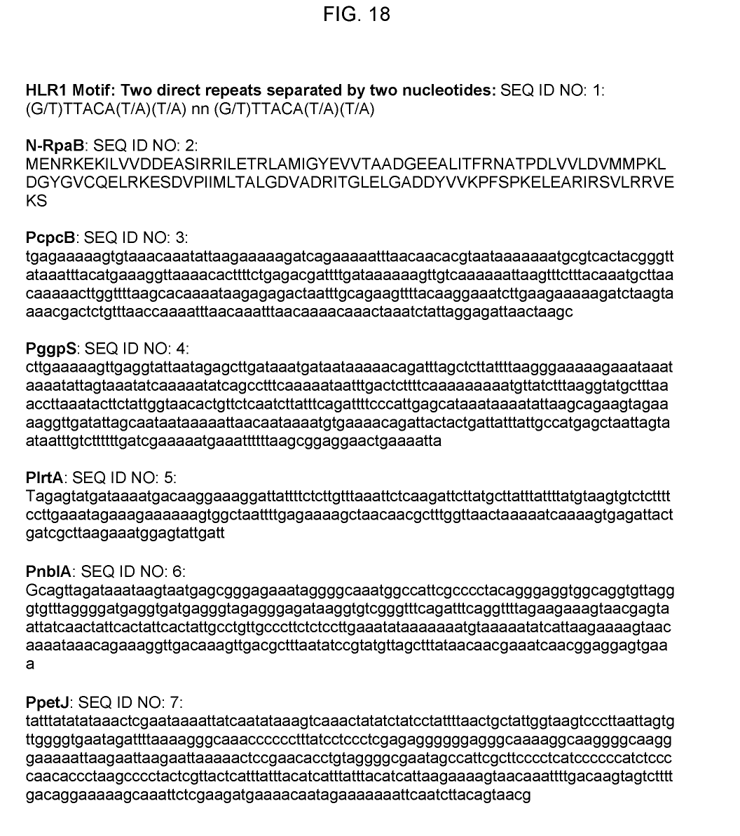

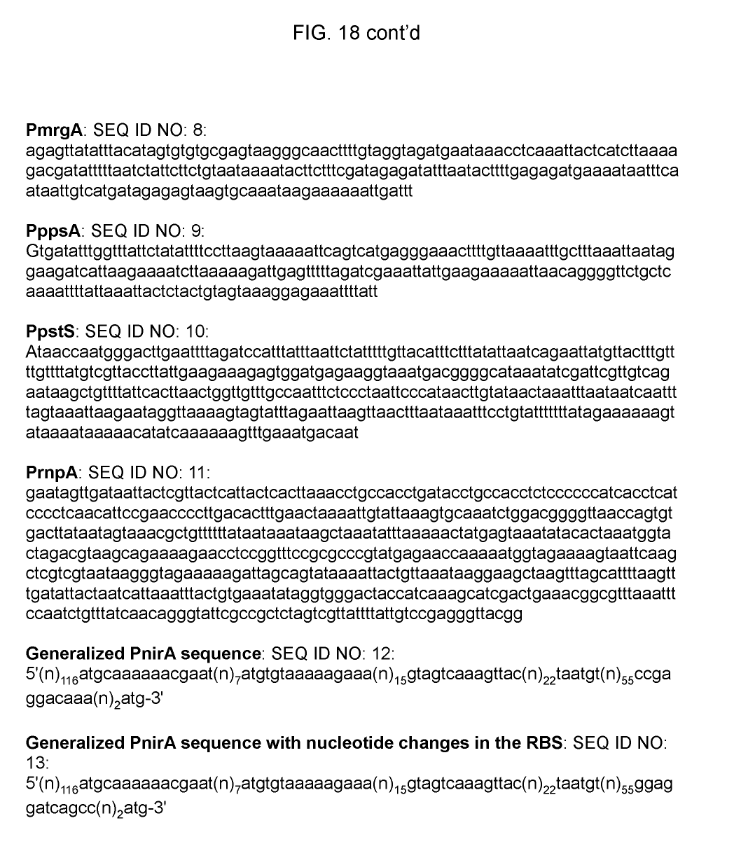

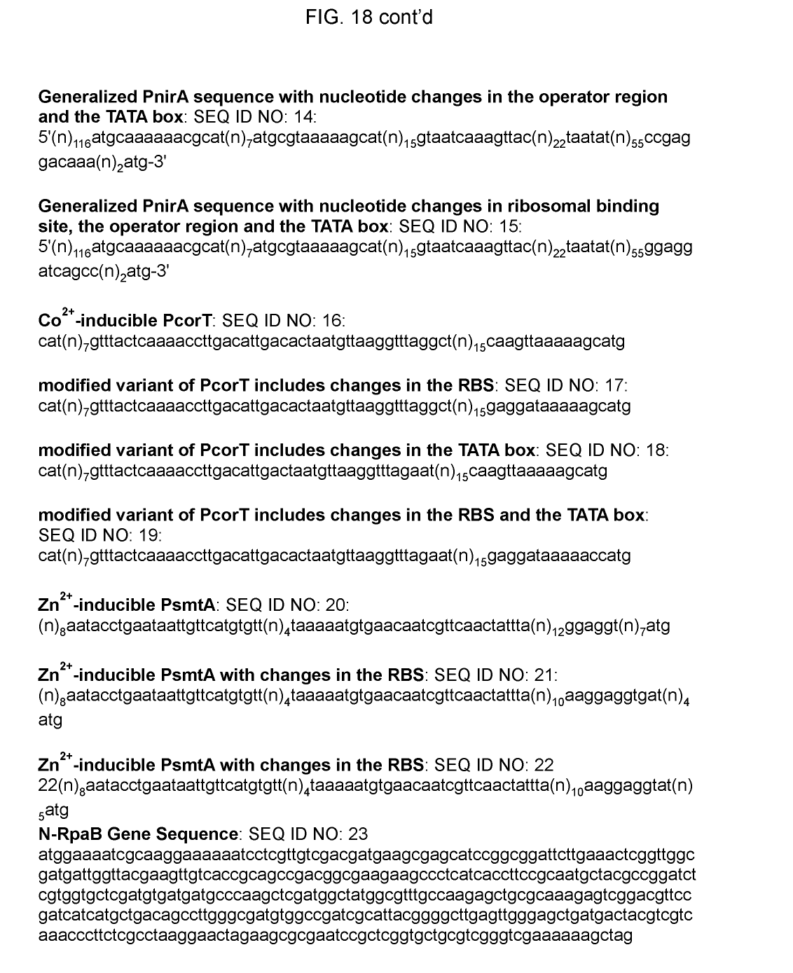

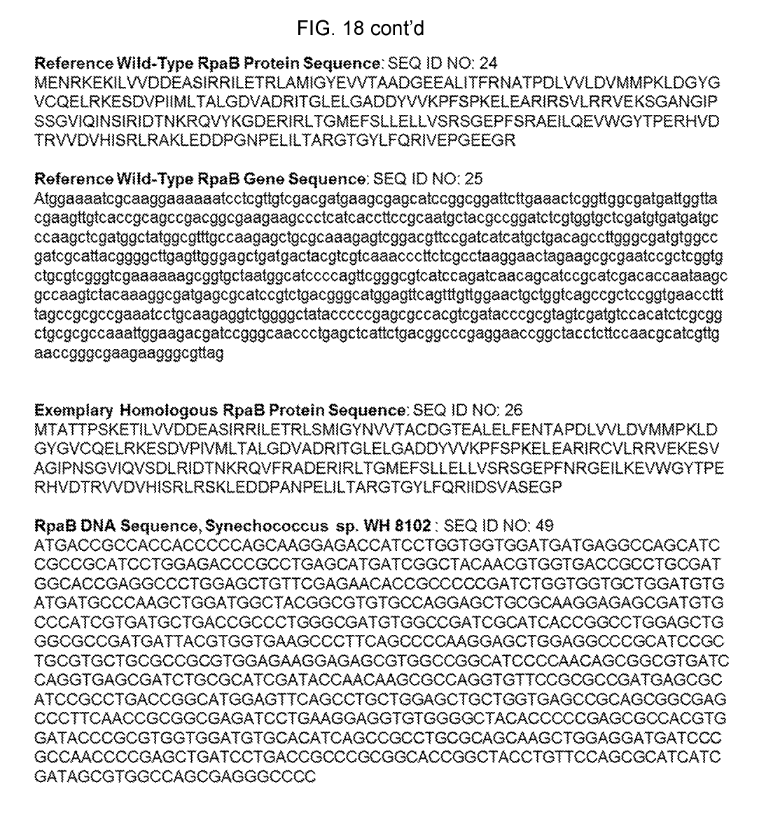

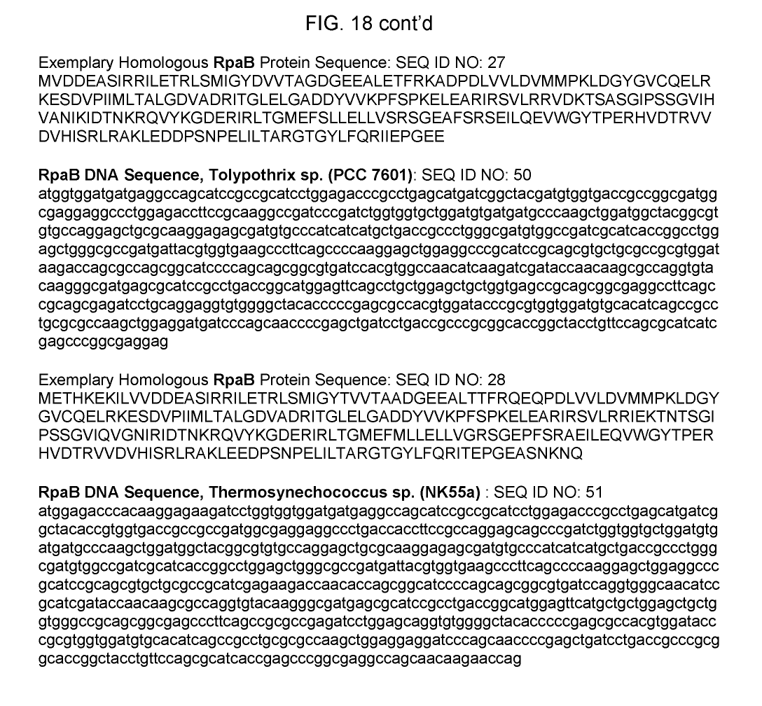

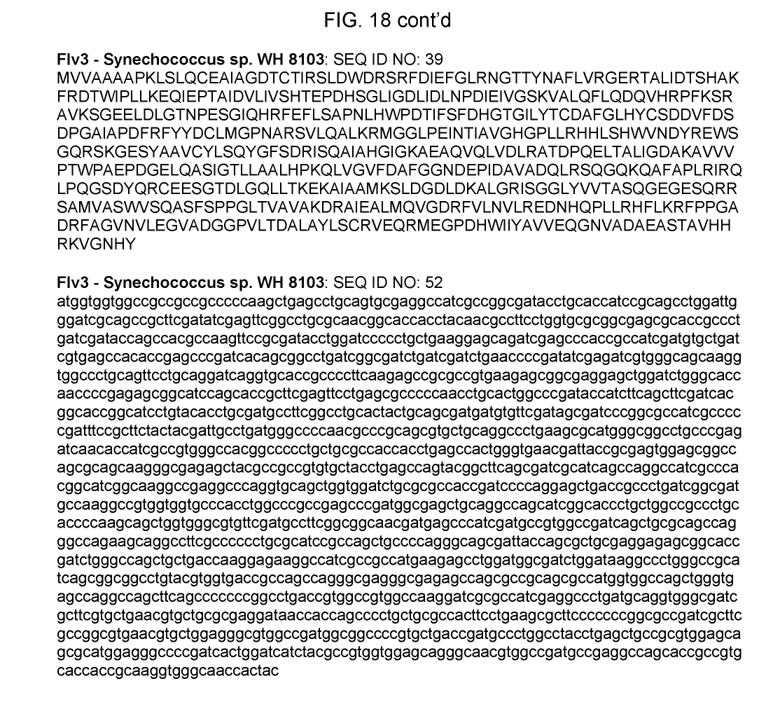

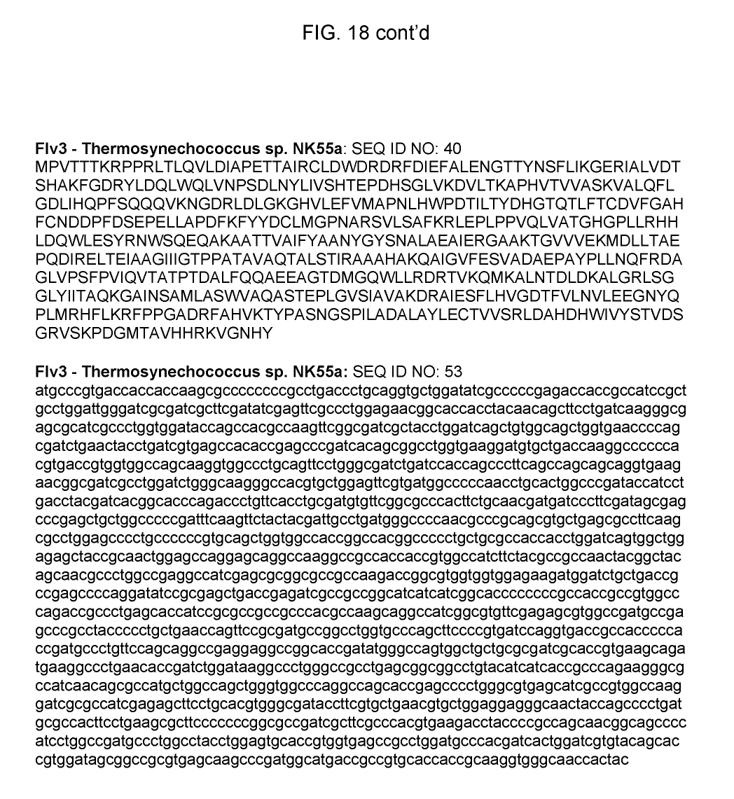

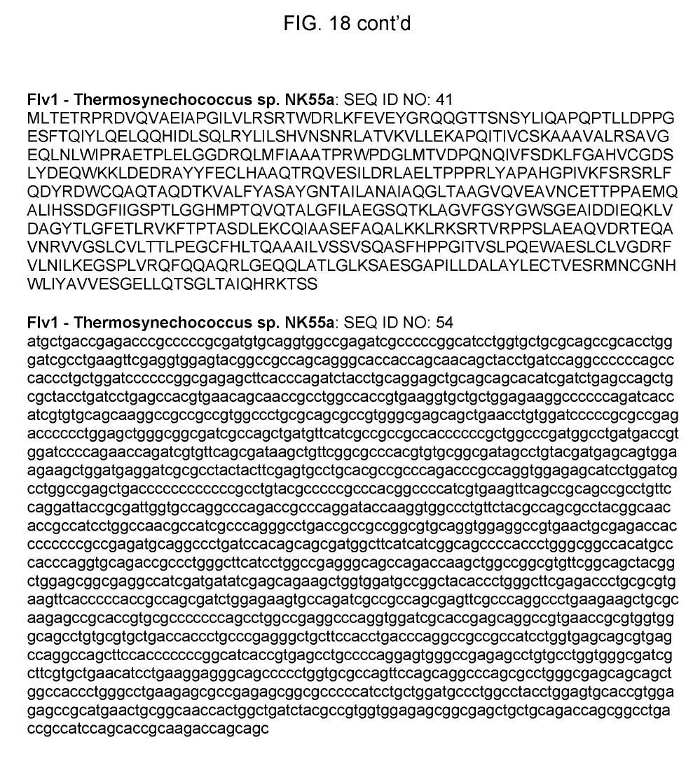

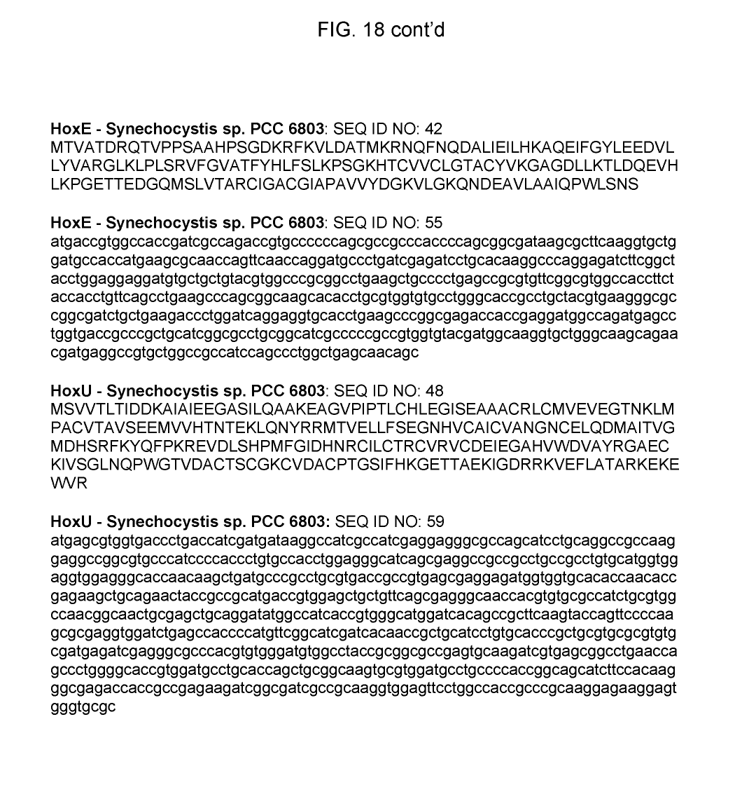

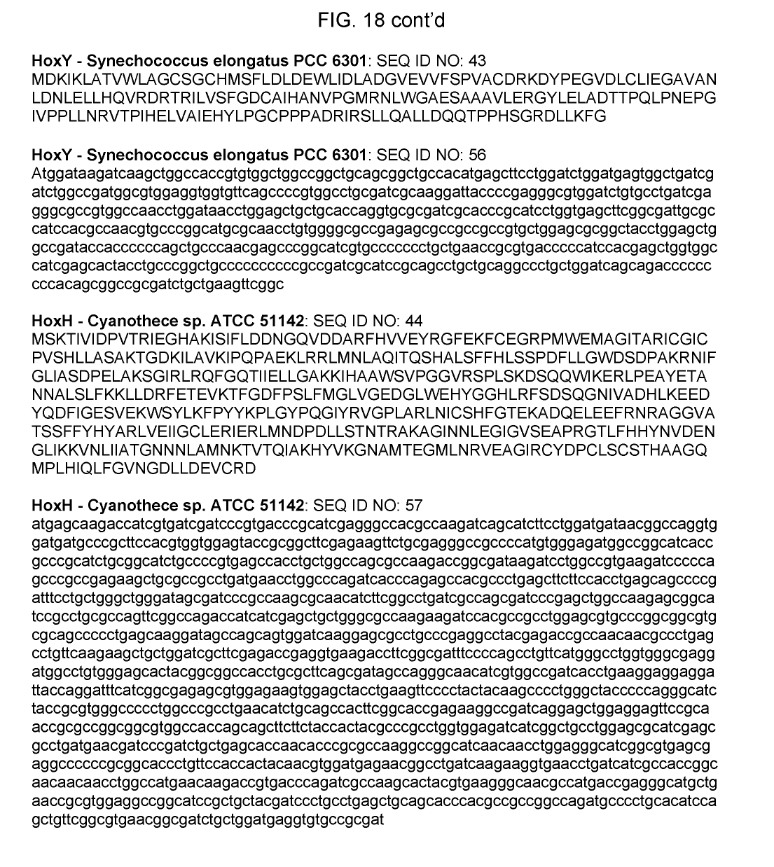

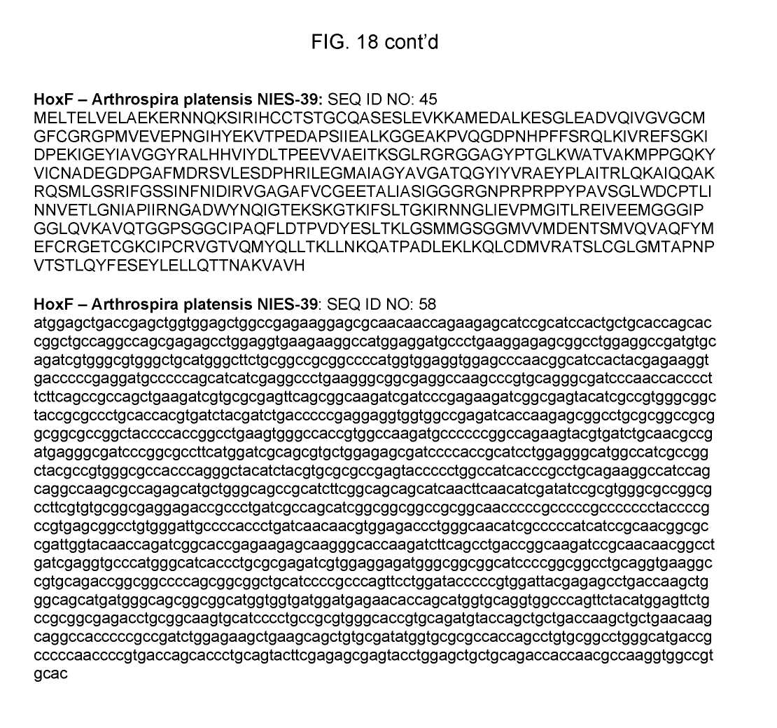

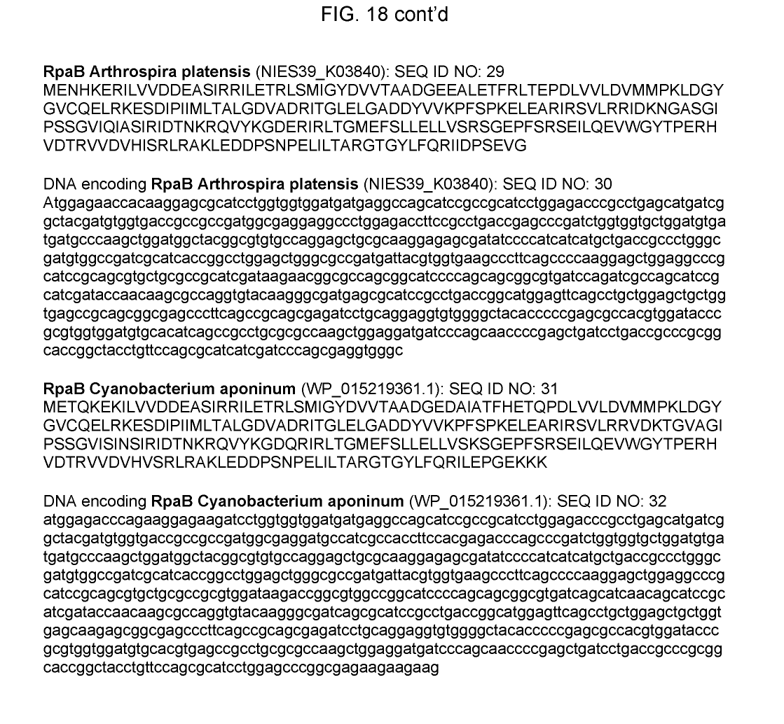

In particular embodiments, transhydrogenase is up-regulated by increasing expression of endogenous transhydrogenase genes. In particular embodiments, PTOX is up-regulated by increasing expression of endogenous PTOX genes. In particular embodiments, this type of up-regulation is achieved by inserting an exogenous promoter that leads to increased expression. In particular embodiments, transhydrogenase is upregulated by expressing an exogenous nucleotide sequence including one or more genes selected from Synpcc7942_1610, Synpcc7942_1612, and Synpcc7942_1611. In particular embodiments, PTOX is upregulated by expressing an exogenous nucleotide sequence including At4G22260 from the plant Arabidopsis thaliana. In particular embodiments, the genes are expressed as a single operon behind an inducible promoter cassette. In particular embodiments, the exogenous nucleotide sequence is inserted within neutral site 1 (NS1), neutral site 2 (NS2), neutral site 3 (NS3), neutral site 4 (NS4), and/or neutral site 5 (NS5), etc., of a cyanobacteria. Expression of (i) Flv3, (ii) Flv3 in combination with Flv1; and/or (iii) hox hydrogenase can be achieved using the approaches described for transhydrogenase and PTOX. As explained elsewhere, associated protein and gene sequences are accessible in publicly available databases. For example, reference protein sequences for Flv3 include SEQ ID NO: 39 (Synechococcus sp. WH 8103) and SEQ ID NO: 40 (Thermosynechococcus sp. NK55a). A reference protein sequences for Flv1 include SEQ ID NO: 41 (Thermosynechococcus sp. NK55a). Reference protein sequences for hox hydrogenase include SEQ ID NO: 42 (HoxE; Synechocystis sp. PCC 6803); SEQ ID NO: 48 (HoxU; Synechocystis sp. PCC 6803); SEQ ID NO: 43 (HoxY; Synechococcus elongatus PCC 6301); SEQ ID NO: 44 (HoxH; Cyanothece sp. ATC 51142); and SEQ ID NO: 45 (HoxF; Arthrospira platensis NIES-39). See FIG. 18 for exemplary gene sequences encoding hox hydrogenase, Flv3 and Flv1 proteins (SEQ ID NOs: 52-59).

Down-regulating activity of the RpaB pathway can be achieved through various mechanisms. Down-regulation of the RpaB pathway can be achieved by, for example, decreasing the presence or activity of a protein or gene in the pathway that promotes pathway activation (e.g., RpaB or its cognate kinase). Down-regulation of the RpaB pathway can also be achieved by, for example, increasing the presence or activity of a protein or gene in the pathway that inhibits pathway activation (e.g., a pathway phosphatase or a pathway decoy that dampens effective activity of other active pathway members (e.g., a wild-type RpaB sequence with the phospho-receiver domain substituted with amino acids that are not phosphorylatable (a non-conservative substitution)).

A decrease in presence or activity of a protein or gene in a pathway can be caused by, for example, reduction of a gene's copy number, insertion of a foreign set of base pairs into a gene (e.g., into a coding region), deletion of any portion of the gene (e.g., of all or part of a coding region), substitution of base pairs within the gene (e.g., into a coding region), interference with an encoded RNA transcript, the presence of antisense sequences that interfere with transcription or translation of the gene; translation of an incomplete protein; incorrect folding of a protein; expression of an unstable protein; reduced transcription of a gene; incomplete transcription of a gene, or by any other activity resulting in reduced presence, expression or activity of a protein in the pathway that promotes pathway activation.

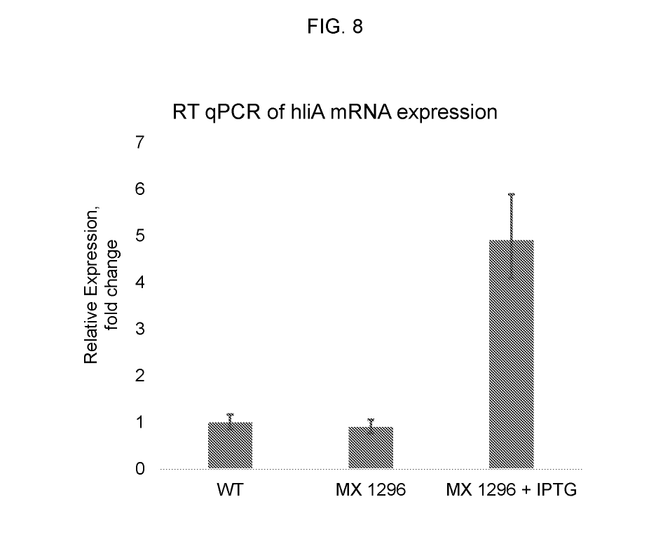

In particular embodiments, the RpaB pathway is down-regulated by expressing an RpaB decoy. Without being bound by theory, expressed RpaB decoys will compete with full length wild type RpaB for phosphorylation (e.g., on Asp56). This competition will result in a net decrease in the phosphorylation of wild type RpaB proteins, and thus up-regulation of RpaB-regulated gene expression. This approach can be referred to as a “dominant interfering” phenotype as the modified photosynthetic microorganism is expected to have a lower degree of transcriptional repression at HLR1. In other words, a constitutive “high light” (or deprivation of certain nutrient) phenotype can be created. Thus, in particular embodiments, down-regulation of the RpaB pathway can be evidenced by the up-regulation of an RpaB-regulated gene, such as hIiA (see FIG. 8) and RpoD3.

In particular embodiments, the RpaB decoy is a protein that will compete with wild-type RpaB for phosphorylation. The term “wild-type” can be used interchangeably with “naturally occurring” and refers to a gene or gene product (e.g., transcript or protein) that has the characteristics of that gene or gene product when isolated from a naturally occurring source. A wild type gene or gene product is that which is most frequently observed in a population and is thus arbitrarily designated the “normal” or “wild-type” form of the gene or gene product.

In particular embodiments, the RpaB decoy is a protein that can be phosphorylated by one or more kinases capable of phosphorylating wild-type RpaB. In particular embodiments, the RpaB decoy is a protein that can be phosphorylated by NbIS. In particular embodiments, the RpaB decoy includes a wild-type RpaB phospho-receiver domain. In particular embodiments, the RpaB decoy includes a wild-type RpaB phospho-receiver domain and 1, 2, 3, 4, or 5 wild-type amino acid residues flanking this position. In these embodiments, the phospho-receiver domain can be Asp56. In particular embodiments, the RpaB decoy includes a wild-type RpaB phospho-receiver domain and does not include a wild-type DNA binding domain. In particular embodiments, the RpaB decoy includes an N-terminal fragment of the wild-type RpaB, including the wild-type phospho-receiver domain but does not include a DNA binding domain. In particular embodiments, the wild-type phospho-receiver domain includes Asp56. As indicated below, this Asp can be replaced with phospho-receiver domain conservative substitutions such as Glu, Ser and Thr.

In particular embodiments, the RpaB decoy lacks the RpaB DNA binding domain. In particular embodiments, the RpaB decoy is an N-terminal fragment of the wild type RpaB protein that maintains Asp56. In particular embodiments, the RpaB decoy is N-RpaB (SEQ ID NO: 2).

SEQ ID NO: 24 provides a reference wild-type RpaB protein sequence and SEQ ID NO: 25 provides a reference wild-type RpaB gene sequence. These reference sequences are derived from Synpcc7942_1453. SEQ ID NO: 29 provides a reference wild-type RpaB protein sequence derived from Arthrospira platensis (NIES39_K03840) and SEQ ID NO: 30 provides a gene sequence encoding SEQ ID NO: 29 (FIG. 18). SEQ ID NO: 31 provides a reference wild-type RpaB protein sequence derived from Cyanobacterium aponinum (WP_015219361.1) and SEQ ID NO: 32 provides a gene sequence encoding SEQ ID NO: 31 (FIG. 18). Additional homologous protein and gene sequences can also serve as reference wild-type RpaB protein sequences for the purposes of this disclosure. Exemplary homologous reference RpaB protein sequences include SEQ ID NOs: 26, 27 and 28 derived from Synechococcus sp. (WH 8102), Tolypothrix sp. (PCC 7601), and Thermosynechococcus sp. (NK55a) respectively. SEQ ID NOs: 49, 50 and 51 are exemplary gene sequences that encode SEQ ID NOs: 26, 27 and 28, respectively. Based on the teachings of this disclosure, one of ordinary skill in the art can determine additional homologous sequences, relevant phospho-receiver domains, DNA binding domains, and encoding nucleotide sequences to generate functioning RpaB decoys.

Embodiments disclosed herein do not utilize RpaB knockouts, as complete RpaB knockout is lethal. Accordingly, embodiments disclosed herein that optionally down-regulate activity of the RpaB pathway utilize down-regulation, rather than elimination, of RpaB pathway activity.

As is understood by one of ordinary skill in the art, “up-regulation” of NADPH escape valves and “down-regulation” of gene and protein expression as well as RpaB pathway activity can be measured against a relevant control condition including relative to the expression or activity of an unmodified photosynthetic microorganism or a photosynthetic microorganism having a different modification (such as a modification un-related to up-regulation of a NADPH escape valve).

In particular embodiments, conclusions are drawn based on whether a measure is statistically significantly different or not statistically significantly different from a reference level of a relevant control. A measure is not statistically significantly different if the difference is within a level that would be expected to occur based on chance alone. In contrast, a statistically significant difference or increase is one that is greater than what would be expected to occur by chance alone. Statistical significance or lack thereof can be determined by any of various systems and methods used in the art. An example of a commonly used measure of statistical significance is the p-value. The p-value represents the probability of obtaining a given result equivalent to a particular datapoint, where the datapoint is the result of random chance alone. A result is often considered significant (not random chance) at a p-value less than or equal to 0.05.

As indicated, various mechanisms to up-regulate NADPH escape valves, and optionally down-regulate the RpaB pathway rely on inserting exogenous nucleotide sequences into the genome of the selected photosynthetic microorganism. “Exogenous” refers to a nucleotide sequence that does not naturally occur in the particular position of the genome of the wild type photosynthetic microorganism where it is inserted, but is inserted at the particular position by molecular biological techniques. Examples of exogenous nucleotide sequences include vectors, plasmids, and/or man-made nucleic acid constructs. Endogenous nucleotide sequences are sequences that are not introduced into a microorganism through molecular biological techniques.

As used herein, nucleotide sequences can include foreign sets of base pairs and genes encoding proteins (e.g., transhydrogenase, transhydrogenase subunits, PTOX, RpaB decoys, N-RpaB, interfering antisense sequences, etc.). In relation to genes, this term includes various sequence polymorphisms, mutations, and/or sequence variants. In particular embodiments, the sequence polymorphisms, mutations, and/or sequence variants do not affect the function of the encoded protein. Genes may include not only coding sequences but also non-coding regulatory regions such as promoters, enhancers, and termination regions. The term further can include all introns and other DNA sequences spliced from the mRNA transcript, along with variants resulting from alternative splice sites. Nucleic acid sequences encoding proteins can be DNA or RNA that directs the expression of protein or RNA. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein or RNA. The sequences can also include degenerate codons of the native sequence or sequences that may be introduced to provide codon preference. Thus, a gene refers to a unit of inheritance that occupies a specific locus on a chromosome and includes transcriptional and/or translational regulatory sequences and/or a coding region and/or non-translated sequences (i.e., introns, 5′ and 3′ untranslated sequences).

In particular embodiments, gene sequences can be used that are codon-optimized for expression by a particular organism. The frequency of codon usage of particular organisms (e.g., Synechococcus sp WH 8102), can be publicly available and used to design DNA sequences optimized for expression by the organism of interest. For example, SEQ ID NOs: 49-59 (FIG. 18) are DNA sequences encoding proteins disclosed herein, and are codon optimized for expression by Synechococcus.

A coding sequence is any nucleotide sequence that contributes to the code for the protein product of a gene. A non-coding sequence thus refers to any nucleic acid sequence that does not contribute to the code for the protein product of a gene.

In addition to particular sequences provided, gene sequences to encode for and/or interfere with proteins described herein, as well as associated RNA are available in publicly available databases and publications.

A “vector” is a nucleotide molecule, (e.g., a DNA molecule) derived, for example, from a plasmid, bacteriophage, yeast or virus, into which a nucleotide sequence (e.g., a gene) can be inserted or cloned. A vector preferably includes one or more unique restriction sites and can be capable of autonomous replication in a photosynthetic microorganism. Autonomously replicating vectors include vectors that exist as extra-chromosomal entities, the replication of which is independent of chromosomal replication, e.g., a linear or closed circular plasmid, an extra-chromosomal element, a mini-chromosome, or an artificial chromosome. Vectors can also be integrable with the genome of the photosynthetic microorganism. This type of vector is replicated together with the chromosome(s) into which it has been integrated. Such a vector may include specific sequences that allow recombination into a particular, desired site of the host chromosome. Vectors used within the current disclosure can include any mechanism for assuring self-replication. A vector can include a single vector (or plasmid), two or more vectors, three or more vectors, etc. which together contain the total DNA required for expression of a nucleotide sequence of interest to be expressed in the photosynthetic microorganism.

As indicated, coding sequences to be expressed are operably linked to a promoter, that is they are placed under the regulatory control of a promoter, which then controls the transcription and optionally the translation of the coding sequence. In the construction of heterologous promoter/structural coding sequence combinations, it is generally preferred to position the promoter at a distance from the coding sequence transcription start site that is approximately the same as the distance between that a promoter and the coding sequence it controls in its natural setting. As is known in the art, some variation in this distance can be accommodated without loss of function. Similarly, the preferred positioning of a regulatory sequence element with respect to a coding sequence to be placed under its control is defined by the positioning of the element in its natural setting; i.e., the genes from which it is derived.

“Constitutive promoters” are typically active, i.e., promote transcription, under most conditions. “Inducible promoters” are typically active only under certain conditions, such as in the presence of a given molecule factor (e.g., IPTG) or a given environmental condition. In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt induced), a heavy metal, or an antibiotic.

In particular embodiments, the promoter controlling the transcription of the coding sequence of interest can be a Cyanobacterial promoter. The promoter can be endogenous to the modified photosynthetic microorganism or can be a promoter, which was modified in order to increase its efficiency. The promoter can also be a heterologous promoter from a different photosynthetic microorganism species, such as a different Cyanobacterial or bacterial species.

In particular embodiments, the coding sequence of interest is placed under the transcriptional control of promoters (P) selected from: PaztA (e.g., from Anabaena (Nostoc) sp. strain PCC 7120); Pc1pB1; PcorT (e.g., from Synechocystis sp. PCC6803); PcrhC; PcpcB, (e.g., from Cyanobacteria ABICyano1 (SEQ ID NO: 3)); PcpcBA (e.g., from Synechocystis PCC6803); PggpS (e.g., from Cyanobacteria ABICyano1: (SEQ ID NO: 4)); PhliB; PhspA; PhtpG; PisiA; PisiB; PIrtA (e.g., from Cyanobacteria ABICyano1; SEQ ID NO: 5)); PnarB; PnblA (e.g., from Cyanobacteria ABICyano1; (SEQ ID NO: 6)); PnirA; PntcA; PpetE; PpetJ (e.g., from Cyanobacteria ABICyano1; (SEQ ID NO: 7)); PpsbA2; PpsbD; PmrgA (e.g., from Cyanobacteria ABICyano1; (SEQ ID NO: 8)); PnblA (e.g., from Nostoc sp. PCC7120); PnirA (e.g., from Cyanobacteria ABICyano1); PnrsB (e.g., from Synechocystis sp. PCC6803); PnrtA; PntcA; PppsA (e.g., from Cyanobacteria ABICyano1 (SEQ ID NO: 9)); PpsaA; PpsbD; PpstS (e.g., from Cyanobacteria ABICyano1 (SEQ ID NO: 10); PrbcL (e.g., from Synechocystis sp. PCC6803); PrbcLS; PrnpA (e.g., from Cyanobacteria ABICyano1 (SEQ ID NO: 11); PrpoA; PrpsL; PsbA2 (e.g., from Synechocystis PCC6803); PsigB; PsmtA (e.g., from Synechococcus sp. PCC 7002 and Synechococcus PCC 7942); and PziaA (e.g., from Synechocystis sp. PCC6803). Homologous promoters from other species (e.g., Synechococcus elongatus, Arthrospira maxima, Arthrospira platensis, and Cyanobacterium aponinum) as appropriate can also be used.

PhspA, Pc1pB1, and PhliB can be induced by heat shock (e.g., raising the growth temperature of the photosynthetic microorganism culture (the culture) from 300° C. to 400° C.), cold shock (e.g., reducing the growth temperature of the culture from 300° C. to 20° C.), oxidative stress (e.g., by adding oxidants such as hydrogen peroxide to the culture), or osmotic stress (e.g., by increasing the salinity of the culture). PsigB can be induced by stationary growth, heat shock, and osmotic stress. PntcA and PnblA can be induced by decreasing the concentration of nitrogen in the growth medium and PpsaA and PpsbA2 can be induced by low light or high light conditions. PhtpG can be induced by osmotic stress and heat shock. PcrhC can be induced by cold shock. An increase in copper concentration can be used to induce PpetE, whereas PpetJ is induced by decreasing copper concentration. PaztA, PsmtA, and PziaA can be induced by adding Zn²⁺. PnrsB can be induced by adding Ni²⁺. PcorT can be induced by adding cobalt. Additional details of these promoters can be found, for example, in PCT/EP2009/060526.

Useful constitutive or inducible promoters are also described in, for example: Samartzidou et al., Plant Physiol., 1998; 117:225-234; Duran et al., J. of Biol. Chem., 2004; 279:7229-7233; Singh et al., Arch Microbiol., 2006; 186:273-286; Imamura et al., FEBS Lett 2003; 554:357-362; Imamura et al., J. Biol. Chem., 2006; 281:2668-2675; Agrawal et al., Biochem. Biophys. Res. Commun., 1999; 255:47-53; Mohamed et al., Plant Mol. Biol., 1989; 13:693-700; Muramatsu et al., Plant Cell Physiol., 2006; 47:878-890; Marin et al., Plant Physiol., 2004; 136:3290-3300; Marin et al., J. Bacteriol., 2002; 184:2870-2877; Qi et al., Appl. Environ. Microbiol., 2005; 71:5678-5684; Maeda et al., J. Bacteriol., 1998; 180:4080-4088; Herranen et al., Plant Cell Physiol., 2005; 46:1484-1493; Buikema et al., Proc. Natl. Acad. Sci. USA, 2001; 98:2729-2734; Mary et al., Microbiol., 2004; 150:1271-1281; He et al., J. Biol. Chem., 2001; 276:306-314; Fang et al., Curr. Microbiol., 2004; 49:192-198; and Kappell et al., Arch. Microbiol., 2007; 187:337-342.

In the case that more than one coding sequence of interest is present, then, for example, the first, second, third, etc. coding sequence can be controlled by one promoter thereby forming a transcriptional operon. Alternatively the first, second, third, etc. coding sequence can be operably linked to different first, second, third, etc. promoters, respectively. When more than one promoter is used, all can be constitutive promoters, all can be inducible promoters, or a combination of constitutive and inducible promoters can be used.

Expression control can be tightened when mutations are introduced in the TATA-box, the operator sequence and/or the ribosomal binding site (RBS) of the promoter controlling the expression of the coding sequence so that the promoter has at least 90% sequence identity to an endogenous promoter of the modified photosynthetic microorganism. Examples of these approaches are described below in relation to promoters PnirA, PcorT and PsmtA.

In particular embodiments, PnirA can have the generalized nucleotide sequence of SEQ ID NO: 12 wherein each of the nucleotides n is independently selected from: a, t, c and g and wherein the two (atg)s in the 5′-region of the promoter are the start for NtcB binding sites, gta is the start for the NtcA binding site, ccg denotes the start of the RBS, and the 3′-atg is the start codon for the first recombinant coding sequence transcriptionally controlled by this promoter.

Another generalized DNA sequence of PnirA includes nucleotide changes in the RBS leading to the generalized DNA sequence of SEQ ID NO: 13. In particular embodiments the modified PnirA can include changes in the operator region (binding site for NtcB and NtcA) and the TATA box leading to the generalized nucleotide sequence of SEQ ID NO: 14. Another variant of PnirA combines changes in the RBS, operator region and the TATA box to form SEQ ID NO: 15.

Particular embodiments provide the Co²⁺-inducible PcorT, which has the general nucleotide sequence of SEQ ID NO: 16 wherein each of the nucleotides n is independently selected from: a, t, c and g and wherein the 5′-cat is the start codon of corR (antisense orientation) and the 3′-atg is the start codon for the first recombinant coding sequence transcriptionally controlled by this promoter. A modified variant of PcorT includes changes in the RBS having SEQ ID NO: 17. Another variant of PcorT includes changes in the TATA box having the general sequence of SEQ ID NO: 18. A third modified PcorT combines the RBS and TATA box modifications into SEQ ID NO: 19.

Furthermore the Zn²⁺-inducible PsmtA from Synechococcus PCC 7002 can be used having the generalized nucleotide sequence of SEQ ID NO: 20. Changes in the RBS can lead to the following generalized nucleotide sequences of SEQ ID NO: 21 or SEQ ID NO: 22.

Again, homologous sequences from other species (e.g., Synechococcus elongatus, Arthrospira maxima, Arthrospira platensis, and Cyanobacterium aponinum) as appropriate may also be used.

As suggested, particular embodiments include codon optimization. Codons preferred by a particular photosynthetic microorganism can be selected to, for example, increase the rate of protein expression or to produce a recombinant RNA transcript having desirable properties, such as a half-life which is longer than that of a transcript generated from the naturally occurring sequence. Such nucleotide sequences are typically referred to as “codon-optimized.”

At least some of the nucleotide sequences to be expressed in modified photosynthetic microorganisms can be codon-optimized for optimal expression in a chosen Cyanobacterial strain. The underlying rationale is that the codon usage frequency of highly expressed genes is generally correlated to the host cognate tRNA abundance. (Bulmer, Nature, 1987; 325:728-730). In particular embodiments, the codon optimization is based on the Cyanobacteria ABICyano1 (as well as its close relative species) codon usage frequency (host codon bias), in order to achieve desirable heterologous gene expression (Sharp et al., 1987; Nucleic Acids Res., 15:1281-1295). In particular embodiments, codon optimization can be based on Synechococcus elongatus PCC 7942.

Codon optimization can be performed with the assistance of publicly available software, such as Gene Designer (DNA 2.0). Additional modifications to minimize unwanted restriction sites, internal Shine-Dalgarno sequences, and other sequences such as internal termination sequences and repeat sequences can also be performed. These general codon-optimization methods have been shown to result in up to 1,000 fold higher expression of heterologous genes in target organisms (Welch et al., PLoS One 4, 2009; e7002; and Welch et al., J. of the Royal Society, 2009; Interface 6 (Suppl 4):S467-S476.

In particular embodiments, a gene from Synechococcus elongatus PCC 7942 that encodes an NADPH escape valve can be placed behind an inducible promoter (e.g., IPTG) in a neutral site (e.g., NS1 or NS2) to drive expression of the NADPH escape valve.

In particular embodiments, a gene from Synechococcus elongatus PCC 7942 that encodes transhydrogenase, or a subunit thereof, can be placed behind an inducible promoter (e.g., IPTG) in a neutral site (e.g., NS1 or NS2) to drive expression of the transhydrogenase, or subunit thereof. In particular embodiments, the gene can include Synpcc7942_1610 (SEQ ID NO: 33), Synpcc7942_1612 (SEQ ID NO: 37), and/or Synpcc7942_1611 (SEQ ID NO: 35). In particular embodiments, the gene can include Synpcc7942_1610 and Synpcc7942_1612. In particular embodiments, the gene can include Synpcc7942_1610, Synpcc7942_1612, and Synpcc7942_1611. In particular embodiments, genes encoding any of SEQ ID NOs: 39-45 or other homologous sequences may be used.

In particular embodiments, a gene from Arabidopsis thaliana that encodes PTOX can be placed behind an inducible promoter (e.g., IPTG) in a neutral site (e.g., NS5) to drive expression of the PTOX. In particular embodiments, the gene can include SEQ ID NO: 46 or other effective homologous PTOX sequences.

In particular embodiments, a gene from Synechococcus elongatus PCC 7942 that encodes a RpaB decoy can be placed behind an inducible promoter in a neutral site to drive expression of the RpaB decoy. In particular embodiments, the gene can contain the first 378 base pairs of the gene Synpcc7942_1453 (full length gene is 735 base pairs), followed by a stop codon, and can be placed behind an IPTG inducible promoter in a neutral site to drive expression of N-RpaB. This gene and associated nucleotide sequence is represented by SEQ ID NO: 23. In this sequence, the wild type start codon has been modified from TTG to ATG.

In particular embodiments, a gene that has at least 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to SEQ ID NO. 33, 35 and/or 37 can be placed behind a promoter in a neutral site to drive expression of transhydrogenase or a subunit thereof.

In particular embodiments, a gene that has at least 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to SEQ ID NO. 46 can be placed behind a promoter in a neutral site to drive expression of PTOX.

In particular embodiments, a gene that has at least 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to SEQ ID NO: 23 can be placed behind a promoter in a neutral site to drive expression of N-RpaB.

In particular embodiments, a gene that encodes transhydrogenase or a subunit thereof (e.g., SEQ ID NOs: 33, 35 and/or 37) or PTOX (e.g., SEQ ID NO: 46) can be placed behind a promoter in a neutral site to drive expression of transhydrogenase or a subunit thereof or PTOX. In particular embodiments, genes that encode a protein having 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to transhydrogenase or a subunit thereof or PTOX can be placed behind a promoter in a neutral site to drive expression of transhydrogenase or a subunit thereof or PTOX. In particular embodiments, the transhydrogenase or subunit thereof includes SEQ ID NOs. 34, 36 and/or 38 and/or the PTOX includes SEQ ID NO. 47.

In particular embodiments, a gene that encodes N-RpaB (e.g., SEQ ID NO: 23) can be placed behind a promoter in a neutral site to drive expression of N-RpaB. In particular embodiments, genes that encode a protein having 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to N-RpaB can be placed behind a promoter in a neutral site to drive expression of N-RpaB.

In particular embodiments, a gene that encodes a variant of transhydrogenase or a subunit thereof, PTOX, or N-RpaB can be placed behind a promoter in a neutral site to drive expression of the variant transhydrogenase or a subunit thereof, PTOX, or N-RpaB.

Variants of NADPH escape valves, transhydrogenase, subunits thereof, PTOX, or N-RpaB include proteins having one or more amino acid additions, deletions, stop positions, or substitutions, as compared to NADPH escape valves, transhydrogenase, subunits thereof, PTOX, or N-RpaB sequences disclosed herein. Variants of NADPH escape valves, transhydrogenase, subunits thereof, PTOX, or N-RpaB have at least 85% sequence identity; 86% sequence identity; 87% sequence identity; 88% sequence identity; 89% sequence identity; 90% sequence identity; 91% sequence identity; 92% sequence identity; 93% sequence identity; 94% sequence identity; 95% sequence identity; 96% sequence identity; 97% sequence identity; 98% sequence identity; or 99% sequence identity to NADPH escape valves, transhydrogenase, subunits thereof, PTOX, or N-RpaB and cause a statistically significant decrease in photodamage and an increase in growth, and biomass yield in high light conditions and/or low temperature growth conditions as compared to a photosynthetic microorganism that has not been modified to have up-regulated NADPH escape valve, transhydrogenase, subunits thereof, PTOX, and optionally, down-regulated RpaB pathway activity.

An amino acid substitution of NADPH escape valves, transhydrogenase, subunits thereof, PTOX, or N-RpaB can be a conservative or a non-conservative substitution. A “conservative substitution” involves a substitution found in one of the following conservative substitutions groups: Group 1: Alanine (Ala; A), Glycine (Gly; G), Serine (Ser; S), Threonine (Thr; T); Group 2: Aspartic acid (Asp; D), Glutamic acid (Glu; E); Group 3: Asparagine (Asn; N), Glutamine (Gln; Q); Group 4: Arginine (Arg; R), Lysine (Lys; K), Histidine (His; H); Group 5: Isoleucine (Ile; I), Leucine (Leu; L), Methionine (Met; M), Valine (Val; V); and Group 6: Phenylalanine (Phe; F), Tyrosine (Tyr; Y), Tryptophan (Trp; W).

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. As indicated, in particular embodiments, conservative substitutions can include substituting Asp56 with Glu, Ser, Thr or Tyr.

Non-conservative substitutions include those that affect the function of NADPH escape valves, transhydrogenase, subunits thereof, PTOX, or N-RpaB in a statistically-significant manner. Non-conservative substitutions include those in which (i) a hydrophilic residue (e.g. Ser or Thr) is substituted by a hydrophobic residue (e.g. Leu, Ile, Phe, Val, or Ala); (ii) a Cys or Pro is substituted by any other residue; (iii) a residue having an electropositive side chain (e.g. Lys, Arg, or His) is substituted by an electronegative residue (e.g. Gln or Asp); or (iv) a residue having a bulky side chain (e.g. Phe), is substituted by one not having a bulky side chain, (e.g. Gly). In particular embodiments, non-conservative substitutions can be made at Asp56 in sequences having 90% or more sequence identity with wild-type RpaB to create decoys with non-functioning phospho-receiver domains. Additional information is found in Creighton (1984) Proteins, W.H. Freeman and Company.

In particular embodiments, a gene that encodes a protein that has at least 90% sequence identity; 91% sequence identity; 92% sequence identity; 93% sequence identity; 94% sequence identity; 95% sequence identity; 96% sequence identity; 97% sequence identity; 98% sequence identity; 99% sequence identity; or 100% sequence identity to a NADPH escape valve variant, transhydrogenase variant, a transhydrogenase subunit variant, PTOX variant, or an N-RpaB variant can be placed behind a promoter in a neutral site to drive expression of an NADPH escape valve, transhydrogenase, a subunit thereof, PTOX, and/or N-RpaB.

“% sequence identity” refers to a relationship between two or more sequences, as determined by comparing the sequences. In the art, “identity” also means the degree of sequence relatedness between sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine sequence identity are designed to give the best match between the sequences tested. Methods to determine sequence identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp, CABIOS, 1989; 5:151-153 with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul et al., J. Mol. Biol., 1990; 215:403-410; DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.). Within the context of this disclosure it will be understood that where sequence analysis software is used for analysis, the results of the analysis are based on the “default values” of the program referenced. As used herein “default values” will mean any set of values or parameters which originally load with the software when first initialized.

In particular embodiments, one of ordinary skill in the art will consider the structure and function of residues within NADPH escape valves to design variants. With regard to membrane bound transhydrogenases, these proteins generally includes three domains. In particular embodiments, domains I and III are exposed to the cytosol and contain the binding sites for NAD(H) and NADP(H) respectively. Domain II spans the membrane and is not directly involved in transhydrogenation. Particular transhydrogenase variants do not include substitutions or only include conservative substitutions within the NAD(P)H binding fold. Particular transhydrogenase variants do not include substitutions or only include conservative substitutions at positions 300-500; 325-475; 340 - 432; or 348, 350, 390, 392 and 424.

With regard to PTOX, these proteins contain one conserved domain. This domain, which is common in the ferritin-like diiron-carboxylate superfamily, contains a four helix bundle and a redox active tyrosine. Proteins with this particular domain structure are resistant to cyanide and azide, but sensitive to propyl gallate.

In particular embodiments, N-RpaB variants retain Asp at position 56. In particular embodiments, no variant positions are found at position 50, 51, 52, 53, 54, 55, 56, 57, 58, 59 or 60.

Variants incorporating stop positions can be biologically active fragments. Biologically active fragments have 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. A reference sequence refers generally to an amino acid sequence or a nucleic acid coding sequence expressing an NADPH escape valve (e.g., transhydrogenase or a subunit thereof, PTOX) as disclosed herein or a variant of a protein that reduces RpaB pathway activity as described herein. SEQ ID NOs. 34, 36 and 38 provide exemplary reference sequences for transhydrogenase subunits and collectively provide a reference sequence for transhydrogenase. SEQ ID NOs. 39-45 provide reference sequences for other NADPH escape valves (e.g., Flv3, etc. and associated subunits). SEQ ID NO. 47 provides an exemplary reference sequence for PTOX. SEQ ID NO: 2 is an exemplary reference sequence for N-RpaB.

Insertion (e.g., transformation) of a nucleotide sequence (e.g., a vector) into a photosynthetic microorganism can be achieved using any appropriate method including, for example, natural transformation (e.g., natural DNA uptake; see, e.g., Chung et al., FEMS Microbiol. Lett., 1998; 164: 353-361; Frigaard et al., Methods Mol. Biol., 2004; 274:325-40; Zang et al., J. Microbiol., 2007; 45:241-245); conjugation (e.g., bi- or tri-parental mating), transduction, glass bead transformation (see, e.g., Kindle et al., J. Cell Biol., 1989; 109:2589-601; Feng et al., Mol. Biol. Rep., 2009; 36:1433-9; U.S. Pat. No. 5,661,017), silicon carbide whisker transformation (see, e.g., Dunahay et al., Methods Mol. Biol., 1997; 62: 503-9), biolistics (see, e.g., Dawson et al., Curr. Microbiol., 1997; 35: 356-62; Hallmann et al., Proc. Natl. Acad. USA, 1997; 94:7469-7474; Doestch et al., Curr. Genet., 2001; 39:49-60; Jakobiak et al., Protist, 2004; 155:381-93; Ramesh et al., Methods Mol. Biol., 2004; 274: 355-307; Tan et al., J. Microbiol., 2005; 43:361-365; Steinbrenner et al., Appl Environ. Microbiol., 2006; 72:7477-7484; Kroth, Methods Mol. Biol., 2007; 390:257-267; U.S. Pat. No. 5,661,017); electroporation (see, e.g., Kjaerulff et al., Photosynth. Res., 1994; 41:277-283; Iwai et al., Plant Cell Physiol., 2004; 45:171-5; Ravindran et al., J. Microbiol. Methods, 2006; 66:174-6; Sun et al., Gene, 2006; 377: 140-149; Wang et al., Appl. Microbiol. Biotechnol., 2007; 76:651-657; Chaurasia et al., J. Microbiol. Methods, 2008; 73:133-141; Ludwig et al., Appl. Microbiol. Biotechnol., 2008; 78:729-35), laser-mediated transformation, or incubation with DNA in the presence of or after pre-treatment with any of poly(amidoamine) dendrimers (see, e.g., Pasupathy et al., J. Biotechnol., 2008; 3:1078-82), polyethylene glycol (see, e.g., Ohnuma et al., Plant Cell Physiol., 2008; 49:117-120), cationic lipids (see, e.g., Muradawa et al., J. Biosci. Bioeng., 2008; 105: 77-80), dextran, calcium phosphate, or calcium chloride (see, e.g., Mendez-Alvarez et al., J. Bacteriol., 1994; 176:7395-7397), optionally after treatment of the cells with cell wall-degrading enzymes (see, e.g., Perrone et al., Mol. Biol. Cell, 1998; 9:3351-3365).

In addition, the vector can be modified to allow for integration into a chromosome by adding an appropriate DNA sequence homologous to the target region of the photosynthetic microorganism genome, or through in vivo transposition by introducing the mosaic ends (ME) to the vector. Once a plasmid is established in a photosynthetic microorganism, it can be present, for example, at a range of from 1 to many copies per cell.

Insertion methods described above can be used for introducing nucleotide sequences (e.g., vectors) into Cyanobacterial cells harboring an extracellular polymer layer (EPS). Non-limiting examples for Cyanobacteria with an EPS include several Nostoc and Anabaena strains, such as Nostoc commune, and Anabanena cylindrica and several Cyanothece sp. strains, such as Cyanothece PCC9224, Cyanothece CA 3, Cyanothece CE 4, Cyanothece ET5, Cyanothece ET 2, and Cyanospira capsulata ATCC 43193. Further examples of Cyanobacteria with an EPS include Aphanocapsa, Cyanobacterium, Anacystis, Chroococcus, Gloeothece, Microcystis, Synechocystis, Lyngbya, Microcoleus, Oscillatoria, Phormidium, Arthrospira, Anabaena, Cyanospira, Nostoc, Scytonema, Tolypothrix, Chlorogloeopsis, Fischerella, and Mastigocladus (see for example: De Philippis et al., J. of Applied Phycology, 2001; 13:293-299; De Philippis et al., FEMS Microbiol. Reviews, 1998; 22:151-175).

In Cyanobacteria, restriction systems can create barriers to the introduction of exogenous nucleotide sequences. Restriction systems include a restriction enzyme and a specific DNA methyltransferase. Specific methylation of the restriction enzyme recognition sequence protects DNA in the photosynthetic microorganism from degradation by the corresponding restriction enzyme. Knowledge of particular restriction systems within particular bacterial cell types can allow one to protect exogenous nucleotide sequences by methylating it at particular sites to prevent degradation by the photosynthetic microorganism's restriction system restriction enzyme(s). Thus, an understanding of these restriction systems can be helpful in choosing appropriate transformation protocols for particular bacteria. Particular restriction systems for different Cyanobacterial cells can be found at rebase.neb.com.

Nucleotide sequences used herein can include selectable markers to identify modified photosynthetic microorganisms. Selectable markers can be any identifying factor, usually an antibiotic or chemical resistance gene, that is able to be selected for based upon the marker gene's effect, such as resistance to an antibiotic, resistance to a herbicide, colorimetric markers, enzymes, fluorescent markers, and the like, wherein the effect is used to track the transformation of a nucleotide sequence of interest and/or to identify a modified photosynthetic microorganism that has inherited the nucleotide sequence of interest. Examples of selectable marker genes known and used in the art include: genes providing resistance to ampicillin, gentamycin, hygromycin, kanamycin, spectinomycin, streptomycin, fluorescent proteins (e.g., from Promega Corporation, Invitrogen, Clontech, Stratagene, BD Biosciences Pharmingen, Evrogen JSC), and the like.

Modified photosynthetic microorganisms, including Cyanobacteria, can be cultured or cultivated according to techniques known in the art, such as those described in Acreman et al., J. of Industrial Microbiol. and Biotechnol., 1994; 13:193-194), in addition to photobioreactor based techniques, such as those described in Nedbal et al., Biotechnol. Bioeng., 2008; 100:902-10. One example of typical laboratory culture conditions for Cyanobacteria is growth in BG-11 medium (ATCC Medium 616) at 30° C. in a vented culture flask with constant agitation and constant illumination at 30-100 μmole photons m⁻² sec⁻¹.

Additional media for culturing Cyanobacteria, include Aiba and Ogawa (AO) Medium, Allen and Amon Medium plus Nitrate (ATCC Medium 1142), Antia's (ANT) Medium, Aquil Medium, Ashbey's Nitrogen-free Agar, ASN-III Medium, ASP 2 Medium, ASW Medium (Artificial Seawater and derivatives), ATCC Medium 617 (BG-11 for Marine Blue-Green Algae; Modified ATCC Medium 616 [BG-11 medium]), ATCC Medium 819 (Blue-green Nitrogen-fixing Medium; ATCC Medium 616 [BG-11 medium] without NO₃), ATCC Medium 854 (ATCC Medium 616 [BG-11 medium] with Vitamin B₁₂), ATCC Medium 1047 (ATCC Medium 957 [MN marine medium] with Vitamin B₁₂), ATCC Medium 1077 (Nitrogen-fixing marine medium; ATCC Medium 957 [MN marine medium] without NO₃), ATCC Medium 1234 (BG-11 Uracil medium; ATCC Medium 616 [BG-11 medium] with uracil), Beggiatoa Medium (ATCC Medium 138), Beggiatoa Medium 2 (ATCC Medium 1193), BG-11 Medium for Blue Green Algae (ATCC Medium 616), Blue-Green (BG) Medium, Bold's Basal (BB) Medium, Castenholtz D Medium, Castenholtz D Medium Modified (Halophilic Cyanobacteria), Castenholtz DG Medium, Castenholtz DGN Medium, Castenholtz ND Medium, Chloroflexus Broth, Chloroflexus Medium (ATCC Medium 920), Chu's #10 Medium (ATCC Medium 341), Chu's #10 Medium Modified, Chu's #11 Medium Modified, DCM Medium, DYIV Medium, E27 Medium, E31 Medium and Derivatives, f/2 Medium, f/2 Medium Derivatives, Fraquil Medium (Freshwater Trace Metal-Buffered Medium), Gorham's Medium for Algae (ATCC Medium 625), h/2 Medium, Jaworski's (JM) Medium, K Medium, L1 Medium and Derivatives, MN Marine Medium (ATCC Medium 957), Plymouth Erdschreiber (PE) Medium, Prochlorococcus PC Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Arthrospira (Spirulina): ATCC Medium 1679, Spirulina (SP) Medium, van Rijn and Cohen (RC) Medium, Walsby's Medium, Yopp Medium, and Z8 Medium, among others.

In particular embodiments, modified photosynthetic microorganisms disclosed herein have increased photosynthetic capacity in high light conditions. Photosynthetic capacity is defined here as the maximum rate of electron transport through the photosynthetic electron transport chain. Photosynthetic capacity can be independently measured for different segments of the photosynthetic electron transport chain. For the segment through PSII, photosynthetic capacity can be measured by determining the rate of oxygen evolution of whole cells in the presence of para-benziquinone and potassium ferricyanide, which serve to accept electrons directly from PSII, allowing for PSII oxygen evolution to run at its maximal rate, independent of down-stream proteins in the electron transport chain. For the segment including the entire electron transport chain through PSI, increased photosynthetic capacity can be measured by determining the rate of oxygen uptake of whole cells in the presence of methyl viologen and potassium cyanide, which serve to accept electrons directly from PSI, allowing for the entire electron transport chain to run at maximal rate, independent of down-stream proteins in, e.g., carbon fixation or nitrate reduction. In this latter embodiment, the uptake rate of oxygen is a measure of the photosynthetic capacity because of the specific chemistry of the assay involving methyl viologen, which follows the following half reactions:

2H₂O→4H⁺+4e⁻+O₂

2O₂+4H⁺+4e⁻→2H₂O₂

This leads to a balanced equation below, where 1 molecule of oxygen is consumed for every molecule of O₂ that could potentially be evolved:

2H₂O+O₂→2H₂O₂

Increased photosynthetic capacity can increase total carbon fixation, production of carbon containing compounds, and growth (biomass accumulation).

### Exemplary Embodiments

1. A modified photosynthetic microorganism with decreased photodamage and/or increased ATP production, growth, and/or biomass yield in high light conditions and/or low temperature growth conditions as compared to a photosynthetic microorganism of the same species without the modification in high light conditions and/or low temperature growth conditions.
2. A modified photosynthetic microorganism of embodiment 1 wherein the modified photosynthetic microorganism is a genetically-modified photosynthetic microorganism.
3. A modified photosynthetic microorganism of embodiment 1 or 2 modified to overexpress an endogenous NADPH escape valve gene.
4. A modified photosynthetic microorganism of any of embodiments 1-3 including at least one exogenous nucleotide sequence.
5. A modified photosynthetic microorganism of embodiment 4 wherein the exogenous nucleotide sequence upregulates expression of an endogenous NADPH escape valve gene.
6. A modified photosynthetic microorganism of any of embodiments 1-5 wherein the decreased photodamage and/or increased ATP production, growth, and/or biomass yield in high light conditions results from up-regulation of at least one NADPH escape valve.
7. A modified photosynthetic microorganism of embodiment 6 wherein the at least one NADPH escape valve includes (i) Flv3, (ii) Flv3 in combination with Flv1, (iii) a hox hydrogenase, (iv) a transhydrogenase and/or (v) PTOX.
8. A modified photosynthetic microorganism of embodiment 7 wherein the up-regulated NADPH escape valve includes upregulated transhydrogenase resulting from over-expression of at least one endogenous nucleotide sequence encoding the transhydrogenase and/or at least one exogenous nucleotide sequence encoding the transhydrogenase.
9. A modified photosynthetic microorganism of embodiment 8 wherein the up-regulated transhydrogenase results from over-expression of at least one exogenous nucleotide sequence encoding transhydrogenase subunits.
10. A modified photosynthetic microorganism of embodiments 8 or 9 wherein the up-regulated transhydrogenase includes SEQ ID NO: 34, SEQ ID NO: 36, and/or SEQ ID NO: 38.
11. A modified photosynthetic microorganism of embodiments 8 or 9 wherein the up-regulated transhydrogenase includes SEQ ID NO: 34, SEQ ID NO: 36, and SEQ ID NO: 38.
12. A modified photosynthetic microorganism of embodiment 9 wherein the exogenous nucleotide sequence includes SEQ ID NO: 33, SEQ ID NO: 35, and/or SEQ ID NO: 37.
13. A modified photosynthetic microorganism of embodiment 9 wherein the exogenous nucleotide sequence includes SEQ ID NO: 33, SEQ ID NO: 35, and SEQ ID NO: 37.
14. A modified photosynthetic microorganism of embodiment 7 wherein the up-regulated NADPH escape valve includes upregulated PTOX resulting from over-expression of at least one endogenous nucleotide sequence encoding the PTOX and/or at least one exogenous nucleotide sequence encoding the PTOX.
15. A modified photosynthetic microorganism of embodiment 14 wherein the up-regulated PTOX includes SEQ ID NO: 47 and/or a PTOX from Arabidopsis thaliana.
16. A modified photosynthetic microorganism of embodiment 14 wherein the exogenous nucleotide sequence includes SEQ ID NO: 46 and/or a PTOX gene from Arabidopsis thaliana.
17. A modified photosynthetic microorganism of any of embodiments 1-16 including upregulated Flv3 resulting from over-expression of at least one endogenous nucleotide sequence encoding the Flv3 and/or at least one exogenous nucleotide sequence encoding the Flv3.
18. A modified photosynthetic microorganism of embodiment 17 wherein the up-regulated Flv3 comprises SEQ ID NO: 39 and/or SEQ ID NO: 40.
19. A modified photosynthetic microorganism of embodiment 17 or 18 wherein the exogenous nucleotide sequence comprises SEQ ID NO: 52 and/or SEQ ID NO: 53.
20. A modified photosynthetic microorganism of any of embodiments 1-19 including an exogenous nucleotide sequence encoding an NADPH escape valve behind an inducible or constitutive promoter.
21. A modified photosynthetic microorganism of any of embodiments 1-20 including an exogenous nucleotide sequence encoding an NADPH escape valve behind an inducible or constitutive promoter wherein the exogenous nucleotide sequence is inserted in neutral site 1 (NS1), neutral site 2 (NS2), neutral site 3 (NS3), neutral site 4 (NS4), or neutral site 5 (NS5).
22. A modified photosynthetic microorganism of any of embodiments 1-21 wherein the photosynthetic microorganism shows decreased photodamage in high light conditions.
23. A modified photosynthetic microorganism of any of embodiments 1-22 wherein the photosynthetic microorganism shows increased ATP production in high light conditions.
24. A modified photosynthetic microorganism of any of embodiments 1-23 wherein the photosynthetic microorganism shows increased growth in high light conditions and/or low temperature growth conditions.
25. A modified photosynthetic microorganism of any of embodiments 1-24 wherein the photosynthetic microorganism shows increased biomass yield in high light conditions and/or low temperature growth conditions.
26. A modified photosynthetic microorganism of any of embodiments 1-25 having decreased RpaB pathway activity.
27. A modified photosynthetic microorganism of embodiment 26 wherein the decreased RpaB pathway activity results from (i) expression of at least one exogenous nucleotide sequence and/or (ii) a wild-type nucleotide sequence deletion wherein (i) and/or (ii) decreases RpaB pathway activity as compared to a photosynthetic microorganism of the same species without the modification(s).
28. A modified photosynthetic microorganism of embodiment 27 wherein one or more exogenous nucleotide sequences (i) result in translation of an incomplete or unstable RpaB protein; (ii) result in translation of an RpaB protein that folds incorrectly; (iii) reduce transcription of the RpaB gene; (iv) result in incomplete transcription of the RpaB gene; (v) interfere with an encoded RpaB RNA transcript and/or (vi) reduce translation of RpaB.
29. A modified photosynthetic microorganism of embodiment 27 or 28 wherein one or more exogenous nucleotide sequences include a foreign set of base pairs inserted or substituted into the RpaB coding region.
30. A modified photosynthetic microorganism of any of embodiments 27-29 wherein one or more exogenous nucleotide sequences include an antisense sequence that interferes with transcription or translation of the RpaB gene.
31. A modified photosynthetic microorganism of any of embodiments 27-30 wherein one or more exogenous nucleotide sequences expresses an RpaB decoy under the control of a promoter.
32. A modified photosynthetic microorganism of any of the proceeding embodiments including at least two of the described exogenous nucleotide sequences, at least three of the described exogenous nucleotide sequences, at least four of the described exogenous nucleotide sequences, or at least five of the described exogenous nucleotide sequences.
33. A modified photosynthetic microorganism of embodiment 32 wherein the RpaB decoy is a fragment of wild type RpaB including Asp56 or a conservative substitution thereof.
34. A modified photosynthetic microorganism of embodiment 32 wherein the RpaB decoy is N-RpaB.
35. A modified photosynthetic microorganism of embodiment 32 wherein the RpaB decoy is an N-RpaB variant that maintains Asp56 or a conservative substitution thereof.
36. A modified photosynthetic microorganism of any of the proceeding embodiments wherein the modified photosynthetic microorganism is a Cyanobacteria.
37. A modified photosynthetic microorganism of any of the proceeding embodiments wherein the modified photosynthetic microorganism is a Cyanobacteria selected from Synechococcus elongatus, Arthrospira maxima, Arthrospira platensis, and Cyanobacterium aponinum.
38. A modified photosynthetic microorganism of embodiment 32 wherein the exogenous nucleotide sequence includes SEQ ID NO: 23.
39. A modified photosynthetic microorganism of any of the proceeding embodiments wherein the promoter is an inducible promoter, in particular embodiments including an IPTG inducible promoter.
40. A modified photosynthetic microorganism of any of the proceeding embodiments wherein the promoter is endogenous to the genome of the genetically-modified photosynthetic microorganism.
41. A modified photosynthetic microorganism of any of the proceeding embodiments wherein the genetically-modified photosynthetic microorganism has decreased photodamage, increased ATP production, growth, and biomass yield as compared to a wild type photosynthetic microorganism of the same species in high light conditions and/or low temperature growth conditions.
42. A method for decreasing photodamage and/or increasing ATP production, growth and/or biomass yield of a photosynthetic microorganism in high light conditions and/or low temperature growth conditions including modifying the photosynthetic microorganism to up-regulate at least one NADPH escape valve as compared to a photosynthetic microorganism of the same species without the modification in high light conditions and/or low temperature growth conditions.
43. A method of embodiment 42 wherein the modifying includes genetically modifying.
44. A method of embodiment 42 or 43 wherein the modifying includes initiating over-expression of at least one endogenous NADPH escape valve gene.
45. A method of any of embodiments 42-44 wherein the modifying includes inserting at least one exogenous nucleotide sequence into the photosynthetic microorganism.
46. A method of any of embodiments 42-45 wherein the at least one NADPH escape valve includes (i) Flv3, (ii) Flv3 in combination with Flv1, (iii) a hox hydrogenase, (iv) a transhydrogenase, and/or (v) PTOX.
47. A method of embodiment 46 wherein the at least one up-regulated NADPH escape valve includes a transhydrogenase.
48. A method of embodiment 47 wherein the at least one up-regulated transhydrogenase results from over-expressing at least one exogenous nucleotide sequence encoding the transhydrogenase.
49. A method of embodiment 47 wherein the up-regulated transhydrogenase results from over-expressing at least one exogenous nucleotide sequence encoding transhydrogenase subunits.
50. A method of any of embodiments 47, 48, or 49 wherein the up-regulated transhydrogenase includes SEQ ID NO: 34, SEQ ID NO: 36, and/or SEQ ID NO: 38.
51. A method of any of embodiments 47, 48, or 49 wherein the up-regulated transhydrogenase includes SEQ ID NO: 34, SEQ ID NO: 36, and SEQ ID NO: 38.
52. A method of any of embodiments 47, 48, or 49 wherein the exogenous nucleotide sequence includes SEQ ID NO: 33, SEQ ID NO: 35, and/or SEQ ID NO: 37.
53. A method of any of embodiments 47, 48, or 49 wherein the exogenous nucleotide sequence includes SEQ ID NO: 33, SEQ ID NO: 35, and SEQ ID NO: 37.
54. A method of any of embodiments 46-53 wherein the up-regulated NADPH escape valve includes upregulated PTOX resulting from over-expression of at least one endogenous nucleotide sequence encoding the PTOX and/or at least one exogenous nucleotide sequence encoding the PTOX.
55. A method of embodiment 54 wherein the up-regulated PTOX includes SEQ ID NO: 47 and/or a PTOX from Arabidopsis thaliana.
56. A method of embodiment 54 or 55 wherein the exogenous nucleotide sequence includes SEQ ID NO: 46 and/or a PTOX gene from Arabidopsis thaliana.
57. A method of any of embodiments 46-56 wherein the up-regulated NADPH escape valve includes upregulated Flv3 resulting from over-expression of at least one endogenous nucleotide sequence encoding the Flv3 and/or at least one exogenous nucleotide sequence encoding the Flv3.
58. A method of embodiment 57 wherein the up-regulated Flv3 includes SEQ ID NO: 39 and/or SEQ ID NO: 40.
59. A method of embodiment 57 or 58 wherein the exogenous nucleotide sequence includes SEQ ID NO: 52 and/or SEQ ID NO: 53.
60. A method of any of embodiments 42-59 including an exogenous nucleotide sequence encoding an NADPH escape valve behind a constitutive or inducible promoter.
61. A method of any of embodiments 42-60 including an exogenous nucleotide sequence encoding an NADPH escape valve behind an inducible promoter wherein the exogenous nucleotide sequence is inserted in neutral site 1 (NS1), neutral site 2 (NS2), neutral site 3 (NS3), neutral site 4 (NS4), or neutral site 5 (NS5).
62. A method of any embodiments 42-61 wherein the modified photosynthetic microorganism shows reduced photodamage in high light conditions.
63. A method of any embodiments 42-62 wherein the photosynthetic microorganism shows increased ATP production in high light conditions.
64. A method of any embodiments 42-63 wherein the photosynthetic microorganism shows increased growth in high light conditions and/or low temperature growth conditions.
65. A method of any embodiments 42-64 wherein the photosynthetic microorganism shows increased biomass yield in high light conditions and/or low temperature growth conditions.
66. A method of any embodiments 42-65 further comprising modifying the photosynthetic microorganism to down-regulate RpaB pathway activity within the photosynthetic microorganism as compared to a photosynthetic microorganism of the same species without the modification.
67. A method of embodiment 66 wherein the modifying includes genetically modifying the photosynthetic microorganism.
68. A method of embodiment 67 wherein the modifying includes inserting at least one exogenous nucleotide sequence into the photosynthetic microorganism or deleting an endogenous nucleotide sequence from the photosynthetic microorganism.
69. A method of embodiments 68 wherein the at least one exogenous nucleotide sequence (i) results in translation of an incomplete or unstable RpaB protein; (ii) results in translation of an RpaB protein that folds incorrectly; (iii) reduces transcription of the RpaB gene; (iv) results in incomplete transcription of the RpaB gene; (v) interferes with an encoded RpaB RNA transcript and/or (vi) reduces translation of RpaB.
70. A method of any embodiments 66-69 wherein modifying includes inserting or substituting a foreign set of base pairs into the RpaB coding region.
71. A method of any embodiments 66-70 wherein modifying includes inserting an antisense sequence that interferes with transcription or translation of the RpaB gene.
72. A method of any embodiments 66-71 wherein the modifying includes inserting an exogenous nucleotide sequence that expresses an RpaB decoy under the control of a promoter.
73. A method of any embodiments 66-72 including at least two of the described modifications, at least three of the described modifications, at least four of the described modifications, or at least five of the described modifications.
74. A method of any embodiments 66-73 wherein the modifying includes inserting an exogenous nucleotide sequence that expresses an N-terminal fragment of wild type RpaB including Asp56 or a conservative substitution thereof.
75. A method of any embodiments 66-74 wherein the modifying includes inserting an exogenous nucleotide sequence that expresses N-RpaB.
76. A method of any embodiments 66-75 wherein the modifying includes inserting an exogenous nucleotide sequence that expresses an N-RpaB variant that maintains Asp56 or a conservative substitution thereof.
77. A method of any embodiments 66-76 wherein the modifying includes modifying a Cyanobacteria.
78. A method of any embodiments 66-77 wherein the modifying includes modifying a Cyanobacteria selected from Synechococcus elongatus, Arthrospira maxima, Arthrospira platensis, and Cyanobacterium aponinum.
79. A method of any embodiments 66-78 wherein the modifying includes inserting an exogenous nucleotide sequence including SEQ ID NO: 23 into the photosynthetic microorganism.
80. A method of any embodiments 66-79 wherein the modifying includes inserting an exogenous inducible promoter (e.g., IPTG) into the photosynthetic microorganism.
81. A method of any embodiments 66-80 wherein the modifying includes inserting an exogenous promoter that is endogenous to the genome of the genetically-modified photosynthetic microorganism.

When an inserted exogenous promoter is endogenous to the genome of the genetically-modified photosynthetic microorganism, this means that an extra copy of a wild-type promoter of the species is inserted as part of a genetic construct.

The Examples below describe the optimization of the methods disclosed herein. These Examples are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXAMPLE 1

The three transhydrogenase genes that were overexpressed included Synpcc7942_1610, Synpcc7942_1611, and Synpcc7942_1612. These genes were overexpressed in a single operon behind an IPTG inducible promoter cassette (Ptrc). Homologous transhydrogenase genes to Synpcc7942_1610, Synpcc7942_1611, and Synpcc7942_1612in the genome of Synechocystis sp. PCC 6803 are called pntA and pntB.

The strain was made by transforming a plasmid, pMX1137, into control strains TGA1-30 (wild type) and MX1504 (a strain called “NrpaB” which overexpresses an N-terminal fragment of RpaB (Synpcc7942_1453).

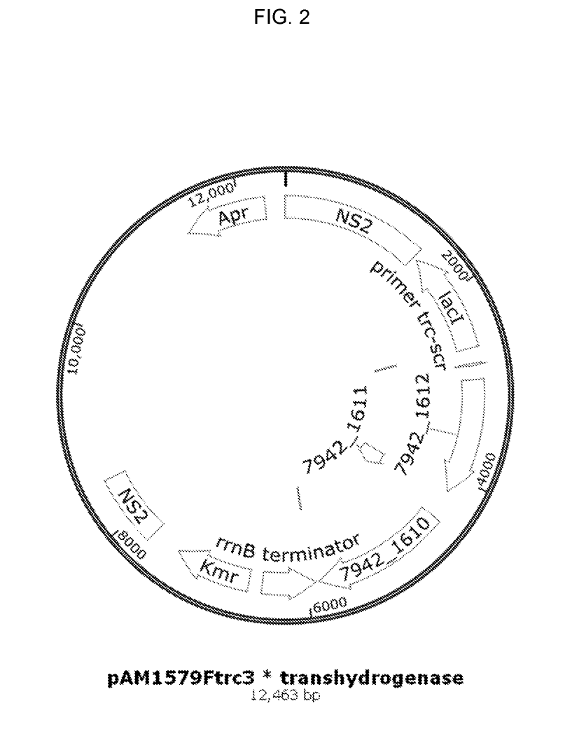

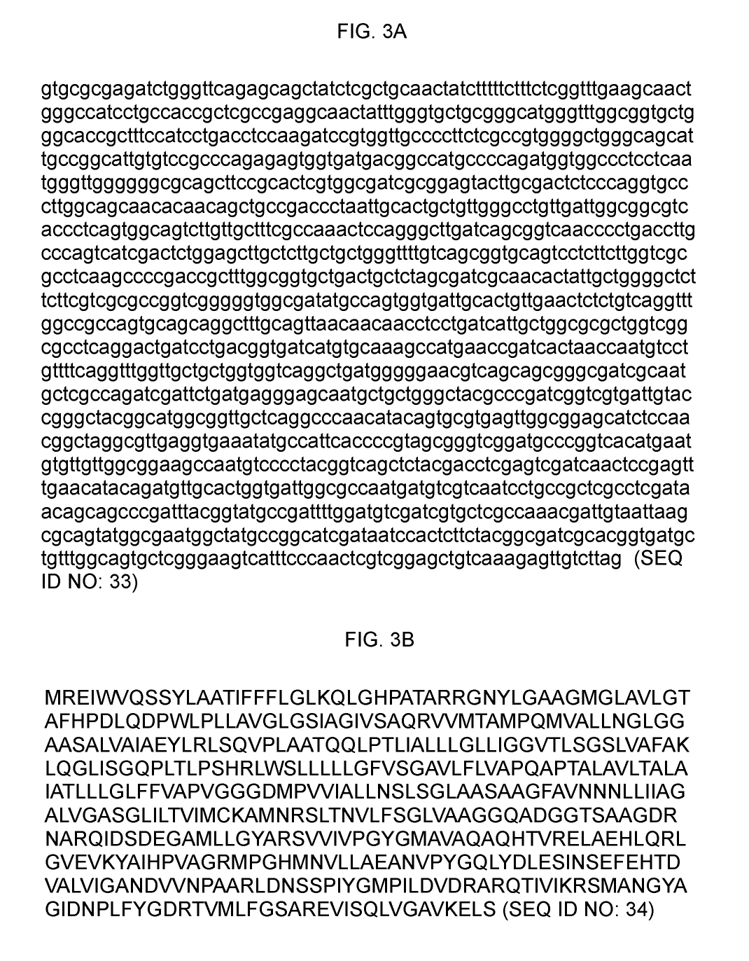

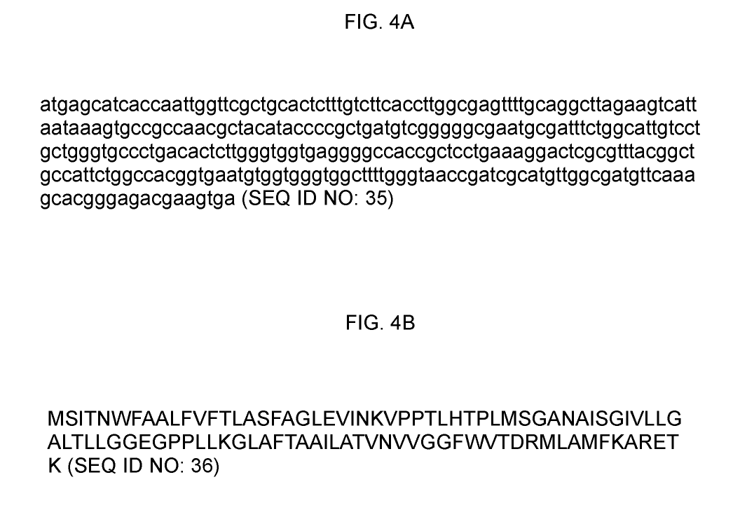

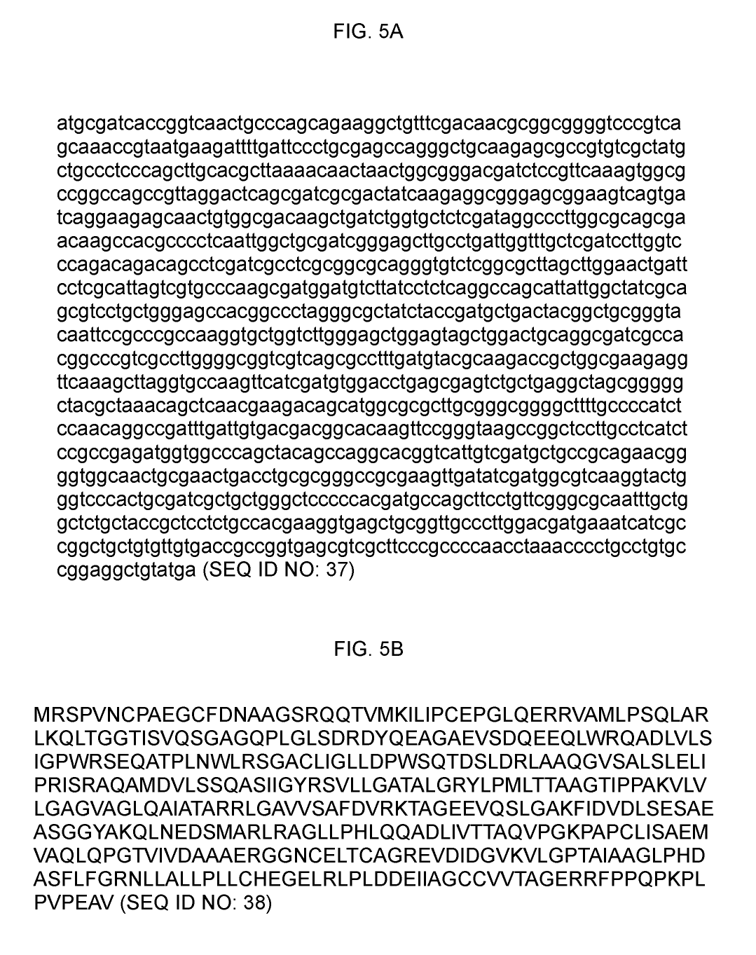

pMX1137 was designed to insert the transhydrogenase overexpression construct plus a kanamycin resistance cassette (KmR) into a region of the genome (neutral site 2 or “NS2”) by homologous recombination. The Plasmid map of pMX1137 is shown below in FIG. 2. The DNA nucleotide and Protein amino acid sequences of Synpcc7942_1610, Synpcc7942_1611, and Synpcc7942_1612 are shown in FIGS. 3A, 3B, 4A, 4B, 5A, and 5B, respectively.

Successful transformation of pMX1137 into both TGA 1-30 and MX1504 followed by gene insertion and homoplasmy resulted in strains denoted MX2134 and MX2135, respectively. Gene insertion and homoplasmy were verified by PCR.

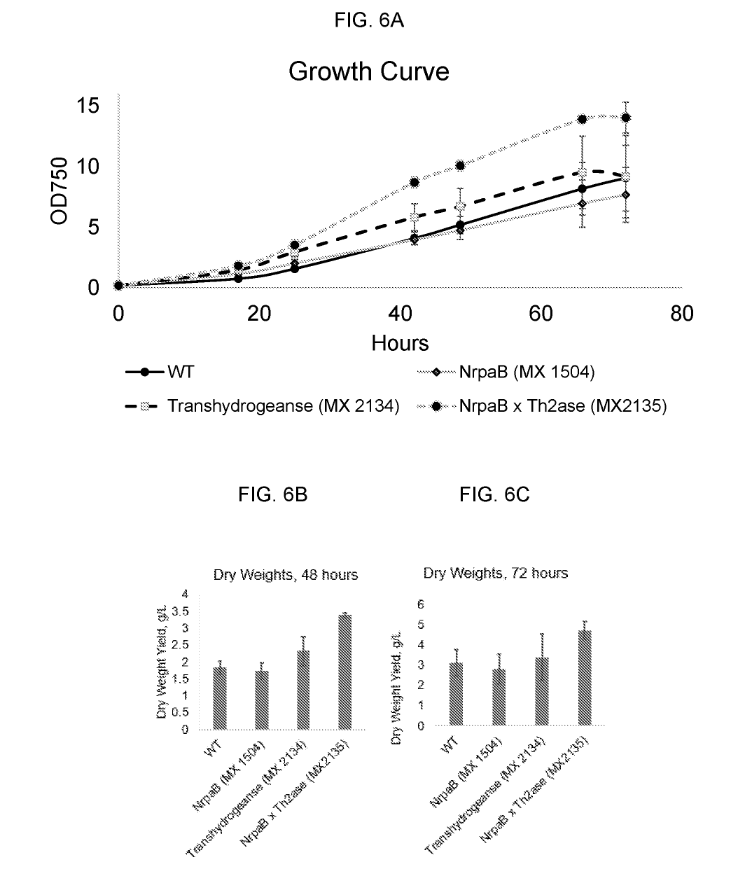

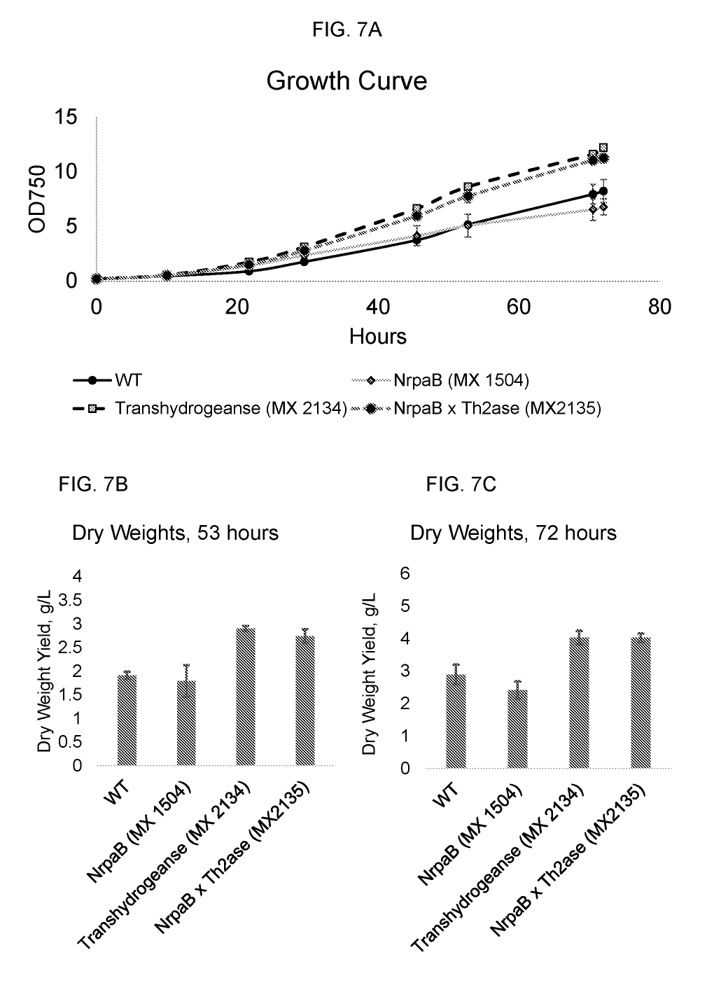

Initial growth experiments under 1000 μE*m⁻²*s⁻¹ continuous illumination flux and 1% CO₂/air atmosphere showed improvements in growth rates and yields of transhydrogenase overexpression strains MX2134 and MX2135 relative to respective controls WT (TGA 1-30) and NrpaB (MX1504). FIG. 6 shows a growth time-course of these 4 strains and dry biomass yields of these cultures after 48 and 72 hours of growth in continuous light. FIG. 7 shows a similar set of data for a repeat experiment under similar conditions. These results were unexpected because reports in the literature show that overexpression of a transhydrogenase encoding gene from E. coli, udhA, results in decreased growth rates in both S7942 and Synechocystis sp. PCC 6803 (Angermayr, et al., (2012) Applied and Environmental Microbiology 78: 7098-7106; Niederholtmeyer, et al., (2010) Applied and Environmental Microbiology 76: 3462-3466). However, udhA is a single gene-encoded enzyme that is termed “soluble” because it is thought to most present in the cytosol of bacterial cells while the transhydrogenase used in this Example is a two or three gene encoded enzyme. udhA is not homologous to Synpcc7942_1610 or Synpcc7942_1611 and shows only 21% similarity with Synpcc7942_1612 (as determined by EMBOSS needle alignment).

### EXAMPLE 2

A gene from Synechococcus elongatus PCC 7942 that contains the first 378 base pairs of the gene Synpcc7942_1453 (full length gene is 735 base pairs), followed by a stop codon, was placed behind an IPTG inducible promoter in a neutral site (NS1) to drive expression of a truncated form of RpaB. When the strain with this mutation is grown in the presence of 1 mM IPTG, it has a higher photosystem II activity and higher photosynthetic electron transport capacity than wild type. Photosystem II activity is measured by determining the rate of oxygen evolution of whole cells in the presence of para-benziquinone and potassium ferricyanide, which serve to accept electrons directly from PSII, allowing for PSII oxygen evolution to run at its maximal rate, independent of down-stream proteins in the electron transport chain. Electron transport activity is measured by determining the rate of oxygen consumption of whole cells under bright light illumination in the presence of methyl viologen and potassium cyanide. The magnitude of the measured rate of oxygen uptake is equal to the total capacity of the cell's oxygen evolution rate when electrons are passed through its entire electron transport chain via photosynthesis. Overexpression of the same 378 base pair gene fragment under other strong promoters also confers an increase in electron transport activity.

Overexpression of the N-RpaB protein fragment conferred a transcriptional response similar to one observed when cells are stressed with, e.g. high light or deprivation of certain nutrients. For example, the mRNA expression level of the gene hIiA, which is known to increase under high light, increases 5-fold relative to wild type or uninduced mutant levels, as shown in FIG. 8.

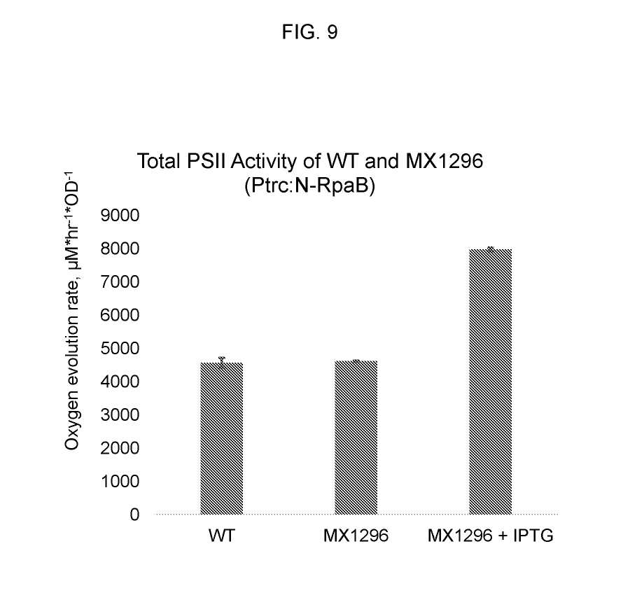

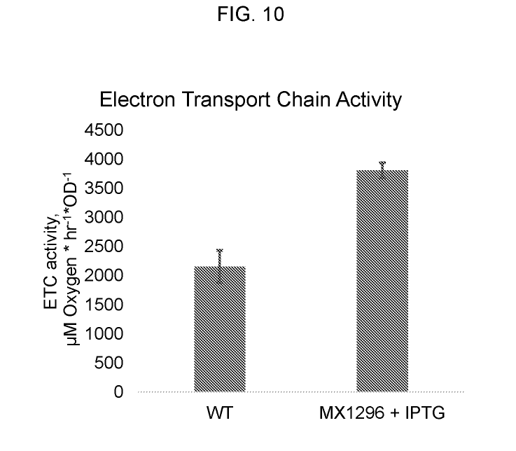

One of the physiological responses observed in mutants with that overexpress N-RpaB is an increase in total activity of PSII per cell. FIG. 9 shows the maximal oxygen evolution capacity of PSII in cells with induction of the RpaB protein fragment (denoted N-RpaB) with 1 mM IPTG. Without being bound by theory, the increase in PSII capacity allows for increased total electron transport chain activity in Synechococcus. FIG. 10 shows the maximal electron transport chain activity of cells with and without induced production of N-RpaB.

### EXAMPLE 3

PTOX Escape Valves. The gene that was overexpressed is called At4G22260. It encodes the PTOX protein from the plant Arabidopsis thaliana. It was overexpressed as a single transcript behind an IPTG inducible promoter cassette (Ptrc). The strain MX2349 was made by transforming a plasmid, pMX1317, into control strains TGA1-30 (wild type).

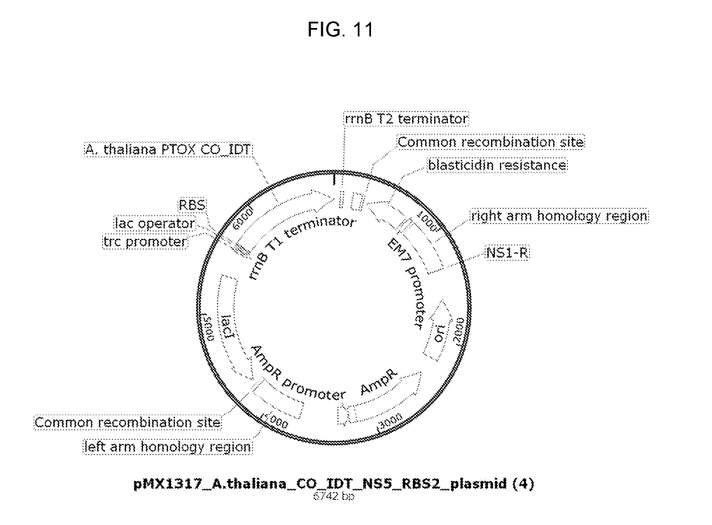

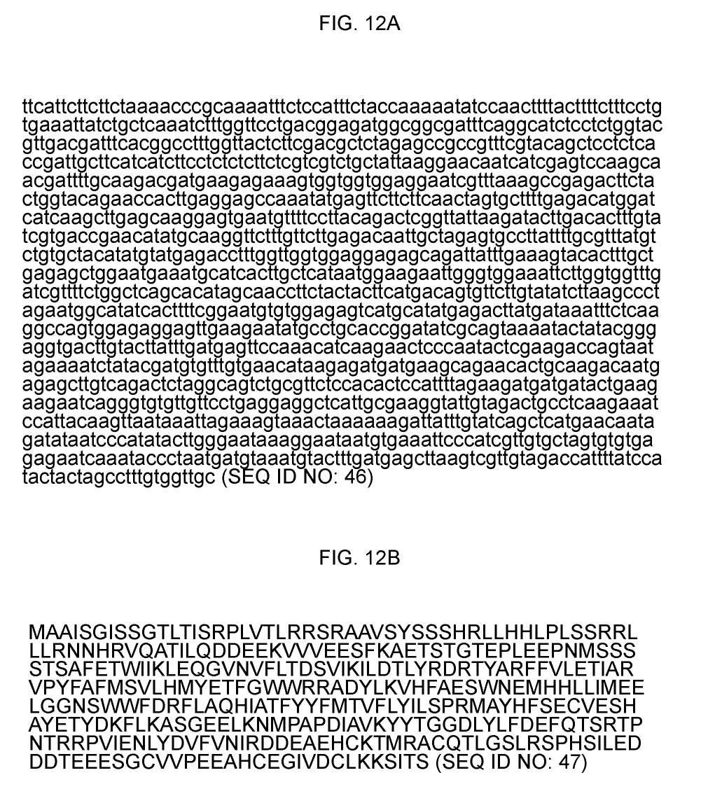

pMX1317 was designed to insert the PTOX overexpression construct plus a blastocyanin resistance cassette (BsR) into a region of the genome (neutral site 5 or “NS5”) by homologous recombination. The Plasmid map of pMX1317 is in FIG. 11. The DNA nucleotide and Protein amino acid sequences of At4G22260 are shown in FIGS. 12A and 12B.

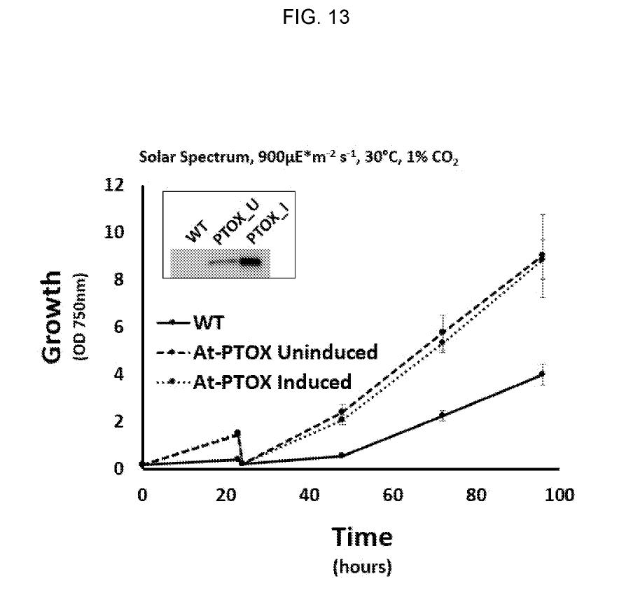

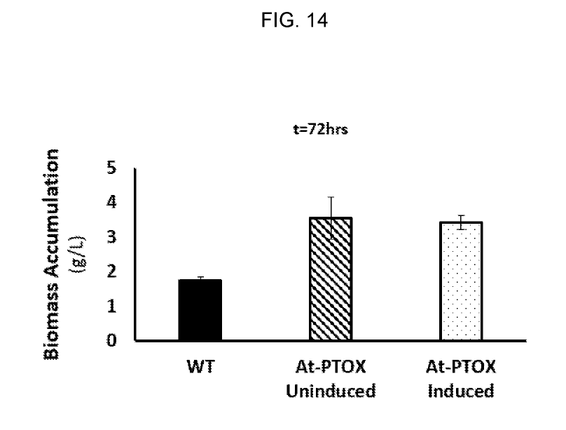

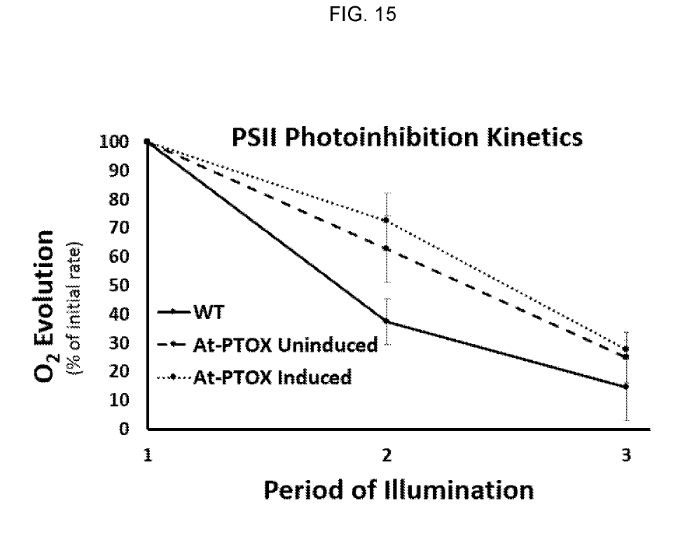

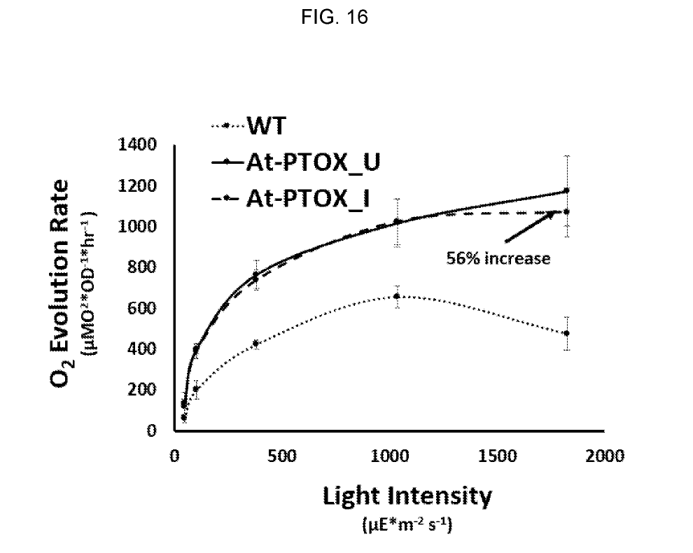

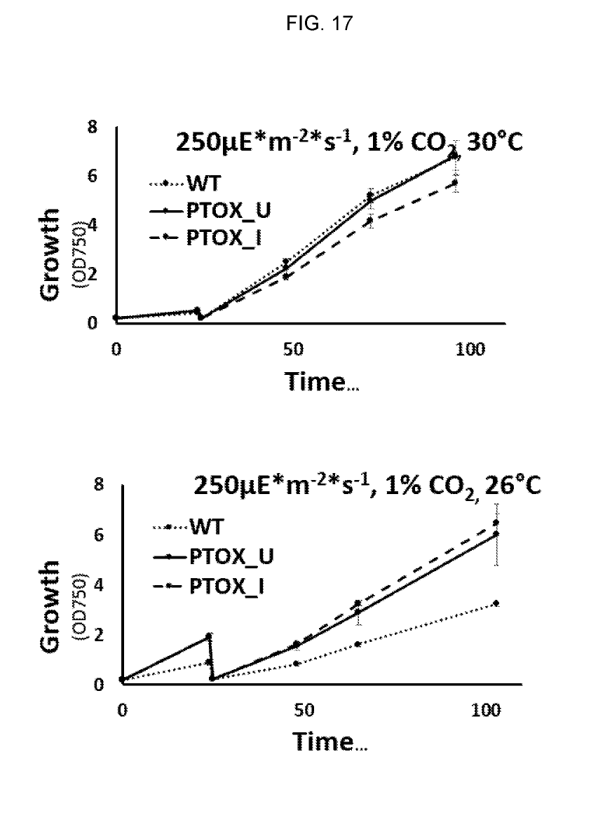

FIG. 13 depicts initial growth experiments under 900 μE*m⁻²*s⁻¹ continuous illumination flux and 1% CO₂/air atmosphere showed improvements in growth rates and yields of the At-PTOX overexpressing strain (MX2394) relative to respective WT control. FIG. 14 depicts dry weights taken 72 hours after inoculation. FIG. 15 depicts alterations in oxygen evolution after the application of 5 minute periods of illumination (x-axis). FIG. 16 depicts rate of oxygen evolution of MX2349 (At-PTOX) and WT shown at increasing light intensities. FIG. 17 depicts initial growth experiments under 250 μE*m⁻²*s⁻¹ continuous illumination flux and 1% CO₂/air atmosphere showed improvements in growth rates and yields of the At-PTOX overexpressing strain (MX2349) relative to respective WT control at 26° C. (a non-optimum temperature for this strain).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically-significant reduction in a modified Cyanobacteria's increased growth and biomass yield in high light conditions using research protocols and measures disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, if references have been made to patents, printed publications, journal articles and other written text throughout this specification (referenced materials herein), each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A photosynthetic microorganism comprising a genetic modification wherein the genetic modification comprises insertion of an exogenous nucleotide sequence that (i) upregulates PTOX; (ii) upregulates Flv3; and/or (iii) upregulates PTOX and Flv3 wherein the genetic modification results in decreased photodamage and/or increased growth, and/or biomass yield in high light conditions as compared to a photosynthetic microorganism of the same species without the genetic modification in high light conditions.

2. A photosynthetic microorganism of claim 1 comprising upregulated PTOX resulting from over-expression of at least one endogenous nucleotide sequence encoding the PTOX and/or at least one exogenous nucleotide sequence encoding the PTOX.

3. A photosynthetic microorganism of claim 2 wherein the up-regulated PTOX comprises SEQ ID NO: 47.

4. A modified photosynthetic microorganism of claim 2 wherein the exogenous nucleotide sequence comprises SEQ ID NO: 46.

5. A photosynthetic microorganism of claim 1 comprising upregulated Flv3 resulting from over-expression of at least one endogenous nucleotide sequence encoding the Flv3 and/or at least one exogenous nucleotide sequence encoding the Flv3.

6. A photosynthetic microorganism of claim 5 wherein the up-regulated Flv3 comprises SEQ ID NO: 39 and/or SEQ ID NO: 40.

7. A photosynthetic microorganism of claim 5 wherein the exogenous nucleotide sequence comprises SEQ ID NO: 52 and/or SEQ ID NO: 53.

8. A photosynthetic microorganism of claim 1 wherein the photosynthetic microorganism shows decreased photodamage in high light conditions.

9. A photosynthetic microorganism of claim 1 wherein the photosynthetic microorganism shows increased ATP production in high light conditions.

10. A photosynthetic microorganism of claim 1 wherein the photosynthetic microorganism shows increased growth in high light conditions and/or low temperature growth conditions.

11. A photosynthetic microorganism of claim 1 wherein the photosynthetic microorganism shows increased biomass yield in high light conditions and/or low temperature growth conditions.

12. A photosynthetic microorganism of claim 1 having decreased RpaB pathway activity.

13. A photosynthetic microorganism of claim 12 wherein the decreased RpaB pathway activity results from expression of N-RpaB.

14. A modified photosynthetic microorganism comprising a modification which results in decreased photodamage and/or increased growth, and/or biomass yield in high light conditions as compared to a photosynthetic microorganism of the same species without the modification in high light conditions.

15. A modified photosynthetic microorganism of claim 14 wherein the modified photosynthetic microorganism is a genetically-modified photosynthetic microorganism.

16. A modified photosynthetic microorganism of claim 14 modified to overexpress an endogenous NADPH escape valve gene.

17. A modified photosynthetic microorganism of claim 14 comprising at least one exogenous nucleotide sequence.

18. A modified photosynthetic microorganism of claim 17 wherein the exogenous nucleotide sequence upregulates expression of an endogenous NADPH escape valve gene.

19. A modified photosynthetic microorganism of claim 14 wherein the decreased photodamage and/or increased growth, and/or biomass yield in high light conditions results from up-regulation of at least one endogenous nucleotide sequence encoding an NADPH escape valve and/or at least one exogenous nucleotide sequence encoding an NADPH escape valve.

20. A modified photosynthetic microorganism of claim 20 wherein the at least one NADPH escape valve comprises (i) Flv3, (ii) Flv3 in combination with Flv1, (iii) a hox hydrogenase, (iv) a transhydrogenase, and/or (v) PTOX.

21. A modified photosynthetic microorganism of claim 20 wherein the up-regulated NADPH escape valve comprises upregulated transhydrogenase resulting from over-expression of at least one endogenous nucleotide sequence encoding the transhydrogenase and/or at least one exogenous nucleotide sequence encoding the transhydrogenase.

22. A modified photosynthetic microorganism of claim 21 wherein the up-regulated transhydrogenase results from over-expression of at least one exogenous nucleotide sequence encoding transhydrogenase subunits.

23. A modified photosynthetic microorganism of claim 21 wherein the up-regulated transhydrogenase comprises SEQ ID NO: 34, SEQ ID NO: 36, and/or SEQ ID NO: 38.

24. A modified photosynthetic microorganism of claim 21 wherein the up-regulated transhydrogenase comprises SEQ ID NO: 34, SEQ ID NO: 36, and SEQ ID NO: 38.

25. A modified photosynthetic microorganism of claim 21 wherein the exogenous nucleotide sequence comprises SEQ ID NO: 33, SEQ ID NO: 35, and/or SEQ ID NO: 37.

26. A modified photosynthetic microorganism of claim 21 wherein the exogenous nucleotide sequence comprises SEQ ID NO: 33, SEQ ID NO: 35, and SEQ ID NO: 37.

27. A modified photosynthetic microorganism of claim 20 wherein the up-regulated NADPH escape valve includes upregulated PTOX resulting from over-expression of at least one endogenous nucleotide sequence encoding the PTOX and/or at least one exogenous nucleotide sequence encoding the PTOX.

28. A modified photosynthetic microorganism of claim 27 wherein the up-regulated PTOX comprises SEQ ID NO: 47.

29. A modified photosynthetic microorganism of claim 27 wherein the exogenous nucleotide sequence comprises SEQ ID NO: 46.

30. A modified photosynthetic microorganism of claim 20 comprising upregulated Flv3 resulting from over-expression of at least one endogenous nucleotide sequence encoding the Flv3 and/or at least one exogenous nucleotide sequence encoding the Flv3.

31. A modified photosynthetic microorganism of claim 30 wherein the up-regulated Flv3 comprises SEQ ID NO: 39 and/or SEQ ID NO: 40.

32. A modified photosynthetic microorganism of claim 30 wherein the exogenous nucleotide sequence comprises SEQ ID NO: 52 and/or SEQ ID NO: 53.

33. A modified photosynthetic microorganism of claim 16 comprising an exogenous nucleotide sequence encoding an NADPH escape valve behind an inducible or constitutive promoter.

34. A modified photosynthetic microorganism of claim 16 comprising an exogenous nucleotide sequence encoding an NADPH escape valve behind an inducible or constitutive promoter wherein the exogenous nucleotide sequence is inserted in neutral site 1 (NS1), neutral site 2 (NS2), neutral site 3 (NS3), neutral site 4 (NS4), or neutral site 5 (NS5).

35. A modified photosynthetic microorganism of claim 16 wherein the photosynthetic microorganism shows decreased photodamage in high light conditions.

36. A modified photosynthetic microorganism of claim 16 wherein the photosynthetic microorganism shows increased ATP production in high light conditions.

37. A modified photosynthetic microorganism of claim 16 wherein the photosynthetic microorganism shows increased growth in high light conditions and/or low temperature growth conditions.

38. A modified photosynthetic microorganism of claim 16 wherein the photosynthetic microorganism shows increased biomass yield in high light conditions and/or low temperature growth conditions.

39. A modified photosynthetic microorganism of 16 having decreased RpaB pathway activity.

40. A modified photosynthetic microorganism of claim 39 wherein the decreased RpaB pathway activity results from (i) expression of at least one exogenous nucleotide sequence and/or (ii) a wild-type nucleotide sequence deletion wherein (i) and/or (ii) decreases RpaB pathway activity as compared to a photosynthetic microorganism of the same species without the modification(s).

41. A modified photosynthetic microorganism of claim 40 wherein one or more exogenous nucleotide sequences (i) result in translation of an incomplete or unstable RpaB protein; (ii) result in translation of an RpaB protein that folds incorrectly; (iii) reduce transcription of the RpaB gene; (iv) result in incomplete transcription of the RpaB gene; (v) interfere with an encoded RpaB RNA transcript and/or (vi) reduce translation of RpaB.

42. A modified photosynthetic microorganism of claim 40 wherein one or more exogenous nucleotide sequences include a foreign set of base pairs inserted or substituted into the RpaB coding region.

43. A modified photosynthetic microorganism of claim 40 wherein one or more exogenous nucleotide sequences include an antisense sequence that interferes with transcription or translation of the RpaB gene.

44. A modified photosynthetic microorganism of claim 40 wherein one or more exogenous nucleotide sequences expresses an RpaB decoy under the control of a promoter.

45. A modified photosynthetic microorganism of claim 44 wherein the RpaB decoy is a fragment of wild type RpaB comprising Asp56 or a conservative substitution thereof.

46. A modified photosynthetic microorganism of claim 44 wherein the RpaB decoy is N-RpaB.

47. A modified photosynthetic microorganism of claim 44 wherein the RpaB decoy is an N-RpaB variant that maintains Asp56 or a conservative substitution thereof.

48. A modified photosynthetic microorganism of claim 16 wherein the modified photosynthetic microorganism is a Cyanobacteria.

49. A modified photosynthetic microorganism of claim 16 wherein the modified photosynthetic microorganism is a Cyanobacteria selected from Synechococcus elongatus, Arthrospira maxima, Arthrospira platensis, and Cyanobacterium aponinum.

50. A method for decreasing photodamage and/or increasing growth and/or biomass yield of a photosynthetic microorganism in high light conditions comprising modifying the photosynthetic microorganism to up-regulate at least one NADPH escape valve as compared to a photosynthetic microorganism of the same species without the modification in high light conditions.

51. A method of claim 50 wherein the modifying comprises genetically modifying.

52. A method of claim 50 wherein the modifying comprises initiating over-expression of at least one endogenous NADPH escape valve gene and/or at least one exogenous NADPH escape valve gene.

53. A method of claim 50 wherein the modifying comprises inserting at least one exogenous nucleotide sequence into the photosynthetic microorganism.

54. A method of claim 50 wherein the at least one NADPH escape valve comprises (i) Flv3, (ii) Flv3 in combination with Flv1, (iii) a hox hydrogenase, (iv) a transhydrogenase, and/or (v) PTOX.

55. A method of claim 54 wherein the at least one up-regulated NADPH escape valve comprises a transhydrogenase.

56. A method of claim 55 wherein the up-regulated transhydrogenase results from over-expressing at least one endogenous nucleotide sequence encoding the transhydrogenase and/or at least one exogenous nucleotide sequence encoding the transhydrogenase.

57. A method of claim 56 wherein the up-regulated transhydrogenase results from over-expressing at least one exogenous nucleotide sequence encoding transhydrogenase subunits.

58. A method of claim 55 wherein the up-regulated transhydrogenase comprises SEQ ID NO: 34, SEQ ID NO: 36, and/or SEQ ID NO: 38.

59. A method of claim 55 wherein the up-regulated transhydrogenase comprises SEQ ID NO: 34, SEQ ID NO: 36, and SEQ ID NO: 38.

60. A method of claim 55 wherein the exogenous nucleotide sequence comprises SEQ ID NO: 33, SEQ ID NO: 35, and/or SEQ ID NO: 37.

61. A method of claim 55 wherein the exogenous nucleotide sequence comprises SEQ ID NO: 33, SEQ ID NO: 35, and SEQ ID NO: 37.

62. A method of claim 54 wherein the up-regulated NADPH escape valve comprises upregulated PTOX resulting from over-expression of at least one endogenous nucleotide sequence encoding the PTOX and/or at least one exogenous nucleotide sequence encoding the PTOX.

63. A method of claim 62 wherein the up-regulated PTOX comprises SEQ ID NO 47:

64. A method of claim 62 wherein the exogenous nucleotide sequence comprises SEQ ID NO: 46.

65. A method of claim 54 wherein the up-regulated NADPH escape valve comprises upregulated Flv3 resulting from over-expression of at least one endogenous nucleotide sequence encoding the Flv3 and/or at least one exogenous nucleotide sequence encoding the Flv3.

66. A method of claim 65 wherein the up-regulated Flv3 comprises SEQ ID NO: 39 and/or SEQ ID NO: 40.

67. A method of claim 65 wherein the exogenous nucleotide sequence comprises SEQ ID NO: 52 and/or SEQ ID NO: 53.

68. A method of claim 50 wherein the modified photosynthetic microorganism shows reduced photodamage in high light conditions.

69. A method of claim 50 wherein the photosynthetic microorganism shows increased ATP production in high light conditions.

70. A method of claim 50 wherein the photosynthetic microorganism shows increased growth in high light conditions and/or low temperature growth conditions.

71. A method of claim 50 wherein the photosynthetic microorganism shows increased biomass yield in high light conditions and/or low temperature growth conditions.

72. A method of claim 50 further comprising modifying the photosynthetic microorganism to down-regulate RpaB pathway activity within the photosynthetic microorganism as compared to a photosynthetic microorganism of the same species without the modification.

73. A method of claim 72 wherein the RpaB pathway modifying comprises genetically modifying the photosynthetic microorganism.

74. A method of claim 73 wherein the RpaB pathway modifying comprises inserting at least one exogenous nucleotide sequence into the photosynthetic microorganism or deleting an endogenous nucleotide sequence from the photosynthetic microorganism.

75. A method of claim 74 wherein the at least one exogenous nucleotide sequence (i) results in translation of an incomplete or unstable RpaB protein; (ii) results in translation of an RpaB protein that folds incorrectly; (iii) reduces transcription of the RpaB gene; (iv) results in incomplete transcription of the RpaB gene; (v) interferes with an encoded RpaB RNA transcript and/or (vi) reduces translation of RpaB.

76. A method of claim 72 wherein the RpaB pathway modifying comprises inserting or substituting a foreign set of base pairs into the RpaB coding region.

77. A method of any claim 72 wherein the RpaB pathway modifying comprises inserting an antisense sequence that interferes with transcription or translation of the RpaB gene.

78. A method of claim 72 wherein the RpaB pathway modifying comprises inserting an exogenous nucleotide sequence that expresses an RpaB decoy under the control of a promoter.

79. A method of claim 72 wherein the RpaB pathway modifying comprises inserting an exogenous nucleotide sequence that expresses an N-terminal fragment of wild type RpaB comprising Asp56 or a conservative substitution thereof.

80. A method of claim 72 wherein the RpaB pathway modifying comprises inserting an exogenous nucleotide sequence that expresses N-RpaB.

81. A method of any claim 72 wherein the RpaB pathway modifying comprises inserting an exogenous nucleotide sequence that expresses an N-RpaB variant that maintains Asp56 or a conservative substitution thereof.

82. A method of claim 72 wherein the modifying and/or RpaB pathway modifying comprises modifying a Cyanobacteria.

83. A method of claim 72 wherein the modifying and/or RpaB pathway modifying comprises modifying a Cyanobacteria selected from Synechococcus elongatus, Arthrospira maxima, Arthrospira platensis, and Cyanobacterium aponinum.
